(19) United States
(12) Patent Application Publication
(10) Pub. No.: US 2010/0313310 A1
(43) Pub. Date: Dec. 9, 2010

(54) METHOD FOR IMPROVED UTILIZATION OF THE PRODUCTION POTENTIAL OF TRANSGENIC PLANTS

(75) Inventors: Wolfram Andersch, Bergisch Gladbach (DE); Reiner Fischer, Monheim (DE); Anton Kraus, Leichlingen (DE); Heike Hungenberg, Langenfeld (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer CropScience AG, Monheim (DE)

(21) Appl. No.: 12/520,714

(22) PCT Filed: Dec. 15, 2007
(86) PCT No.: PCT/EP2007/011037

§ 371 Date: Mar. 29, 2010

(30) Foreign Application Priority Data
Dec. 27, 2006 (DE) 10 2006 061 621.9
Mar. 1, 2007 (DE) 10 2007 009 957.8

(51) Int. Cl. A01H 5/00; A01N 43/38; A01P 7/04

(52) U.S. Cl. 800/298; 514/409; 800/295

(57) ABSTRACT

The invention relates to a method for improving the utilization of the production potential of transgenic plants.

In the last years, there has been a marked increase in the proportion of transgenic plants in agriculture, even if regional differences are still noticeable to date. Thus, for example, the proportion of transgenic maize in the USA has doubled from 26% to 52% since 2001, while transgenic maize has hardly been of any practical importance in Germany. However, in other European countries, for example in Spain, the proportion of transgenic maize is already about 12%.

The invention relates to a method for improving the utilization of the production potential of transgenic plants.

In the last years, there has been a marked increase in the proportion of transgenic plants in agriculture, even if regional differences are still noticeable to date. Thus, for example, the proportion of transgenic maize in the USA has doubled from 26% to 52% since 2001, while transgenic maize has hardly been of any practical importance in Germany. However, in other European countries, for example in Spain, the proportion of transgenic maize is already about 12%.

Transgenic plants are employed mainly to utilize the production potential of respective plant varieties in the most favourable manner, at the lowest possible input of production means. The aim of the genetic modification of the plants is in particular the generation of resistance in the plants to certain pests or harmful organisms or else herbicides and also to abiotic stress (for example drought, heat or elevated salt levels). It is also possible to modify a plant genetically to increase certain quality or product features, such as, for example, the content of selected vitamins or oils, or to improve certain fibre properties.

Herbicide resistance or tolerance can be achieved, for example, by incorporating genes into the useful plant for expressing enzymes to detoxify certain herbicides, so that a relatively unimpeded growth of these plants is possible even in the presence of these herbicides for controlling broad-leaved weeds and weed grasses. Examples which may be mentioned are cotton varieties or maize varieties which tolerate the herbicidally active compound glyphosate (Roundup®), (Roundup Ready®, Monsanto) or the herbicides glufosinate or oxynil.

More recently, there has also been the development of useful plants comprising two or more genetic modifications (“stacked transgenic plants” or multiply transgenic crops). Thus, for example, Monsanto has developed multiply transgenic maize varieties which are resistant to the European corn borer (Ostrinia nubilalis) and the Western corn rootworm (Diabrotica virgifera). Also known are maize and cotton crops which are both resistant to the Western corn rootworm and the cotton bollworm and tolerant to the herbicide Roundup®.

It has now been found that the utilization of the production potential of transgenic useful plants can be improved even more by treating the plants with one or more 3-arylpyrrolidine-2,4-dione derivative(s). Here, the term “treatment” includes all measures resulting in a contact between these active compounds and at least one plant part. “Plant parts” are to be understood as meaning all above-ground and below-ground parts and organs of plants, such as shoot, leaf, flower and root, by way of example leaves, needles, stalks, stems, flowers, fruit bodies, fruits and seed, and also roots, tubers and rhizomes. The plant parts also include harvested material and also vegetative and generative propagation material, for example cuttings, tubers, rhizomes, slips and seed.

3-Arylpyrrolidine-2,4-dione derivatives and their herbicidal or insecticidal actions are extensively known from the prior art. Thus, for example, EP-A-355 599 and EP-A-415 211 disclose bicyclic 3-arylpyrrolidine-2,4-dione derivatives. Substituted monocyclic 3-arylpyrrolidine-2,4-dione derivatives are known from EP-A-377 893 and EP-A-442 077. Furthermore known are polycyclic 3-arylpyrrolidine-2,4-dione derivatives (EP-A-442 073) and also tetramic acid derivatives from EP-A-456 063, EP-A-521 334, EP-A-596 298, EP-A-613 884, WO 95/01 997, WO 95/26 954, WO 95/20 572, EP-A-0 668 267, WO 96/25 395, WO 96/35 664, WO 97/01 535, WO 97/02 243, WO 97/36 868, WO 97/43 275, WO 98/05 638, WO 98/06 721, WO 98/25 928, WO 99/16 748, WO 99/24 437, WO 99/43 649, WO 99/48 869, WO 99/55 673, WO 01/09 092, WO 91/17 972, WO 01/23 354, WO 01/74 770, WO 03/013 249, WO 2004/007 448, WO 2004/024 688, WO 04/065 366, WO 04/080 962, WO 04/111 042, WO 05/044 791, WO 05/044 796, WO 05/048 710, WO 05/049 596, WO 05/066 125, WO 05/092 897, WO 06/000 355, WO 06/029799, WO 06/056281 and WO 06/056282.

From these documents, the person skilled in the art will easily be familiar with processes for producing and methods for applying 3-arylpyrrolidine-2,4-dione derivatives (3-APD), and with their action. Accordingly, these documents are incorporated into the present application in their entirety with respect to the active compounds which can be employed according to the invention, and to their preparation and use.

The 3-APD which can be employed according to the invention have the general formula (I), as follows:

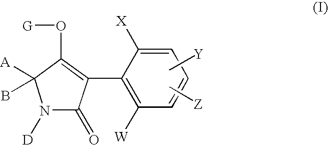

in which
X represents halogen, alkyl, alkoxy, haloalkyl, haloalkoxy or cyano,
W, Y and Z independently of one another represent hydrogen, halogen, alkyl, alkoxy, haloalkyl, haloalkoxy or cyano,
A represents hydrogen, in each case optionally halogen-substituted alkyl, alkoxyalkyl, saturated, optionally substituted cycloalkyl in which optionally at least one ring atom is replaced by a heteroatom,
B represents hydrogen or alkyl,
A and B together with the carbon atom to which they are attached represent a saturated or unsaturated substituted or unsubstituted cycle which optionally contains at least one heteroatom,
D represents hydrogen or an optionally substituted radical from the group consisting of alkyl, alkenyl, alkoxyalkyl, saturated cycloalkyl in which optionally one or more ring members are replaced by heteroatoms,
A and D together with the atoms to which they are attached represent a saturated or unsaturated cycle which is unsubstituted or substituted in the A,D moiety and optionally contains at least one heteroatom,
G represents hydrogen (a) or represents one of the groups

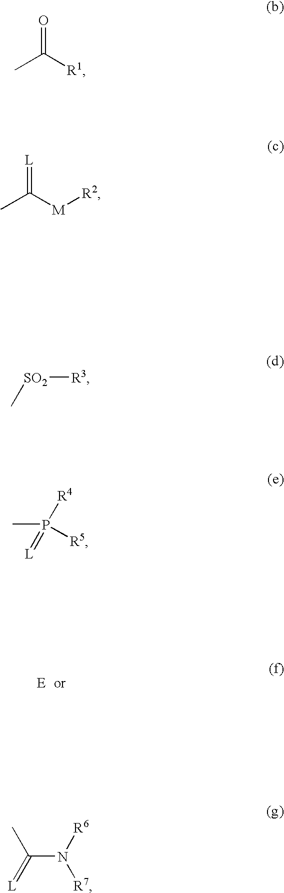

in which
E represents a metal ion or an ammonium ion,
L represents oxygen or sulphur,
M represents oxygen or sulphur,
R¹ represents in each case optionally halogen-substituted alkyl, alkenyl, alkoxyalkyl, alkylhioalkyl, polyalkoxyalkyl or optionally halogen-, alkyl- or alkoxy-substituted cycloalkyl which may be interrupted by at least one heteroatom, represents in each case optionally substituted phenyl, phenylalkyl, hetaryl, phenoxyalkyl or hetaryloxyalkyl,
R² represents in each case optionally halogen-substituted alkyl, alkenyl, alkoxyalkyl, polyalkoxyalkyl or represents in each case optionally substituted cycloalkyl, phenyl or benzyl,
R³ represents optionally halogen-substituted alkyl or optionally substituted phenyl,
R⁴ and R⁵ independently of one another represent in each case optionally halogen-substituted alkyl, alkoxy, alkylamino, dialkylamino, alkylthio, alkenylthio, cycloalkylthio or represent in each case optionally substituted phenyl, benzyl, phenoxy or phenylthio and
R⁶ and R⁷ independently of one another represent hydrogen, represent in each case optionally halogen-substituted alkyl, cycloalkyl, alkenyl, alkoxy, alkoxyalkyl, represent optionally substituted phenyl, represent optionally substituted benzyl or together with the nitrogen atom to which they are attached represent an optionally substituted ring which is optionally interrupted by oxygen or sulphur.

In a preferred embodiment of the invention, at least one insecticidally active 3-APD derivative is used for treating transgenic useful plants. For the purpose of the invention, the term “insecticidally active” or “insecticidal” comprises insecticidal, acaricidal, molluscicidal, nematicidal and ovicidal actions, and also a repelling, behaviour-modifying or sterilizing action on pests.

Preferred insecticidally active compounds are compounds of the formula (I), in which
W preferably represents hydrogen, C₁-C₄-alkyl, C₁-C₄-alkoxy, chlorine, bromine or fluorine,
X preferably represents C₁-C₄-alkyl, C₁-C₄-alkoxy, C₁-C₄-haloalkyl, fluorine, chlorine or bromine,
Y and Z independently of one another preferably represent hydrogen, C₁-C₄-alkyl, halogen, C₁-C₄-alkoxy or C₁-C₄-haloalkyl,
A preferably represents hydrogen or in each case optionally halogen-substituted C₁-C₆-alkyl or C₃-C₈-cycloalkyl,
B preferably represents hydrogen, methyl or ethyl,
A, B and the carbon atom to which they are attached preferably represent saturated C₃-C₆-cycloalkyl in which optionally one ring member is replaced by oxygen or sulphur and which is optionally mono- or disubstituted by C₁-C₄-alkyl, trifluoromethyl or C₁-C₄-alkoxy,
D preferably represents hydrogen, in each case optionally fluorine- or chlorine-substituted C₁-Cₒ-alkyl, C₃-C₄-alkenyl or C₃-C₆-cycloalkyl,
A and D together preferably represent in each case optionally methyl-substituted C₃-C₄-alkanediyl in which optionally one methylene group is replaced by sulphur,
G preferably represents hydrogen (a) or represents one of the groups

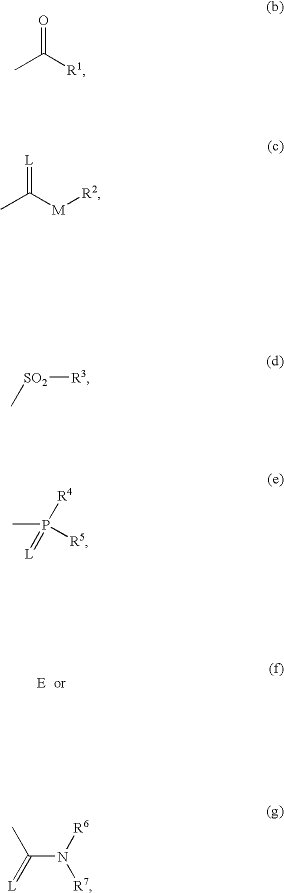

in particular (a), (b), (c) or (g),
in which
E represents a metal ion or an ammonium ion,
L represents oxygen or sulphur and
M represents oxygen or sulphur
R¹ preferably represents in each case optionally halogen-substituted C₁-C₁₀-alkyl, C₂-C₁₀-alkenyl, C₁-C₄-alkoxy-C₁-C₄-alkyl, C₁-C₄-alkylthio-C₁-C₄-alkyl or optionally fluorine-, chlorine-, C₁-C₁-alkyl- or C₁-C₂-alkoxy-substituted C₃-C₆-cycloalkyl, represents optionally fluorine-, chlorine-, bromine-, cyano-, nitro-, C₁-C₄alkyl-, C₁-C₄-alkoxy-, trifluoromethyl- or trifluoromethoxy-substituted phenyl, represents in each case optionally chlorine- or methyl-substituted pyridyl or thienyl,
R² preferably represents in each case optionally fluorine- or chlorine-substituted C₁-C₁₀-alkyl, C₂-C₁₀-alkenyl, C₁-C₄alkoxy-C₂-C₄-alkyl, represents optionally methyl- or methoxy-substituted C₅-C₆-cycloalkyl or represents in each case optionally fluorine-, chlorine-, bromine-, cyano-, nitro-, C₁-C₄-alkyl, C₁-C₄-alkoxy, trifluoromethyl- or trifluoromethoxy-substituted phenyl or benzyl,
R³ preferably represents optionally fluorine-substituted C₁-C₄-alkyl or represents in each case optionally fluorine-, chlorine-, bromine-, C₁-C₄-alkyl, C₁-C₄-alkoxy, trifluoromethyl-, trifluoromethoxy-, cyano- or nitro-substituted phenyl,
R⁴ preferably represents in each case optionally fluorine- or chlorine-substituted C₁-C₄-alkyl, C₁-C₄-alkoxy, C₁-C₄-alkylamino, C₁-C₄-alkylthio or represents in each case optionally fluorine-, chlorine-, bromine-, nitro-, cyano-, C₁-C₄alkoxy-, trifluoromethoxy-, C₁-C₄-alkylthio-, C₁-C₄-haloalkylthio-, C₁-C₄-alkyl- or trifluoromethyl-substituted phenyl, phenoxy or phenylthio,
R⁵ preferably represents C₁-C₄-alkoxy or C₁-C₄-thioalkyl,

R⁶ preferably represents C₁-C₆-alkyl, C₃-C₆-cycloalkyl, C₁-C₆-alkoxy, C₃-C₆-alkenyl, C₁-C₄-alkoxy-C₁-C₄alkyl,
R⁷ preferably represents C₁-C₆-alkyl, C₃-C₆-alkenyl or C₁-C₄-alkoxy-C₁-C₄-alkyl,
R⁶ and R⁷ together preferably represent an optionally methyl- or ethyl-substituted C₃-C₆-alkylene radical in which optionally one carbon atom is replaced by oxygen or sulphur.
   Particular preference is given to compounds of the formula (I), in which
W particularly preferably represents hydrogen, methyl, ethyl, chlorine, bromine or methoxy,
X particularly preferably represents chlorine, bromine, methyl, ethyl, propyl, i-propyl, methoxy, ethoxy or trifluoromethyl,
Y and Z particularly preferably independently of one another represent hydrogen, fluorine, chlorine, bromine, methyl, ethyl, propyl, i-propyl, trifluoromethyl or methoxy,
A particularly preferably represents methyl, ethyl, propyl, i-propyl, butyl, i-butyl, sec-butyl, tert-butyl, cyclopropyl, cyclopentyl or cyclohexyl,
B particularly preferably represents hydrogen, methyl or ethyl,
A, B and the carbon atom to which they are attached particularly preferably represent saturated C₆-cycloalkyl in which optionally one ring member is replaced by oxygen and which is optionally monosubstituted by methyl, ethyl, methoxy, ethoxy, propoxy or butoxy,
D particularly preferably represents hydrogen, represents methyl, ethyl, propyl, i-propyl, butyl, i-butyl, allyl, cyclopropyl, cyclopentyl or cyclohexyl,
A and D together particularly preferably represent optionally methyl-substituted C₃-C₄-alkanediyl,
G particularly preferably represents hydrogen (a) or represents one of the groups

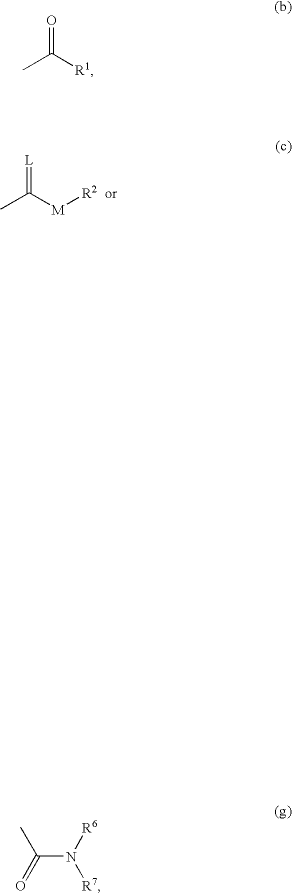

in which
M represents oxygen or sulphur,
R¹ particularly preferably represents C₁-C₈-alkyl, C₂-C₄-alkenyl, methoxymethyl, ethoxymethyl, ethylthiomethyl, cyclopropyl, cyclopentyl or cyclohexyl,
   represents optionally fluorine-, chlorine-, bromine, cyano-, nitro-, methyl-, ethyl-, methoxy-, trifluoromethyl- or trifluoromethoxy-substituted phenyl,
   represents in each case optionally chlorine- or methyl-substituted pyridyl or thienyl,
R² particularly preferably represents C₁-C₈-alkyl, C₂-C₄-alkenyl, methoxyethyl, ethoxyethyl or represents phenyl or benzyl,
R⁶ and R⁷ independently of one another particularly preferably represent methyl, ethyl or together represent a C₅-alkylene radical in which the C₃-methylene group is replaced by oxygen.
   Especially preferred are compounds of the formula (I), in which
W very particularly preferably represents hydrogen or methyl,
X very particularly preferably represents chlorine, bromine or methyl,
Y and Z very particularly preferably independently of one another represent hydrogen, chlorine, bromine or methyl,
A, B and the carbon atom to which they are attached very particularly preferably represent saturated C₆-cycloalkyl in which optionally one ring member is replaced by oxygen and which is optionally monosubstituted by methyl, trifluoromethyl, methoxy, ethoxy, propoxy or butoxy,
G very particularly preferably represents hydrogen (a) or represents one of the groups

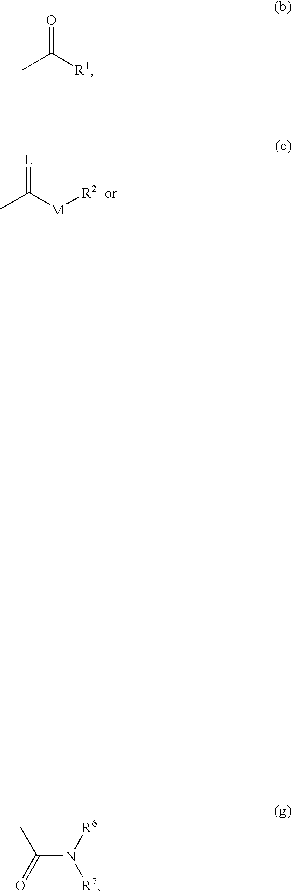

in which
M represents oxygen or sulphur,
R¹ very particularly preferably represents C₁-C₈-alkyl, C₂-C₄-alkenyl, methoxymethyl, ethoxymethyl, ethylthiomethyl, cyclopropyl, cyclopentyl, cyclohexyl or
   represents phenyl which is optionally monosubstituted by fluorine, chlorine, bromine, methyl, methoxy, trifluoromethyl, trifluoromethoxy, cyano or nitro,
   represents in each case optionally chlorine- or methyl-substituted pyridyl or thienyl,
R² very particularly preferably represents C₁-C₈-alkyl, C₂-C₄-alkenyl, methoxyethyl, ethoxyethyl, phenyl or benzyl,
R⁶ and R⁷ independently of one another very particularly preferably represent methyl, ethyl or together represent a C₅-alkylene radical in which the C₃-methylene group is replaced by oxygen.

Depending on the nature of the substitution, the compounds of the formula (I) may also be present as optical isomers or isomer mixtures of varying compositions.

Especially preferred are compounds of the abovementioned formula (I) in which the radicals are as defined below:

(I)

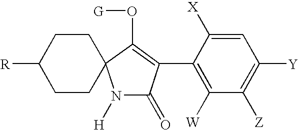

| Exam- | | | | | | | |
|---|---|---|---|---|---|---|---|
| ple | | | | | | | |
| No. | W | X | Y | Z | R | G | m.p. ° C. |
| I-1 | H | Br | H | CH₃ | OCH₃ | CO-i-C₃H₇ | 122 |
| I-2 | H | Br | H | CH₃ | OCH₃ | CO₂—C₂H₅ | 140-142 |
| I-3 | H | CH₃ | H | CH₃ | OCH₃ | H | >220 |
| I-4 | H | CH₃ | H | CH₃ | OCH₃ | CO₂—C₂H₅ | 128 |
| I-5 | CH₃ | CH₃ | H | Br | OCH₃ | H | >220 |
| I-6 | CH₃ | CH₃ | H | Cl | OCH₃ | H | 219 |
| I-7 | H | Br | CH₃ | CH₃ | OCH₃ | CO-i-C₃H₇ | 217 |
| I-8 | H | CH₃ | Cl | CH₃ | OCH₃ | CO₂C₂H₅ | 162 |
| I-9 | CH₃ | CH₃ | CH₃ | CH₃ | OCH₃ | H | >220 |
| I-10 | CH₃ | CH₃ | H | Br | OC₂H₅ | CO-i-C₃H₇ | 212-214 |
| I-11 | H | CH₃ | CH₃ | CH₃ | OC₂H₅ | CO—n-C₃H₇ | 134 |
| I-12 | H | CH₃ | CH₃ | CH₃ | OC₂H₅ | CO-i-C₃H₇ | 108 |
| I-13 | H | CH₃ | CH₃ | CH₃ | OC₂H₅ | CO—c-C₃H₅ | 163 |

in the form of their cis/trans isomer mixtures or their pure cis isomers.

Emphasis is given to the cis isomers of the formulae (I-3) and (I-4)

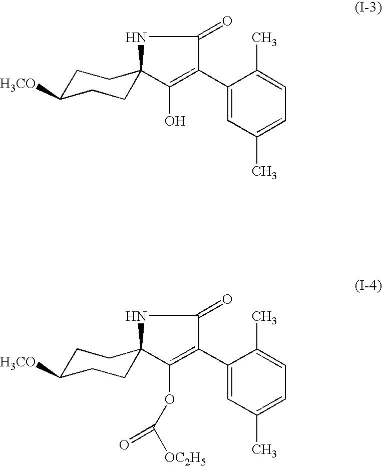

The compounds of the formula (I) are—as already mentioned above—known to the person skilled in the art, as is their preparation (see in particular WO 97/01 535, WO 97/36 868, WO 98/05 638, WO 04/007 448).

Preference is given to mixtures of two or more, preferably two or three, particularly preferably two, of the insecticidally active compounds.

According to the process proposed according to the invention, transgenic plants, in particular the useful plants, are treated with 3-APD derivatives to increase agricultural productivity. For the purpose of the invention, transgenic plants are plants coding for at least one gene or gene fragment not transferred by fertilization. This gene or gene fragment may originate or be derived from another plant of the same species, from plants of a different species, but also from organisms from the animal kingdom or microorganisms (including viruses) (“foreign gene”) and/or, if appropriate, already have mutations compared to the natural sequence. According to the invention, it is also possible to use synthetic genes, which is also included in the term “foreign gene” here. It is also possible for a transgenic plant to code for two or more foreign genes of different origin.

For the purpose of the invention, the “foreign gene” is further characterized in that it comprises a nucleic acid sequence which has a certain biological or chemical function or activity in the transgenic plant. In general, these genes code for biocatalysts, such as, for example, enzymes or ribozymes, or else they comprise regulatory sequences, such as, for example, promoters or terminators, for controlling the expression of endogenous proteins. However, to this end, they may also code for regulatory proteins, such as, for example, repressors or inductors. Furthermore, the foreign gene may also serve the targeted localization of a gene product of the transgenic plant, coding, for example, for a signal sequence. The foreign gene may also code for inhibitors, such as, for example, antisense RNA.

The person skilled in the art is readily familiar with numerous different methods for producing transgenic plants and methods for the targeted mutagenesis, for gene transformation and cloning, for example from: Willmitzer, 1993, Transgenic plants, in: Biotechnology, A Multivolume Comprehensive Treatise, Rehm et al. (eds.), Vol. 2, 627-659, VCH Weinheim, Germany; McCormick et al., 1986, Plant Cell Reports 5: 81-84; EP-A 0221044; EP-A 0131624, or Sambrook et al., 1989, “Molecular Cloning: A Laboratory Manual”, 3rd Ed., Cold Spring Harbor Laboratory Press, Cold Spring Harbor, N.Y.; Winnacker, 1996, “Gene and Klone” [Genes and Clones], 2nd Ed., VCH Weinheim or Christou, 1996, Trends in Plant Science 1: 423-431. Examples of transit or signal peptides or time- or site-specific promoters are disclosed, for example, in Braun et al., 1992, EMBO J. 11: 3219-3227; Wolter et al., 1988, Proc. Natl. Acad. Sci. USA 85: 846-850; Sonnewald et al., 1991, Plant J. 1: 95-106.

An example of a complex genetic manipulation of a useful plant is the so-called GURT technology (“Genetic Use Restriction Technologies”) which allows the technical control of the propagation of the transgenic plant variety in question. To this end, in general two or three foreign genes are cloned into the useful plant which, in a complex interaction after administration of an external stimulus, trigger a cascade resulting in the death of the embryo which would otherwise develop. To this end, the external stimulus (for example an active compound or another chemical or abiotic stimulus) may interact, for example, with a repressor which then no longer suppresses the expression of a recombinase, so that the recombinase is able to cleave an inhibitor thus allowing expression of a toxin causing the embryo to die. Examples of this type of transgenic plants are disclosed in U.S. Pat. No. 5,723,765 or U.S. Pat. No. 5,808,034.

Accordingly, the person skilled in the art is familiar with processes for generating transgenic plants which, by virtue of the integration of regulatory foreign genes and the overexpression, suppression or inhibition of endogenous genes or gene sequences mediated in this manner, if appropriate, or by virtue of the existence or expression of foreign genes or fragments thereof, have modified properties.

As already discussed above, the method according to the invention allows better utilization of the production potential of transgenic plants. On the one hand, this may, if appropriate, be based on the fact that the application rate of the active compound which can be employed according to the invention can be reduced, for example by lowering the dose employed or else by reducing the number of applications. On the other hand, if appropriate, the yield of the useful plants may be increased quantitatively and/or qualitatively. This is true in particular in the case of a transgenically generated resistance to biotic or abiotic stress. If, for example, insecticidal 3-APD are used, the dosage of the insecticide may in certain cases be limited to a sublethal dose, without this resulting in a significant weakening of the desired effect of the active compound on the pests.

Depending on the plant species or plant varieties, their location and the growth conditions (soils, climate, vegetation period, nutrients), these synergistic actions may vary and may be multifarious. Thus possible are, for example, reduced application rates and/or a widening of the activity spectrum and/or an increase of the activity of the compounds and compositions used according to the invention, better plant growth, increased tolerance to high or low temperatures, increased tolerance to drought or to water or soil salt content, increased flowering, easier harvesting, accelerated maturation, higher harvest yields, higher quality and/or higher nutrient value of the harvested products, increased storability and/or processibility of the harvested products, which exceed the effects normally to be expected.

These advantages are the result of a synergistic action, achieved according to the invention, between the 3-APD which can be employed and the respective principle of action of the genetic modification of the transgenic plant. This reduction of production means as a result of the synergism, with simultaneous yield or quality increase, is associated with considerable economical and ecological advantages.

A list of examples known to the person skilled in the art of transgenic plants, with the respective affected structure in the plant or the protein expressed by the genetic modification in the plant being mentioned, is compiled in Table 1. Here, the structure in question or the principle expressed is in each case grouped with a certain feature in the sense of a tolerance to a certain stress factor. A similar list (Table 3) compiles—in a slightly different arrangement—likewise examples of principles of action, tolerances induced thereby and possible useful plants. Further examples of transgenic plants suitable for the treatment according to the invention are compiled in Tables 4, and 5 and 6.

In an advantageous embodiment, the 3-APD are used for treating transgenic plants comprising at least one gene or gene fragment coding for a Bt toxin. A Bt toxin is a protein originating from or derived from the soil bacterium Bacillus thuringiensis which either belongs to the group of the crystal toxins (Cry) or the cytolytic toxins (Cyt). In the bacterium, they are originally formed as protoxins and only metabolized in alkaline medium—for example in the digestive tract of certain feed insects—to their active form. There, the active toxin then binds to certain hydrocarbon structures at cell surfaces causing pores to be formed which destroy the osmotic potential of the cell, which may effect cell lysis. The result is the death of the insects. Bt toxins are active in particular against certain harmful species from the orders of the Lepidoptera (butterflies), Homoptera, Diptera and Coleoptera (beetles) in all their development stages; i.e. from the egg larva via their juvenile forms to their adult forms.

It has been known for a long time that gene sequences coding for Bt toxins, parts thereof or else peptides or proteins derived from Bt toxins can be cloned with the aid of genetical engineering into agriculturally useful plants to generate transgenic plants having endogenous resistance to pests sensitive to Bt toxins. For the purpose of the invention, the transgenic plants coding for a Bt toxin or proteins derived therefrom are defined as “Bt plants”.

The “first generation” of such Bt plants generally only comprise the genes enabling the formation of a certain toxin, thus only providing resistance to one group of pathogens. An example of a commercially available maize variety comprising the gene for forming the Cry1Ab toxin is “YieldGard®” from Monsanto which is resistant to the European corn borer. In contrast, in the Bt cotton variety (Bollgard®), resistance to other pathogens from the family of the Lepidoptera is generated by introduction by cloning of the genes for forming the Cry1Ac toxin. Other transgenic crop plants, in turn, express genes for forming Bt toxins with activity against pathogens from the order of the Coleoptera. Examples that may be mentioned are the Bt potato variety “NewLeaf®” (Monsanto) capable of forming the Cry3A toxin, which is thus resistant to the Colorado potato beetle, and the transgenic maize variety “YieldGard®” (Monsanto) which is capable of forming the Cry 3Bb1 toxin and is thus protected against various species of the Western corn rootworm.

In a “second generation”, the multiply transgenic plants, already described above, expressing or comprising at least two foreign genes were generated.

Preference according to the invention is given to transgenic plants with Bt toxins from the group of the Cry family (see, for example, Crickmore et al., 1998, Microbiol. Mol. Biol. Rev. 62: 807-812), which are particularly effective against Lepidoptera, Coleoptera and Diptera. Examples of genes coding for the proteins are:

cry1Aa1, cry1Aa2, cry1Aa3, cry1Aa4, cry1Aa5, cry1Aa6, cry1Aa7, cry1Aa8, cry1Aa9, cry1Aa10, cry1Aa11 cry1Ab1, cry1Ab2, cry1Ab3, cry1Ab4, cry1Ab5, cry1Ab6, cry1Ab7, cry1Ab8, cry1Ab9, cry1Ab10, cry1Ab10, cry1Ab12, cry1Ab13, cry1Ab14, cry1Ac1, cry1Ac2, cry1Ac3, cry1Ac4, cry1Ac5, cry1Ac6, cry1Ac7, cry1Ac8, cry1Ac9, cry1Ac10, cry1Ac11, cry1Ac12, cry1Ac13, cry1Ad1, cry1Ad2, cry1Ae1, cry1Af1, cry1Ag1, cry1Ba1, cry1Ba2, cry1Bb1, cry1Bc1, cry1Bd1, cry1Be1, cry1Ca1, cry1Ca2, cry1Ca3, cry1Ca4, cry1Ca5, cry1Ca6, cry1Ca7, cry1Cb1, cry1Cb2, cry1Da1, cry1Da2, cry1Db1, cry1Ea1, cry1Ea2, cry1Ea3, cry1Ea4, cry1Ea5, cry1Ea6, cry1Eb1, cry1Fa1, cry1Fa2, cry1Fb1, cry1Fb2, cry1Fb3, cry1Fb4, cry1Ga1, cry1Ga2, cry1Gb1, cry1Gb2, cry1Ha1, cry1Hb1, cry1Ia1, cry1Ia2, cry1Ia3, cry1Ia4, cry1Ia5, cry1Ia6, cry1Ja1, cry1Jb1, cry1Jc1, cry1Ka1, cry1-like, cry2Aa1, cry2Aa2, cry2Aa3, cry2Aa4, cry2Aa5, cry2Aa6, cry2Aa7, cry2Aa8, cry2Aa9, cry2Ab1, cry2Ab2, cry2Ab3, cry2Ac1, cry2Ac2, cry2Ad1, cry3Aa1, cry3Aa2, cry3Aa3, cry3Aa4, cry3Aa5, cry3Aa6, cry3Aa7, cry3Ba1, cry3Ba2, cry3Bb1, cry3Bb2, cry3Bb3, cry3Ca1, cry4Aa1, cry4Aa2, cry4Ba1, cry4Ba2, cry4Ba3, cry4Ba4, cry5Aa1, cry5Ab1, cry5Ac1, cry5Ba1, cry6Aa1, cry6Ba1, cry7Aa1, cry7Ab1, cry7Ab2, cry8Aa1, cry8Ba1, cry8Ca1, cry9Aa1, cry9Aa2, cry9Ba1, cry9Ca1, cry9Da1, cry9Da2, cry9Ea1, cry9 like, cry10Aa1, cry10Aa2, cry11Aa1, cry11Aa2, cry11Ba1, cry11Bb1, cry12Aa1, cry13Aa1, cry14Aa1, cry15Aa1, cry16Aa1, cry17Aa1, cry18Aa1, cry18Ba1, cry18Ca1, cry19Aa1, cry19Ba1, cry20Aa1, cry21Aa1, cry21Aa2, cry22Aa1, cry23Aa1, cry24Aa1, cry25Aa1, cry26Aa1, cry27Aa1, cry28Aa1, cry28Aa2, cry29Aa1, cry30Aa1, cry31Aa1, cyt1Aa1, cyt1Aa2, cyt1Aa3, cyt1Aa4, cyt1Ab1, cyt1Ba1, cyt2Aa1, cyt2Ba1, cyt2Ba2, cyt2Ba3, cyt2Ba4, cyt2Ba5, cyt2Ba6, cyt2Ba7, cyt2Ba8, cyt2Bb1.

Particular preference is given to the genes or gene sections of the subfamilies cry1, cry2, cry3, cry5 and cry9; especially preferred are cry1Ab, cry1Ac, cry3A, cry3B and cry9C.

Furthermore, it is preferred to use plants which, in addition to the genes for one or more Bt toxins, express or contain, if appropriate, also genes for expressing, for example, a protease or peptidase inhibitor (such as in WO-A 95/35031), of herbicide resistances (for example to glufosinate or glyphosate by expression of the pat gene or bar gene) or for becoming resistant to nematodes, fungi or viruses (for example by expressing a gluconase, chitinase). However, they may also be modified in their metabolic properties, so that they show a qualitative and/or quantitative change of ingredients (for example by modification of the energy, carbohydrate, fatty acid or nitrogen metabolism or by metabolite currents influencing these (see above).

A list of examples of principles of action which can be introduced by genetic modification into a useful plant and which are suitable for the treatment according to the invention on their own or in combination is compiled in Table 2. Under the header “AP” (active principle), this table contains the respective principle of action and associated therewith the pest to be controlled.

In a particularly preferred variant, the process according to the invention is used for treating transgenic vegetable, maize, soyabean, cotton, tobacco, rice, potato and sugar beet varieties. These are preferably Bt plants.

The vegetable plants or varieties are, for example, the following useful plants:
potatoes: preferably starch potatoes, sweet potatoes and table potatoes;
root vegetables: preferably carrots, turnips (swedes, stubble turnips (Brassica rapa var. rapa), spring turnips, autumn turnips (Brassica campestris ssp. rapifera), Brassica rapa L. ssp. rapa f. teltowiensis), scorzonera, Jerusalem artichoke, turnip-rooted parsley, parsnip, radish and horseradish;
tuber vegetables: preferably kohlrabi, beetroot, celeriac, garden radish;
bulb crops: preferably scallion, leek and onions (planting onions and seed onions);
brassica vegetables: preferably headed cabbage (white cabbage, red cabbage, kale, savoy cabbage), cauliflowers, broccoli, curly kale, marrow-stem kale, seakale and Brussels sprouts;
fruiting vegetables: preferably tomatoes (outdoor tomatoes, vine-ripened tomatoes, beef tomatoes, greenhouse tomatoes, cocktail tomatoes, industrial and fresh market tomatoes), melons, eggplants, aubergines, pepper (sweet pepper and hot pepper, Spanish pepper), chilli pepper, pumpkins, courgettes and cucumbers (outdoor cucumbers, greenhouse cucumbers snake gourds and gherkins);
vegetable pulses: preferably bush beans (as sword beans, string beans, flageolet beans, wax beans, corn beans of green- and yellow-podded cultivars), pole beans (as sword beans, string beans, flageolet beans, wax beans of green-, blue- and yellow-podded cultivars), broadbeans (field beans, Windsor beans, cultivars having white- and black-spotted flowers), peas (chickling vetch, chickpeas, marrow peas, shelling peas, sugar-peas, smooth peas, cultivars having light- and dark-green fresh fruits) and lentils;
green vegetables and stem vegetables: preferably Chinese cabbage, round-headed garden lettuce, curled lettuce, lamb's-lettuce, iceberg lettuce, romaine lettuce, oakleaf lettuce, endives, radicchio, lollo rossa, ruccola lettuce, chicory, spinach, chard (leaf chard and stem chard) and parsley;
other vegetables: preferably asparagus, rhubarb, chives, artichokes, mint varieties, sunflowers, Florence fennel, dill, garden cress, mustard, poppy seed, peanuts, sesame and salad chicory.

Bt vegetables including exemplary methods for preparing them are described in detail, for example, in Barton et al., 1987, Plant Physiol. 85: 1103-1109; Vaeck et al., 1987, Nature 328: 33-37; Fischhoff et al., 1987, Bio/Technology 5: 807-813. In addition, Bt vegetable plants are already known as commercial varieties, for example the potato cultivar NewLeaf® (Monsanto). The preparation of Bt vegetables is also described in U.S. Pat. No. 6,072,105.

Likewise, Bt cotton is already cotton in principle, for example from U.S. Pat. No. 5,322,938 or from Prietro-Samsonór et al., J. Ind. Microbiol. & Biotechn. 1997, 19, 202, and H. Agaisse and D. Lereclus, J. Bacteriol. 1996, 177, 6027. Different varieties of Bt cotton, too, are already commercially available, for example under the name NuCOTN® (Deltapine (USA)). In the context of the present invention, particular preference is given to Bt cotton NuCOTN33® and NuCOTN33B®.

The use and preparation of Bt maize has likewise already been known for a long time, for example from Ishida, Y., Saito, H., Ohta, S., Hiei, Y., Komari, T., and Kumashiro, T. (1996). High efficiency transformation of maize (Zea mayz L.) mediated by Agrobacterium tumefaciens. Nature Biotechnology 4: 745-750. EP-B-0485506, too, describes the preparation of Bt maize plants. Furthermore, different varieties of Bt maize are commercially available, for example under the following names (company/companies is/are in each case given in brackets): KnockOut® (Novartis Seeds), NaturGard® (Mycogen Seeds), Yieldgard® (Novartis Seeds, Monsanto, Cargill, Golden Harvest, Pioneer, DeKalb inter alia), Bt-Xtra® (DeKalb) and StarLink® (Aventis CropScience, Garst inter alia). For the purpose of the present invention, particular preference is given especially to the following maize cultivars: KnockOut®, NaturGard®, Yieldgard®, Bt-Xtra® and StarLink®.

For soyabeans, too, Roundup®Ready cultivar or cultivars resistant to the herbicide Liberty Link® are available and can be treated according to the invention. In the case of rice, a large number of “Golden Rice” lines are available which are likewise characterized in that, by virtue of a transgenic modification, they have an increased content of provitamin A. They, too, are examples of plants which can be treated by the method according to the invention, with the advantages described.

The method according to the invention is suitable for controlling a large number of harmful organisms which occur in particular in vegetables, maize and cotton, in particular insects and arachnids, very particularly preferably insects. The pests mentioned include:
from the order of the Isopoda: for example Armadillidium spp., Oniscus spp., Porcellio spp.
from the order of the Diplopoda: for example Blaniulus spp.
from the order of the Chilopoda for example Geophilus spp., Scutigera spp.
from the order of the Symphyla: for example Scutigerella spp.
from the order of the Thysanura: for example Lepisma spp.
from the order of the Collembola: for example Onychiurus spp.
from the order of the Orthoptera: for example Blattella spp., Periplaneta spp., Leucophaea spp., Acheta spp., Gryllotalpa spp., Locusta migratoria migratorioides, Melanoplus spp., Schistocerca spp.
from the order of the Dermaptera: for example Forficula spp.
from the order of the Isoptera: for example Reticulitermes spp., Reticulitermes spp., Coptotermes spp.
from the order of the Thysanoptera: for example Frankliniella spp., Kakothrips spp., Hercinothrips spp., Scirtothrips spp., Taeniothrips spp., Thrips spp.
from the order of the Heteroptera: for example Eurygaster spp., Stephanitis spp., Lygus spp., Aelia spp., Eurydema spp., Dysdercus spp., Piesma spp.
from the order of the Homoptera: for example Aleurodes spp., Bemisia spp., Trialeurodes spp., Brevicoryne spp., Cryptomyzus spp., Aphis spp., Eriosoma spp., Hyalopterus spp., Phylloxera spp., Pemphigus spp., Macrosiphum spp., Myzus spp., Phorodon spp., Rhopalosiphum spp., Empoasca spp., Euscelis spp., Eulecanium spp., Saissetia spp., Aonidiella spp., Aspidiotus spp., Nephotettix spp., Laodelphax spp., Nilaparvata spp., Sogatella spp., Pseudococcus spp., Psylla spp., Aphrophora spp., Aeneolamia spp., Aphidina ssp.,
from the order of the Lepidoptera: for example Pectinophora spp., Bupalus spp., Chematobia spp., Cnephasia spp., Hydraecia spp., Lithocolletis spp., Hyponomeuta spp., Plutella spp., Plutella xylostella, Malacosoma spp., Euproctis spp., Lymantria spp., Bucculatrix spp., Phytometra spp., Scrobipalpa spp., Phthorimaea spp., Gnorimoschema spp., Autographa spp., Evergestis spp., Lacanobia spp., Cydia spp., Pseudociaphila spp., Phyllocnistis spp., Agrotis spp., Euxoa spp., Feltia spp., Earias spp., Heliothis spp., Helicoverpa spp., Bombyx spp., Laphygma spp., Mamestra spp., Panolis spp., Prodenia spp., Spodoptera spp., Trichoplusia spp., Carpocapsa spp., Pieris spp., Chilo spp., Ostrinia spp., Pyrausta spp., Ephestia spp., Galleria spp., Cacoecia spp., Capua spp., Choristoneura spp., Clysia spp., Hofmannophila spp., Homona spp., Tineola spp., Tinea spp., Tortrix spp.
from the order of the Coleoptera: for example Anobium spp., Rhizopertha spp., Bruchidius spp., Acanthoscelides spp., Hylotrupes spp., Aclypea spp., Agelastica spp., Leptinotarsa spp., Psylliodes spp., Chaetocnema spp., Cassida spp., Bothynoderes spp., Clivina spp., Ceutorhynchus spp., Phyllotreta spp., Apion spp., Sitona spp., Bruchus spp., Phaedon spp., Diabrotica spp., Psylloides spp., Epilachna spp., Atomaria spp., Oryzaephilus spp., Anthonomus spp., Sitophilus spp., Otiorhynchus spp., Cosmopolites spp., Ceuthonynchus spp., Hypera spp., Dermestes spp., Trogoderma spp., Anthrenus spp., Attagenus spp., Lyctus spp., Meligethes spp., Ptinus spp., Niptus spp., Gibbium spp., Tribolium spp., Tenebrio spp., Agriotes spp., Conoderus spp., Melolontha spp., Amphimallon spp., Costelytra spp.
from the order of the Hymenoptera: for example Diprion spp., Hoplocampa spp., Lasius spp., Monomorium spp., Vespa spp.
from the order of the Diptera: for example Drosophila spp., Chrysomyxa spp., Hypoderma spp., Tannia spp., Bibio spp., Oscinella spp., Phorbia spp., Pegomyia spp., Ceratitis spp., Dacus spp., Tipula spp.
from the class of the Arachnida: for example Scorpio maurus, Latrodectus mactans.
from the order of the Acarina: for example Acarus spp., Bryobia spp., Panonychus spp., Tetranychus spp., Eotetranychus spp., Oligonychus spp., Eutetranychus spp., Eriophyes spp., Phyllocoptruta spp., Tarsonemus spp., Rhipicephalus spp., Rhipicephalus spp., Ixodes spp., Amblyomma spp.
from the class of the helminths: for example Meloidogyne spp., Heterodera spp., Globodera spp., Radopholus spp., Pratylenchus spp., Tylenchulus spp., Tylenchorhynchus spp., Rotylenchus spp., Heliocotylenchus spp., Belonoaimus spp., Longidorus spp., Trichodorus spp., Xiphinema spp., Ditylenchus spp., Aphelenchoides spp., Anguina spp.
from the class of the Gastropoda: for example Deroceras spp., Arion spp., Lymnaea spp., Galba spp., Succinea spp.
from the order of the Thysanoptera,
from the order of the Hemiptera: for example species of the sub-order Sternorrhyncha

The method according to the invention is preferably suitable for controlling Agriotes spp., Melolontha spp., Aphis spp., Cnephasia spp., Ostrinia spp., Agrotis spp., Hydraecia spp., Tipula spp., Myzus spp., Bemisia spp., Trialeurodes spp., Oscinella spp., Tetranychus spp., Lygus spp., Leptinotarsa spp., Psylliodes spp., Phytometra spp., Deroceras spp., Psylla spp., Blaniulus spp., Onychiurus spp., Piesma spp., Atomaria spp., Aclypea spp., Chaetocnema spp., Cassida spp., Bothynoderes spp., Clivina spp., Scrobipalpa spp., Phthorimaea spp., Gnorimoschema spp., Mamestra spp., Autographa spp., Arion spp., Gryllotalpa spp., Eurydema spp., Meligethes spp., Ceutorhynchus spp., Phyllotreta spp., Plutella xylostella, Evergestis spp., Lacanobia spp., Pieris spp., Forficula spp., Hypera spp., Apion spp., Otiorhynchus spp., Sitona spp., Acanthoscelides spp., Kakothrips spp., Bruchus spp., Cydia spp., Pseudociaphila spp., Heliothis spp., Helicoverpa spp., Prodenia spp., Spodoptera spp., Chilo spp and Diabrotica spp., Aphindina ssp., Frankliniella spp., Kakothrips spp., Hercinothrips spp., Scirtothrips spp., Taeniothrips spp., Thrips spp., Scorpio maurus, Latrodectus mactans.

The active compounds which can be used according to the invention are particularly suitable for controlling insects from the sub-order of the plant lice (Sternorrhyncha), in particular for controlling gall aphids (Phemphigidae), root aphids, jumping plant lice (Psyllidae), soft scales (Coccidae), armoured scales (Diaspididae), ensign coccids (Ortheziidae) or mealy-bugs (Pseudococcidae). This application is described in detail in WO 2006/077071, which document is incorporated herein by reference in this respect for the purpose of disclosure.

The method according to the invention is particularly suitable for treating Bt vegetables, Bt maize, Bt cotton, Bt soyabeans, Bt tobacco and also Bt rice, Bt sugar beet or Bt potatoes for controlling aphids (Aphidina), whiteflies (Trialeurodes), thrips (Thysanoptera), spider mites (Arachnida), scale insects and mealy-bugs (Coccoidae and Pseudococcoidae).

The active compounds which can be used according to the invention can be employed in customary formulations, such as solutions, emulsions, wettable powders, water- and oil-based suspensions, powders, dusts, pastes, soluble powders, soluble granules, granules for broadcasting, suspoemulsion concentrates, natural compounds impregnated with active compound, synthetic substances impregnated with active compound, fertilizers and also microencapsulations in polymeric substances.

These formulations are prepared in a known manner, for example by mixing the active compounds with extenders, i.e. liquid solvents and/or solid carriers, if appropriate using surfactants, i.e. emulsifiers and/or dispersants and/or foam-formers. The formulations are prepared either in suitable plants or else before or during application.

Wettable powders are preparations which can be dispersed homogeneously in water and which, in addition to the active compound and beside a diluent or inert substance, also comprise wetting agents, for example polyethoxylated alkylphenols, polyethoxylated fatty alcohols, alkylsulphonates or alkylphenylsulphonates and dispersants, for example sodium lignosulphonate, sodium 2,2′-dinaphthylmethane-6,6′-disulphonate.

Dusts are obtained by grinding the active compound with finely distributed solid substances, for example talc, natural clays, such as kaolin, bentonite, pyrophillite or diatomaceous earth. Granules can be prepared either by spraying the active compound onto granular inert material capable of adsorption or by applying active compound concentrates to the surface of carrier substances, such as sand, kaolinites or granular inert material, by means of adhesives, for example polyvinyl alcohol, sodium polyacrylate or mineral oils. Suitable active compounds can also be granulated in the manner customary for the preparation of fertilizer granules—if desired as a mixture with fertilizers.

Suitable for use as auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethyl sulphoxide, and also water.

Suitable solid carriers are:

for example, ammonium salts and ground natural minerals such as kaolins, clays, talc, chalk, quartz, attapulgite, montmorillonite or diatomaceous earth, and ground synthetic minerals, such as finely divided silica, alumina and silicates; suitable solid carriers for granules are: for example, crushed and fractionated natural rocks such as calcite, marble, pumice, sepiolite and dolomite, and also synthetic granules of inorganic and organic meals, and granules of organic material such as paper, sawdust, coconut shells, maize cobs and tobacco stalks; suitable emulsifiers and/or foam-formers are: for example, nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol-POE and/or -POP ethers, acid and/or POP POE esters, alkylaryl and/or POP POE ethers, fat and/or POP POE adducts, POE- and/or POP-polyol derivatives, POE- and/or POP-sorbitan or -sugar adducts, alkyl or aryl sulphates, alkyl- or arylsulphonates and alkyl or aryl phosphates or the corresponding PO-ether adducts. Furthermore, suitable oligo- or polymers, for example those derived from vinylic monomers, from acrylic acid, from EO and/or PO alone or in combination with, for example, (poly)alcohols or (poly)amines. It is also possible to employ lignin and its sulphonic acid derivatives, unmodified and modified celluloses, aromatic and/or aliphatic sulphonic acids and their adducts with formaldehyde.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

Other possible additives are perfumes, mineral or vegetable, optionally modified oils, waxes and nutrients (including trace nutrients), such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present.

These individual types of formulation are known in principle and are described, for example, in: Winnacker-Küchler, 1986, “Chemische Technologie” [Chemical Technology], Volume 7, 4th Ed., C. Hauser Verlag Munich; van Falkenberg, 1972-73, “Pesticides Formulations”, 2nd Ed., Marcel Dekker N.Y.; Martens, 1979, “Spray Drying Handbook”, 3rd Ed., G. Goodwin Ltd. London.

Based on his general expert knowledge, the person skilled in the art is able to choose suitable formulation auxiliaries (in this context, see, for example, Watkins, “Handbook of Insecticide Dust Diluents and Carriers”, 2nd Ed., Darland Books, Caldwell N.J.; v. Olphen, “Introduction to Clay Colloid Chemistry”, 2nd Ed., J. Wiley & Sons, N.Y.; Marsden, “Solvents Guide”, 2nd Ed., Interscience, N.Y. 1950; McCutcheon's, “Detergents and Emulsifiers Annual”, MC Publ. Corp., Ridgewood, N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Surface-active Ethylene Oxide Adducts], Wiss. Verlagsgesell., Stuttgart 1967; Winnacker-Küchler, “Chemische Technologie” [Chemical Technology], Volume 7, 4th Ed., C. Hanser Verlag Munich 1986.

In a preferred embodiment, the plants or plant parts are treated according to the invention with an oil-based suspension concentrate. An advantageous suspension concentrate is known from WO 2005/084435 (EP 1 725 104 A2). It consists of at least one agrochemically active compound solid at room temperature, at least one “closed” penetrant, at least one vegetable oil or mineral oil, at least one non-ionic surfactant and/or at least one anionic surfactant and, if appropriate, one or more additives from the groups of the emulsifiers, the antifoams, the preservatives, the antioxidants, the colourants and/or the inert fillers. Preferred embodiments of the suspension concentrate are described in the abovementioned WO 2005/084435. Corresponding suspension concentrates on a vegetable oil basis are described in EP 1 725 105 A2 expressly for the 3-APD which can be used here according to the invention. For the purpose of disclosure, both documents are incorporated herein in their entirety.

In a further preferred embodiment, the plants or plant parts are treated according to the invention with compositions comprising ammonium or phosphonium salts and, if appropriate, penetrants. An advantageous composition is known from DE 05059469. It consists of at least one active compound from the class of the 3-APD and at least one ammonium or phosphonium salt, and if appropriate penetrants. Preferred embodiments are described in DE 05059469. For the purpose of disclosure, this document is incorporated herein in its entirety.

In general, the formulations comprise from 0.01 to 98% by weight of active compound, preferably from 0.5 to 90%. In wettable powders, the active compound concentration is, for example, from about 10 to 90% by weight, the remainder to 100% by weight consisting of customary formulation components. In the case of emulsifiable concentrates, the active compound concentration can be from about 5 to 80% by weight. In most cases, formulations in the form of dusts comprise from 5 to 20% by weight of active compound, sprayable solutions comprise about 2 to 20% by weight. In the case of granules, the active compound content depends partially on whether the active compound is present in liquid or solid form and on which granulation auxiliaries, fillers, etc., are used.

The required application rate may also vary with external conditions such as, inter alia, temperature and humidity. It may vary within wide limits, for example between 0.1 g/h and 5.0 kg/ha or more of active substance. However, it is preferably between 0.1 g/ha and 1.0 kg/ha. Owing to the synergistic effects between Bt vegetable and insecticide, particular preference is given to application rates of from 0.1 to 500 g/ha.

For compounds of the formula (I), preference is given to application rates of from 10 to 500 g/ha, particular preference is given to 10 to 200 g/ha.

In their commercial formulations and in the use forms prepared from these formulations, the active compounds according to the invention may be present as mixtures with other active compounds, such as insecticides, attractants, sterilants, acaricides, nematicides, fungicides, growth-regulating substances or herbicides.

Particularly favourable mixing partners are, for example, the following compounds:

### Fungicides:

Inhibitors of nucleic acid synthesis
benalaxyl, benalaxyl-M, bupirimate, chiralaxyl, clozylacon, dimethirimol, ethirimol, furalaxyl, hymexazol, metalaxyl, metalaxyl-M, ofurace, oxadixyl, oxolinic acid
   Inhibitors of mitosis and cell division
benomyl, carbendazim, diethofencarb, fuberidazole, pencycuron, thiabendazole, thiophanat-methyl, zoxamide
   Inhibitors of respiratory chain complex I
diflumetorim
   Inhibitors of respiratory chain complex II
boscalid, carboxin, fenfuram, flutolanil, furametpyr, mepronil, oxycarboxin, penthiopyrad, thifluzamide
   Inhibitors of respiratory chain complex III
azoxystrobin, cyazofamid, dimoxystrobin, enestrobin, famoxadone, fenamidone, fluoxastrobin, kresoxim-methyl, metominostrobin, orysastrobin, pyraclostrobin, picoxystrobin, trifloxystrobin

### Decouplers

dinocap, fluazinam
   Inhibitors of ATP production
fentin acetate, fentin chloride, fentin hydroxide, silthiofam
   Inhibitors of amino acid biosynthesis and protein biosynthesis
andoprim, blasticidin-S, cyprodinil, kasugamycin, kasugamycin hydrochloride hydrate, mepanipyrim, pyrimethanil
   Inhibitors of signal transduction
fenpiclonil, fludioxonil, quinoxyfen
   Inhibitors of lipid and membrane synthesis
chlozolinate, iprodione, procymidone, vinclozolin
ampropylfos, potassium-ampropylfos, edifenphos, iprobenfos (IBP), isoprothiolane, pyrazophos
tolclofos-methyl, biphenyl
iodocarb, propamocarb, propamocarb hydrochloride
   Inhibitors of ergosterol biosynthesis
fenhexamid,
azaconazole, bitertanol, bromuconazole, cyproconazole, diclobutrazole, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, metconazole, myclobutanil, paclobutrazole, penconazole, propiconazole, prothioconazole, simeconazole, tebuconazole, tetraconazole, triadimefon, triadimenol, triticonazole, uniconazole, voriconazole, imazalil, imazalil sulphate, oxpoconazole, fenarimol, flurprimidole, nuarimol, pyrifenox, triforine, pefurazoate, prochloraz, triflumizole, viniconazole,
aldimorph, dodemorph, dodemorph acetate, fenpropimorph, tridemorph, fenpropidin, spiroxamine,
naftifine, pyributicarb, terbinafine
   Inhibitors of cell wall synthesis
benthiavalicarb, bialaphos, dimethomorph, flumorph, iprovalicarb, polyoxins, polyoxorim, validamycin A
   Inhibitors of melanin biosynthesis
capropamid, diclocymet, fenoxanil, phthalid, pyroquilon, tricyclazole
Resistance induction
acibenzolar-S-methyl, probenazole, tiadinil
Multisite
captafol, captan, chlorothalonil, copper salts such as: copper hydroxide, copper naphthenate, copper oxychloride, copper sulphate, copper oxide, oxine-copper and Bordeaux mixture, dichlofluanid, dithianon, dodine, dodine free base, ferbam, folpet, fluorofolpet, guazatine, guazatine acetate, iminoctadine, iminoctadine albesilate, iminoctadine triacetate, mancopper, mancozeb, maneb, metiram, metiram zinc, propineb, sulphur and sulphur preparations containing calcium polysulphide, thiram, tolylfluanid, zineb, ziram
Unknown mechanism
amibromdol, benthiazol, bethoxazin, capsimycin, carvone, quinomethionate, chloropicrin, cufraneb, cyflufenamid, cymoxanil, dazomet, debacarb, diclomezine, dichlorophen, dicloran, difenzoquat, difenzoquat methyl sulphate, diphenylamine, ethaboxam, ferimzone, flumetover, flusulphamide, fluopicolide, fluoroimide, hexachlorobenzene, 8-hydroxy-quinoline sulphate, irumamycin, methasulphocarb, metrafenone, methyl isothiocyanate, mildiomycin, natamycin, nickel dimethyl dithiocarbamate, nitrothal-isopropyl, octhilinone, oxamocarb, oxyfenthiin, pentachlorophenol and salts, 2-phenylphenol and salts, piperalin, propanosine-sodium, proquinazid, pyrrol nitrin, quintozene, tecloftalam, tecnazene, triazoxide, trichlamide, zarilamid and 2,3,5,6-tetrachloro-4-(methylsulphonyl)pyridine, N-(4-chloro-2-nitrophenyl)-N-ethyl-4-methylbenzenesulphonamide, 2-amino-4-methyl-N-phenyl-5-thiazolecarboxamide, 2-chloro-N-(2,3-dihydro-1,1,3-trimethyl-1H-inden-4-yl)-3-pyridinecarboxamide, 3-[5-(4-chlorophenyl)-2,3-dimethylisoxazolidin-3-yl]pyridine, cis-1-(4-chlorophenyl)-2-(1H-1,2,4-triazol-1-yl)cycloheptanol, 2,4-dihydro-5-methoxy-2-methyl-4-[[[[1-[3-(trifluoromethyl)phenyl]ethylidene]amino]oxy]methyl]phenyl]-3H-1,2,3-triazol-3-one (185336-79-2), methyl 1-(2,3-dihydro-2,2-dimethyl-1H-inden-1-yl)-1H-imidazole-5-carboxylate, 3,4,5-trichloro-2,6-pyridinedicarbonitrile, methyl 2-[[[cyclopropyl[(4-methoxyphenyl)imino]methyl]thio]methyl],alpha.-(methoxymethylene)benzacetate, 4-chloro-alpha-propynyloxy-N-[2-[3-methoxy-4-(2-propynyloxy)phenyl]ethyl]benz-acetamide, (2S)—N-[2-[4-[[3-(4-chlorophenyl)-2-propynyl]oxy]-3-methoxyphenyl]ethyl]-3-methyl-2-[(methylsulphonyl)amino]butanamide, 5-chloro-7-(4-methylpiperidin-1-yl)-6-(2,4,6-trifluorophenyl)[1,2,4]triazolo[1,5-a]pyrimidine, 5-chloro-6-(2,4,6-trifluorophenyl)-N-[(1R)-1,2,2-trimethylpropyl][1,2,4]triazolo[1,5-a]pyrimidin-7-amine, 5-chloro-N-[(1R)-1,2-dimethylpropyl]-6-(2,4,6-trifluorophenyl)[1,2,4]triazolo[1,5-a]pyrimidin-7-amine, N-[1-(5-bromo-3-chloropyridin-2-yl)ethyl]-2,4-dichloronicotinamide, N-(5-bromo-3-chloro-pyridin-2-yl)methyl-2,4-dichloronicotinamide, 2-butoxy-6-iodo-3-propylbenzopyranon-4-one, N-{(Z)-[(cyclopropylmethoxy)imino][6-(difluoromethoxy)-2,3-difluorophenyl]-methyl}-2-benzacetamide, N-(3-ethyl-3,5,5-trimethylcyclohexyl)-3-formylamino-2-hydroxybenzamide, 2-[[[[1-[3(1-fluoro-2-phenylethyl)oxy]phenyl]ethylidene]amino]oxy]-methyl]-alpha-(methoxyimino)-N-methyl-alphaE-benzacetamide, N-{2-[3-chloro-5-(trifluoromethyl)pyridin-2-yl]ethyl}-2-(trifluoromethyl)benzamide, N-(3′,4′-dichloro-5-fluorobiphenyl-2-yl)-3-(difluoromethyl)-1-methyl-1H-pyrazole-4-carboxamide, N-(6-methoxy-3-pyridinyl)cyclopropanecarboxamide, 1-[(4-methoxyphenoxy)methyl]-2,2-dimethylpropyl-1H-imidazole-1-carboxylic acid, O-[1-[4-methoxyphenoxy)methyl]-2,2-dimethylpropyl]-1H-imidazole-1-carbothioic acid, 2-(2-{[6-(3-chloro-2-methylphenoxy)-5-fluoropyrimidin-4-yl]oxy}phenyl)-2-(methoxyimino)-N-methylacetamide

### Bactericides:

bronopol, dichlorophen, nitrapyrin, nickel dimethyldithiocarbamate, kasugamycin, octhilinone, furancarboxylic acid, oxytetracycline, probenazole, streptomycin, tecloftalam, copper sulphate and other copper preparations.

### Insecticides/acaricides/nematicides:

Acetylcholine esterase (AChE) inhibitors
   carbamates,
   for example alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulphan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb, triazamate
   organophosphates,
   for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl, -ethyl), coumaphos, cyanofenphos, cyanophos, chlorfenvinphos, demeton-S-methyl, demeton-S-methylsulphone, dialifos, diazinon, dichlofenthion, dichlorvos/DDVP, dicrotophos, dimethoate, dimethylvinphos, dioxabenzofos, disulphoton, EPN, ethion, ethoprophos, etrimfos, famphur, fenamiphos, fenitrothion, fensulphothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulphotep, sulprofos, tebupirimfos, temephos, terbufos, tetrachlorvinphos, thiometon, triazophos, triclorfon, vamidothion
Sodium channel modulators/voltage-dependent sodium channel blockers
   pyrethroids,
   for example acrinathrin, allethrin (d-cis-trans, d-trans), beta-cyfluthrin, bifenthrin, bioallethrin, bioallethrin-S-cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans-isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum)
DDT
   oxadiazines,
   for example indoxacarb
   semicarbazones,
   for example metaflumizone (BAS3201)
Acetylcholine receptor agonists/antagonists
   chloronicotinyls,
   for example acetamiprid, clothianidin, dinotefuran, imidacloprid, nitenpyram, nithiazine, thiacloprid, thiamethoxam
nicotine, bensultap, cartap
Acetylcholine receptor modulators
   spinosyns,
   for example spinosad,
GABA-controlled chloride channel antagonists
   organochlorines,
   for example camphechlor, chlordane, endosulphan, gamma-HCH, HCH, heptachlor, lindane, methoxychlor
   fiprols,
   for example acetoprole, ethiprole, fipronil, pyrafluprole, pyriprole, vaniliprole
Chloride channel activators
   mectins,
   for example abarmectin, emamectin, emamectin-benzoate, ivermectin, lepimectin, milbemycin
   Juvenile hormone mimetics,
   for example diofenolan, epofenonane, fenoxycarb, hydroprene, kinoprene, methoprene, pyriproxifen, triprene
Ecdysone agonists/disruptors
   diacylhydrazines,
   for example chromafenozide, halofenozide, methoxyfenozide, tebufenozide
Chitin biosynthesis inhibitors
   benzoylureas,
   for example bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron
   buprofezin
   cyromazine
Oxidative phosphorylation inhibitors, ATP disruptors
   diafenthiuron
   organotin compounds,
   for example azocyclotin, cyhexatin, fenbutatin-oxide
Oxidative phosphorylation decouplers acting by interrupting the H-proton gradient
   pyrroles,
   for example chlorfenapyr
   dinitrophenols,
   for example binapacyrl, dinobuton, dinocap, DNOC, meptyldinocap
Site-I electron transport inhibitors
   METIs,
   for example fenazaquin, fenpyroximate, pyrimidifen, pyridaben, tebufenpyrad,
   tolfenpyrad
   hydramethylnon
   dicofol
Site-II electron transport inhibitors
   rotenone
Site-III electron transport inhibitors
   acequinocyl, fluacrypyrim
Microbial disruptors of the insect gut membrane
   Bacillus thuringiensis strains
Lipid synthesis inhibitors
   tetronic acids,
   for example spirodiclofen, spiromesifen
   tetramic acids,
   for example cis-3-(2,5-dimethylphenyl)-4-hydroxy-8-methoxy-1-azaspiro[4.5]-dec-3-en-2-one
   carboxamides,
   for example flonicamid
   octopaminergic agonists,
   for example amitraz
Inhibitors of magnesium-stimulated ATPase,
   propargite
   nereistoxin analogues,
   for example thiocyclam hydrogen oxalate, thiosultap-sodium
Ryanodin receptor agonists
   benzoic acid dicarboxamides,
   for example flubendiamid
   anthranilamides,
   for example rynaxypyr (3-bromo-N-{4-chloro-2-methyl-6-[(methylamino)-carbonyl]phenyl}-1-(3-chloropyridin-2-yl)-1H-pyrazole-5-carboxamide)
Biologicals, hormones or pheromones
   azadirachtin, Bacillus spec., Beauveria spec., codlemone, Metarrhizium spec., Paecilomyces spec., thuringiensin, Verticillium spec.
Active compounds with unknown or unspecific mechanisms of action
   fumigants,
   for example aluminium phosphide, methyl bromide, sulphuryl fluoride
   antifeedants,
   for example cryolite, flonicamid, pymetrozine
   mite growth inhibitors,
   for example clofentezine, etoxazole, hexythiazox amidoflumet, benclothiaz, benzoximate, bifenazate, bromopropylate, buprofezin, chinomethionat, chlordimeform, chlorobenzilate, chloropicrin, clothiazoben, cycloprene, cyflumetofen, dicyclanil, fenoxacrim, fentrifanil, flubenzimine, flu-fenerim, flutenzin, gossyplure, hydramethylnone, japonilure, metoxadiazone, petroleum, piperonyl butoxide, potassium oleate, pyridalyl, sulphluramid, tetra-difon, tetrasul, triarathene, verbutin

A mixture with other known active compounds, such as herbicides, fertilizers, growth regulators, safeners, semiochemicals, or else with agents for improving the plant properties, is also possible.

The active compound content of the use forms prepared from the commercially available formulations can be from 0.00000001 to 95% by weight, preferably between 0.00001 and 1% by weight, of active compound.

TABLE 1
Plant: Maize

| Structure affected or principle expressed | Feature of the plant/tolerance to |
|---|---|
| acetolactate synthase (ALS) | sulphonylurea compounds, imidazolinones |
| | triazolpyrimidines, pyrimidyloxybenzoates, |
| | phthalides |
| acetyl-CoA carboxylase (ACCase) | aryloxyphenoxyalkanecarboxylic acid, |
| | cyclohexanedione |
| hydroxyphenylpyruvate dioxygenase (HPPD) | isooxazoles, such as isoxaflutol or |
| | isoxachlortol, |
| | triones, such as mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles, such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazole, pyridine derivative, |
| | phenopylate, oxadiazoles etc. |
| cytochrome P450 e.g. P450 SU1 | xenobiotics and herbicides, such as |
| | sulphonylurea |
| dimboa biosynthesis (Bx1-Gen) | Helminthosporium turcicum, |
| | Rhopalosiphum maydis, Diplodia |
| | maydis, Ostrinia nubilalis, Lepidoptera sp. |
| CMIII (small basic peptide building block | plant pathogens e.g. Fusarium, Alternaria, |
| from maize grain) | Sclerotina |
| Com-SAFP (zeamatin) | plant pathogens, e.g. Fusarium, |
| | Alternaria, Sclerotina, Rhizoctonia, |
| | Chaetomium, Phycomycen |
| Hm1-gene | Cochliobulus |
| chitinases | plant pathogens |
| glucanases | plant pathogens |
| envelope proteins | viruses, such as the Maize dwarf mosaic virus |
| | (MDMV) |
| toxins of Bacillus thuringiensis, VIP 3, | Lepidoptera, Coleoptera, Diptera, |
| Bacillus cereus toxin, Photorabdus and | nematodes, e.g. Ostrinia nubilalis, |
| Xenorhabdus toxins | Heliothis zea, armyworms e.g. |
| | Spodoptera frugiperda, Western corn |
| | rootworm, Sesamia sp., Aprotis ipsilon, Asian |
| | corn borer, weevils |
| 3-hydroxysteroid oxidase | Lepidoptera, Coleoptera, Diptera, nematodes, |
| | e.g. Ostrinia nubilalis, Heliothis zea, |
| | armyworms e.g. Spodoptera frugiperda, |
| | Western corn rootworm, Sesamia sp., Aprotis |
| | ipsilon, |
| | Asian corn borer, weevils |
| peroxidase | Lepidoptera, Coleoptera, Diptera, nematodes, |
| | e.g. Ostrinia nubilalis, Heliothis zea, |
| | armyworms e.g. Spodoptera frugiperda, |
| | Western corn rootworm, Sesamia sp., Aprotis |
| | ipsilon, Asian corn borer, weevils |
| aminopeptidase inhibitors, e.g. leucine | Lepidoptera, Coleoptera, Diptera, |
| aminopeptidase inhibitors (LAPI) | nematodes, e.g. Ostrinia nubilalis, |
| | Heliothis zea, armyworms e.g. Spodoptera |
| | frugiperda, Western corn rootworm, Sesamia |
| | sp., Aprotis ipsilon, Asian corn borer, weevils |
| limonene synthase | Western corn rootworm |
| lectin | Lepidoptera, Coleoptera, Diptera, nematodes, |
| | e.g. Ostrinia nubilalis, Heliothis zea, |
| | armyworms e.g. Spodoptera frugiperda, |
| | Western corn rootworm, Sesamia sp., Aprotis |
| | ipsilon, Asian corn borer, weevils |
| protease inhibitors e.g. cystatin, patatin, | weevils, Western corn rootworm |
| virgiferin, CPTI | |
| ribosome-inactivating protein | Lepidoptera, Coleoptera, Diptera, nematodes, |
| | e.g. Ostrinia nubilalis, Heliothis zea, |
| | armyworms e.g. Spodoptera frugiperda, |
| | Western corn rootworm, Sesamia sp., Aprotis |
| | ipsilon, Asian corn borer, weevils |
| 5C9-maize polypeptide | Lepidoptera, Coleoptera, Diptera, nematodes, |
| | e.g. Ostrinia nubilalis, Heliothis zea, |
| | armyworms e.g. Spodoptera frugiperda, |
| | Western corn rootworm, Sesamia sp., Aprotis |
| | ipsilon, Asian corn borer, weevils |
| HMG-CoA reductase | Lepidoptera, Coleoptera, Diptera, nematodes, |
| | e.g. Ostrinia nubilalis, Heliothis zea, |
| | armyworms e.g. Spodoptera frugiperda, |
| | Western corn rootworm, Sesamia sp., Aprotis |
| | ipsilon, Asian corn borer, weevils |

Plant: Wheat

| Structure affected/protein expressed | Feature of the plant/tolerance to |
|---|---|
| acetolactate synthase (ALS) | sulphonylurea compods, imidazolinones |
| | triazolpyrimidines, pyrimidyloxybenzoates, |
| | phthalides |
| acetyl-CoA carboxylase (ACCase) | aryloxyphenoxyalkanecarboxylic acid, |
| | cyclohexanedione |
| hydroxyphenylpyruvate dioxygenase (HPPD) | isooxazoles, such as isoxaflutol |
| | or isoxachlortol, |
| | triones, such as mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles, such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazoles, pyridine derivatives, |
| | phenopylate, oxadiazoles etc. |
| cytochrome P450 e.g. P450 SU1 | xenobiotics and herbicides, such as |
| | sulphonylurea compounds |
| antifungal polypeptide AlyAFP | plant pathogens, e.g. Septoria and Fusarium |
| glucose oxidase | plant pathogens, e.g. Fusarium, Septoria |
| pyrrolnitrin synthesis gene | plant pathogens, e.g. Fusarium, Septoria |
| serine/threonine kinases | plant pathogens, e.g. Fusarium, Septoria |
| | and other diseases |
| polypeptide having the effect of triggering | plant pathogens, e.g. Fusarium, Septoria and |
| a hypersensitivity reaction | other diseases |
| systemic aquired resistance (SAR) genes | viral, bacterial, fungal and nematodal |
| | pathogens |
| chitinases | plant pathogens |
| glucanases | plant pathogens |
| double-strand ribonuclease | viruses such as, for example, BYDV and |
| | MSMV |
| envelope proteins | viruses such as, for example, BYDV and |
| | MSMV |
| toxins of Bacillus thuringiensis, VIP 3, | Lepidoptera, Coleoptera, Diptera, |
| Bacillus cereus toxins, Photorabdus and | nematodes |
| Xenorhabdus toxins | |
| 3-hydroxysteroid oxidase | Lepidoptera, Coleoptera, Diptera, |
| | nematodes |
| peroxidase | Lepidoptera, Coleoptera, Diptera, |
| | nematodes |
| aminopeptidase inhibitors, e.g. leucine | Lepidoptera, Coleoptera, Diptera, |
| aminopeptidase inhibitor | nematodes |
| lectins | Lepidoptera, Coleoptera, Diptera, |
| | nematodes, aphids |
| protease inhibitors, e.g. cystatin, patatin, | Lepidoptera, Coleoptera, Diptera, |
| virgiferin, CPTI | nematodes, aphids |
| ribosome-inactivating protein | Lepidoptera, Coleoptera, Diptera, |
| | nematodes, aphids |
| HMG-CoA reductase | Lepidoptera, Coleoptera, Diptera, |
| | nematodes, e.g. Ostrinia nubilalis, |
| | Heliothis zea, armyworms e.g. Spodoptera |
| | frugiperda, Western corn rootworm, Sesamia |
| | sp., Aprotis ipsilon, Asian corn borer, weevils |

Plant: Barley

| Structure affected/protein expressed | Feature of the plant/tolerance to |
|---|---|
| acetolactate synthase (ALS) | sulphonylurea compounds, imidazolinones |
| | triazolpyrimidines, pyrimidyloxybenzoates, |
| | phthalides |
| acetyl-CoA carboxylase (ACCase) | aryloxyphenoxyalkanecarboxylic acids, |
| | cyclohexanediones |
| hydroxyphenylpyruvate dioxygenase (HPPD) | isooxazoles, such as isoxaflutol or |
| | isoxachlortol, |
| | triones, such as mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles, such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazoles, pyridine derivatives, |
| | phenopylate, oxadiazoles etc. |
| cytochrome P450 e.g. P450 SU1 | xenobiotics and herbicides, such as |
| | sulphonylurea compounds |
| antifungal polypeptide AlyAFP | plant pathogens, e.g. Septoria and Fusarium |
| glucose oxidase | plant pathogens, e.g. Fusarium, Septoria |
| pyrrolnitrin synthesis gene | plant pathogens, e.g. Fusarium, Septoria |
| serine/threonine kinases | plant pathogens, e.g. Fusarium, Septoria |
| | and other diseases |
| polypeptide having the effect of triggering | plant pathogens, e.g. Fusarium, Septoria and |
| a hypersensitivity reaction | other diseases |
| systemic aquired resistance (SAR) genes | viral, bacterial, fungal and nematodal |
| | pathogens |
| chitinases | plant pathogens |
| glucanases | plant pathogens |
| double-strand ribonuclease | viruses such as, for example, BYDV and |
| | MSMV |
| envelope proteins | viruses such as, for example, BYDV and |
| | MSMV |
| toxins of Bacillus thuringiensis, VIP 3, | Lepidoptera, Coleoptera, Diptera, |
| Bacillus cereus toxins, Photorabdus and | nematodes |
| Xenorhabdus toxins | |
| 3-hydroxysteroid oxidase | Lepidoptera, Coleoptera, Diptera, |
| | nematodes |
| peroxidase | Lepidoptera, Coleoptera, Diptera, |
| | nematodes |
| aminopeptidase inhibitors, e.g. leucine | Lepidoptera, Coleoptera, Diptera, |
| aminopeptidase inhibitor | nematodes |
| lectins | Lepidoptera, Coleoptera, Diptera, |
| | nematodes, aphids |
| protease inhibitors, e.g. cystatin, patatin, | Lepidoptera, Coleoptera, Diptera, |
| virgiferin, CPTI | nematodes, aphids |
| ribosome-inactivating protein | Lepidoptera, Coleoptera, Diptera, |
| | nematodes, aphids |
| HMG-CoA reductase | Lepidoptera, Coleoptera, Diptera, |
| | nematodes, aphids |

Plant: Rice

| Structure affected/principle expressed | Feature of the plant/tolerance to |
|---|---|
| acetolactate synthase (ALS) | sulphonylurea compounds, imidazolinones |
| | triazolpyrimidines, pyrimidyloxybenzoates, |
| | phthalides |
| acetyl-CoA carboxylase (ACCase) | aryloxyphenoxyalkanecarboxylic acid, |
| | cyclohexanedione |
| hydroxyphenylpyruvate dioxygenase (HPPD) | isooxazoles, such as isoxaflutol or |
| | isoxachlortol, |
| | triones, such as mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles, such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazoles, pyridine derivatives, |
| | phenopylate, oxadiazoles etc. |
| cytochrome P450 e.g. P450 SU1 | xenobiotics and herbicides, such as |
| | sulphonylurea compounds |
| antifungal polypeptide AlyAFP | plant pathogens |
| glucose oxidase | plant pathogens |
| pyrrolnitrin synthesis gene | plant pathogens |
| serine/threonine kinases | plant pathogens |
| phenylalanine ammonia lyase (PAL) | plant pathogens, e.g. bacterial |
| | foliar mildew and inducible rice blast |
| phytoalexins | plant pathogens, e.g. bacterial |
| | foliar mildew and rice blast |
| B-1,3-glucanase (antisense) | plant pathogens, e.g. bacterial |
| | foliar mildew and rice blast |
| receptor kinase | plant pathogens, e.g. bacterial |
| | foliar mildew and rice blast |
| polypeptide having the effect of triggering | plant pathogens |
| a hypersensitivity reaction | |
| systemic aquired resistance (SAR) genes | viral, bacterial, fungal and nematodal |
| | pathogens |
| chitinases | plant pathogens, e.g. bacterial |
| | foliar mildew and rice blast |
| glucanases | plant pathogens |
| double-strand ribonuclease | viruses such as, for example, BYDV and |
| | MSMV |
| envelope proteins | viruses such as, for example, BYDV and |
| | MSMV |
| toxins of Bacillus thuringiensis, VIP 3, | Lepidoptera, e.g. stem borer, Coleoptera, |
| Bacillus cereus toxins, Photorabdus and | e.g. weevils such as Lissorhoptrus oryzophilus, |
| Xenorhabdus toxins | Diptera, rice planthoppers, e.g. rice brown |
| | planthopper |
| 3-hydroxysteroid oxidase | Lepidoptera, e.g. stem borer, Coleoptera, |
| | e.g. weevils such as Lissorhoptrus oryzophilus, |
| | Diptera, rice planthoppers, e.g. rice brown |
| | planthopper |
| peroxidase | Lepidoptera, e.g. stem borer, Coleoptera, |
| | e.g. weevils such as Lissorhoptrus oryzophilus, |
| | Diptera, rice planthoppers, e.g. rice brown |
| | planthopper |
| aminopeptidase inhibitors, e.g. leucine | Lepidoptera, e.g. stem borer, Coleoptera, |
| aminopeptidase inhibitor | e.g. weevils such as Lissorhoptrus |
| | oryzophilus, Diptera, rice planthoppers, e.g. |
| | rice brown planthopper |
| lectins | Lepidoptera, e.g. stem borer, Coleoptera, |
| | e.g. weevils such as Lissorhoptrus |
| | oryzophilus, Diptera, rice planthoppers, e.g. |
| | rice brown planthopper |
| protease inhibitors | Lepidoptera, e.g. stem borer, Coleoptera, |
| | e.g. weevils such as Lissorhoptrus oryzophilus, |
| | Diptera, rice planthoppers e.g. rice brown |
| | planthopper |
| ribosome-inactivating protein | Lepidoptera, e.g. stem borer, Coleoptera, |
| | e.g. weevils such as Lissorhoptrus |
| | oryzophilus, Diptera, rice planthoppers, e.g. |
| | rice brown planthopper |
| HMG-CoA reductase | Lepidoptera, e.g. stem borer, Coleoptera, |
| | e.g. weevils such as Lissorhoptrus |
| | oryzophilus, Diptera, rice planthoppers e.g. |
| | rice brown planthopper |

Plant: Soya bean

| Structure affected/principle expressed | Feature of the plant/tolerance to |
|---|---|
| acetolactate synthase (ALS) | sulphonylurea compounds, imidazolinones |
| | triazolpyrimidines, pyrimidyloxybenzoates, |
| | phthalides |
| acetyl-CoA carboxylase (ACCase) | aryloxyphenoxyalkanecarboxylic acids, |
| | cyclohexanediones |
| hydroxyphenylpyruvate dioxygenase (HPPD) | isooxazoles, such as isoxaflutol or |
| | isoxachlortol, |
| | triones, such as mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles, such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphonate |
| protoporphyrinogen oxidase (PROTOX) | diphenyl ethers, cyclic imides, |
| | phenylpyrazoles, pyridine derivatives, |
| | phenopylate, oxadiazoles etc. |
| cytochrome P450 e.g. P450 SU1 or selection | xenobiotics and herbicides, such as |
| | sulphonylurea compounds |
| antifungal polypeptide AlyAFP | bacterial and fungal pathogens such as, for |
| | example, Fusarium, Sclerotinia, stem rot |
| oxalate oxidase | bacterial and fungal pathogens such as, for |
| | example, Fusarium, Sclerotinia, stem rot |
| glucose oxidase | bacterial and fungal pathogens such as, for |
| | example, Fusarium, Sclerotinia, stem rot |
| pyrrolnitrin synthesis gene | bacterial and fungal pathogens such as, for |
| | example, Fusarium, Sclerotinia, stem rot |
| serine/threonine kinases | bacterial and fungal pathogens such as, for |
| | example, Fusarium, Sclerotinia, stem rot |
| phenylalanine ammonia lyase (PAL) | bacterial and fungal pathogens such as, for |
| | example, Fusarium, Sclerotinia, stem rot |
| phytoalexins | plant pathogens, e.g. bacterial foliar |
| | mildew and rice blast |
| B-1,3-glucanase (antisense) | plant pathogens, e.g. bacterial foliar |
| | mildew and rice blast |
| receptor kinase | bacterial and fungal pathogens such as, for |
| | example, Fusarium, Sclerotinia, stem rot |
| polypeptide having the effect of triggering | plant pathogens |
| a hypersensitivity reaction | |
| systemic aquired resistance (SAR) genes | viral, bacterial, fungal and nematodal |
| | pathogens |
| chitinases | bacterial and fungal pathogens such as, for |
| | example, Fusarium, Sclerotinia, stem rot |
| glucanases | bacterial and fungal pathogens such as, for |
| | example, Fusarium, Sclerotinia, stem rot |
| double-strand ribonuclease | viruses such as, for example, BPMV and |
| | SbMV |
| envelope proteins | viruses such as, for example, BYDV and |
| | MSMV |
| toxins of Bacillus thuringiensis, VIP 3, | Lepidoptera, Coleoptera, aphids |
| Bacillus cereus toxins, Photorabdus and | |
| Xenorhabdus toxins | |
| 3-hydroxysteroid oxidase | Lepidoptera, Coleoptera, aphids |
| peroxidase | Lepidoptera, Coleoptera, aphids |
| aminopeptidase inhibitors, e.g. leucine | Lepidoptera, Coleoptera, aphids |
| aminopeptidase inhibitor | |
| lectins | Lepidoptera, Coleoptera, aphids |
| protease inhibitors, e.g. virgiferin | Lepidoptera, Coleoptera, aphids |
| ribosome-inactivating protein | Lepidoptera, Coleoptera, aphids |
| HMG-CoA reductase | Lepidoptera, Coleoptera, aphids |
| barnase | nematodes, e.g. root-knot nematodes and |
| | cyst nematodes |
| hatching factor for cyst nematodes | cyst nematodes |
| principles for preventing food uptake | nematodes, e.g. root-knot nematodes and |
| | cyst nematodes |

Plant: Potato

| Structure affected/protein expressed | Feature of the plant/tolerance to |
|---|---|
| acetolactate synthase (ALS) | sulphonylurea compounds, imidazolinones |
| | triazolpyrimidines, pyrimidyloxybenzoates, |
| | phthalides |
| acetyl-CoA carboxylase (ACCase) | aryloxyphenoxyalkanecarboxylic acids, |
| | cyclohexanediones |
| hydroxyphenylpyruvate dioxygenase (HPPD) | isooxazoles, such as isoxaflutol or |
| | isoxachlortol, |
| | triones, such as mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles, such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazoles, pyridine derivatives, |
| | phenopylate, oxadiazoles etc. |
| cytochrome P450 e.g. P450 SU1 or selection | xenobiotics and herbicides, such as |
| | sulphonylurea compounds |
| polyphenol oxidase or polyphenol oxidase | black spot |
| (antisense) | |
| metallothionein | bacterial and fungal pathogens such as, for |
| | example, Phytophtora, |
| ribonuclease | Phytophtora, Verticillium, Rhizoctonia |
| antifungal polypeptide AlyAFP | bacterial and fungal pathogens such as, for |
| | example, Phytophtora |
| oxalate oxidase | bacterial and fungal pathogens such as, for |
| | example, Phytophtora, Verticillium, Rhizoctonia |
| glucose oxidase | bacterial and fungal pathogens such as, for |
| | example, Phytophtora, Verticillium, Rhizoctonia |
| pyrrolnitrin synthesis gene | bacterial and fungal pathogens such as, for |
| | example, Phytophtora, Verticillium, Rhizoctonia |
| serine/threonine kinases | bacterial and fungal pathogens such as, for |
| | example, Phytophtora, Verticillium, Rhizoctonia |
| cecropin B | bacteria such as, for example, Coryne- |
| | bacterium sepedonicum, Erwinia carotovora |
| phenylalanine ammonia lyase (PAL) | bacterial and fungal pathogens such as, for |
| | example, Phytophtora, Verticillium, Rhizoctonia |
| phytoalexins | bacterial and fungal pathogens such as, for |
| | example, Phytophtora, Verticillium, Rhizoctonia |
| B-1,3-glucanase (antisense) | bacterial and fungal pathogens such as, for |
| | example, Phytophtora, Verticillium, Rhizoctonia |
| receptor kinase | bacterial and fungal pathogens such as, for |
| | example, Phytophtora, Verticillium, Rhizoctonia |
| polypeptide having the effect of triggering | bacterial and fungal pathogens such as, for |
| a hypersensitivity reaction | example, Phytophtora, Verticillium, Rhizoctonia |
| systemic aquired resistance (SAR) genes | viral, bacterial, fungal and nematodal |
| | pathogens |
| chitinases | bacterial and fungal pathogens such as, for |
| | example, Phytophtora, Verticillium, Rhizoctonia |
| barnase | bacterial and fungal pathogens such as, for |
| | example, Phytophtora, Verticillium, Rhizoctonia |
| gene 49 for controlling disease resistance | bacterial and fungal pathogens such as, for |
| | example, Phytophtora, Verticillium, Rhizoctonia |
| trans-aldolase (antisense) | black spot |
| glucanases | bacterial and fungal pathogens such as, for |
| | example, Phytophtora, Verticillium, Rhizoctonia |
| double-strand ribonuclease | viruses such as, for example, PLRV, PVY and |
| | TRV |
| envelope proteins | viruses such as, for example, PLRV, PVY and |
| | TRV |
| 17 kDa or 60 kDa protein | viruses such as, for example, PLRV, PVY and |
| | TRV |
| nuclear inclusion proteins, e.g. a or b | viruses such as, for example, PLRV, PVY and |
| | TRV |
| pseudoubiquitin | viruses such as, for example, PLRV, PVY and |
| | TRV |
| replicase | viruses such as, for example, PLRV, PVY and |
| | TRV |
| toxins of Bacillus thuringiensis, VIP 3, | Coleoptera, e.g. Colorado beetle, aphids |
| Bacillus cereus toxins, Photorabdus and | |
| Xenorhabdus toxins | |
| 3-hydroxysteroid oxidase | Coleoptera, e.g. Colorado beetle, aphids |
| peroxidase | Coleoptera, e.g. Colorado beetle, aphids |
| aminopeptidase inhibitors, e.g. leucine | Coleoptera, e.g. Colorado beetle, aphids |
| aminopeptidase inhibitor | |
| stilbene synthase | Coleoptera, e.g. Colorado beetle, aphids |
| lectins | Coleoptera, e.g. Colorado beetle, aphids |
| protease inhibitors, e.g. cystatin, patatin | Coleoptera, e.g. Colorado beetle, aphids |
| ribosomene-inactivating protein | Coleoptera, e.g. Colorado beetle, aphids |
| HMG-CoA reductase | Coleoptera, e.g. Colorado beetle, aphids |
| hatching factor for cyst nematodes | cyst nematodes |
| barnase | nematodes, e.g. root-knot nematodes and |
| | cyst nematodes |
| principles for preventing food uptake | nematodes, e.g. root-knot nematodes and |
| | cyst nematodes |

Plant: Tomato

| Structure affected/principle expressed | Feature of the plant/tolerance to |
|---|---|
| acetolactate synthase (ALS) | sulphonylurea compounds, imidazolinones |
| | triazolpyrimidines, pyrimidyloxybenzoates, |
| | phthalides |
| acetyl-CoA carboxylase (ACCase) | aryloxyphenoxyalkanecarboxylic acid, |
| | cyclohexanedione |
| hydroxyphenylpyruvate dioxygenase (HPPD) | isooxazoles, such as isoxaflutol or |
| | isoxachlortol, |
| | triones, such as mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles, such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazoles, pyridine derivatives, |
| | phenopylate, oxadiazoles etc. |
| Cytochrome P450 e.g. P450 SU1 or selection | xenobiotics and herbicides, such as |
| | sulphonylurea compounds |
| polyphenol oxidase or polyphenol oxidase | black spot |
| (antisense) | |
| metallothionein | bacterial and fungal pathogens such as, for |
| | example, Phytophtora |
| ribonuclease | Phytophtora, Verticillium, Rhizoctonia |
| antifungal polypeptide AlyAFP | bacterial and fungal pathogens such as, for |
| | example, bacterial blotch, Fusarium, |
| | soft rot, powdery mildew, foliar blight, |
| | leaf mould etc. |
| oxalate oxidase | bacterial and fungal pathogens such as, for |
| | example, bacterial blotch, Fusarium, |
| | soft rot, powdery mildew, foliar blight, |
| | leaf mould etc. |
| glucose oxidase | bacterial and fungal pathogens such as, for |
| | example, bacterial blotch, Fusarium, |
| | soft rot, powdery mildew, foliar blight, |
| | leaf mould etc. |
| pyrrolnitrin synthesis gene | bacterial and fungal pathogens such as, for |
| | example, bacterial blotch, Fusarium, |
| | soft rot, powdery mildew, foliar blight, |
| | leaf mould etc. |
| serine/threonine kinases | bacterial and fungal pathogens such as, for |
| | example, bacterial blotch, Fusarium, |
| | soft rot, powdery mildew, foliar blight, |
| | leaf mould etc. |
| cecropin B | bacterial and fungal pathogens such as, for |
| | example, bacterial blotch, Fusarium, |
| | soft rot, powdery mildew, foliar blight, |
| | leaf mould etc. |
| phenylalanine ammonia lyase (PAL) | bacterial and fungal pathogens such as, for |
| | example, bacterial blotch, Fusarium, |
| | soft rot, powdery mildew, foliar blight, |
| | leaf mould etc. |
| Cf genes, e.g. Cf 9 Cf5 Cf4 Cf2 | leaf mould |
| osmotin | early blight |
| alpha hordothionin | bakteria |
| systemin | bacterial and fungal pathogens such as, for |
| | example, bacterial blotch, Fusarium, |
| | soft rot, powdery mildew, foliar blight, |
| | leaf mould etc. |
| polygalacturonase inhibitors | bacterial and fungal pathogens such as, for |
| | example, bacterial blotch, Fusarium, |
| | soft rot, powdery mildew, foliar blight, |
| | leaf mould etc. |
| Prf control gene | bacterial and fungal pathogens such as, for |
| | example, bacterial blotch, Fusarium, |
| | soft rot, powdery mildew, foliar blight, |
| | leaf mould etc. |
| 12 fusarium resistance site | Fusarium |
| phytoalexins | bacterial and fungal pathogens such as, for |
| | example, bacterial blotch, Fusarium, |
| | soft rot, powdery mildew, foliar blight, |
| | leaf mould etc. |
| B-1,3-glucanase (antisense) | bacterial and fungal pathogens such as, for |
| | example, bacterial blotch, Fusarium, |
| | soft rot, powdery mildew, foliar blight, |
| | leaf mould etc. |
| receptor kinase | bacterial and fungal pathogens such as, for |
| | example, bacterial blotch, Fusarium, |
| | soft rot, powdery mildew, foliar blight, |
| | leaf mould etc. |
| polypeptide having the effect of triggering | bacterial and fungal pathogens such as, for |
| a hypersensitivity reaction | example, bacterial blotch, Fusarium, |
| | soft rot, powdery mildew, foliar blight, |
| | leaf mould etc. |
| systemic aquired resistance (SAR) genes | viral, bacterial, fungal and nematodal |
| | pathogens |
| chitinases | bacterial and fungal pathogens such as, for |
| | example, bacterial blotch, Fusarium, |
| | soft rot, powdery mildew, foliar blight, |
| | leaf mould etc. |
| barnase | bacterial and fungal pathogens such as, for |
| | example, bacterial blotch, Fusarium, |
| | soft rot, powdery mildew, foliar blight, |
| | leaf mould etc. |
| glucanases | bacterial and fungal pathogens such as, for |
| | example, bacterial blotch, Fusarium, |
| | soft rot, powdery mildew, foliar blight, |
| | leaf mould etc. |
| double-strand ribonuclease | viruses such as, for example, PLRV, PVY and |
| | ToMoV |
| envelope proteins | viruses such as, for example, PLRV, PVY and |
| | ToMoV |
| 17 kDa or 60 kDa protein | viruses such as, for example, PLRV, PVY and |
| | ToMoV |
| nuclear inclusion proteins e.g. a or b or | viruses such as, for example, PLRV, PVY and |
| | ToMoV |
| nucleoprotein | TRV |
| pseudoubiquitin | viruses such as, for example, PLRV, PVY and |
| | ToMoV |
| replicase | viruses such as, for example, PLRV, PVY and |
| | ToMoV |
| toxins of Bacillus thuringiensis, VIP 3, | Lepidoptera e.g. Heliothis, whitefly |
| Bacilluscereus toxins, Photorabdus and | aphids |
| Xenorhabdus toxins | |
| 3-hydroxysteroid oxidase | Lepidoptera e.g. Heliothis, whitefly, |
| | aphids |
| peroxidase | Lepidoptera e.g. Heliothis, whitefly, |
| | aphids |
| aminopeptidase inhibitors, e.g. leucine | Lepidoptera e.g. Heliothis, whitefly, |
| aminopeptidase inhibitor | aphids |
| lectins | Lepidoptera e.g. Heliothis, whitefly, |
| | aphids |
| protease inhibitors, e.g. cystatin, patatin | Lepidoptera e.g. Heliothis, whitefly, |
| | aphids |
| ribosome-inactivating protein | Lepidoptera e.g. Heliothis, whitefly, |
| | aphids |
| stilbene synthase | Lepidoptera e.g. Heliothis, whitefly, |
| | aphids |
| HMG-CoA reductase | Lepidoptera e.g. Heliothis, whitefly, |
| | aphids |
| hatching factor for cyst nematodes | cyst nematodes |
| barnase | nematodes, e.g. root-knot nematodes and |
| | cyst nematodes |
| principles for preventing food uptake | nematodes, e.g. root-knot nematodes and |
| | cyst nematodes |

Plant: Bell Pepper

| Structure affected/protein expressed | Feature of the plant/tolerance to |
|---|---|
| acetolactate synthase (ALS) | sulphonylurea compounds, imidazolinones |
| | triazolopyrimidines, |
| | pyrimidyloxybenzoates, phthalides |
| acetyl-CoA carboxylase (ACCase) | aryloxyphenoxyalkanecarboxylic acids, |
| | cyclohexanediones |
| hydroxyphenylpyruvate dioxygenase (HPPD) | isoxazoles such as, for example, isoxaflutole or |
| | isoxachlortole, triones such as, for example, |
| | mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazoles, pyridine derivatives, |
| | phenopylate, oxadiazoles etc. |
| cytochrome P450 e.g. P450 SU1 or selection | xenobiotics and herbicides such as, for |
| | example, sulphonylurea compounds |
| polyphenol oxidase or polyphenol oxidase | bacterial and fungal pathogens |
| (antisense) | |
| metallothionein | bacterial and fungal pathogens |
| ribonuclease | bacterial and fungal pathogens |
| antifungal polypeptid AlyAFP | bacterial and fungal pathogens |
| oxalate oxidase | bacterial and fungal pathogens |
| glucose oxidase | bacterial and fungal pathogens |
| pyrrolnitrin synthesis genes | bacterial and fungal pathogens |
| serine/threonine kinases | bacterial and fungal pathogens |
| cecropin B | bacterial and fungal pathogens, rot, |
| | leaf mould, etc. |
| phenylalanine ammonia lyase (PAL) | bacterial and fungal pathogens |
| Cf genes, e.g. Cf 9 Ct5 Cf4 Cf2 | bacterial and fungal pathogens |
| osmotin | bacterial and fungal pathogens |
| alpha hordothionine | bacterial and fungal pathogens |
| systemin | bacterial and fungal pathogens |
| polygalacturonase inhibitors | bacterial and fungal pathogens |
| Prf control gene | bacterial and fungal pathogens |
| 12 Fusarium resistance site | Fusarium |
| phytoalexins | bacterial and fungal pathogens |
| B-1,3-glucanase (antisense) | bacterial and fungal pathogens |
| receptor kinase | bacterial and fungal pathogens |
| polypeptide having the effect of triggering | bacterial and fungal pathogens |
| a hypersensitivity reaction | |
| systemic aquired resistance (SAR) genes | viral, bacterial, fungal and nematodal |
| | pathogens |
| chitinases | bacterial and fungal pathogens |
| barnase | bacterial and fungal pathogens |
| glucanases | bacterial and fungal pathogens |
| double-strand ribonuclease | viruses such as, for example, CMV, TEV |
| envelope proteins | viruses such as, for example, CMV, TEV |
| 17 kDa or 60 kDa protein | viruses such as, for example, CMV, TEV |
| nuclear inclusion proteins e.g. a or b or | viruses such as, for example, CMV, TEV |
| nucleoprotein | |
| pseudoubiquitin | viruses such as, for example, CMV, TEV |
| replicase | viruses such as, for example, CMV, TEV |
| toxins of Bacillus thuringiensis, VIP 3, | Lepidoptera, whitefly, aphids |
| Bacilluscereus toxins, Photorabdus and | |
| Xenorhabdus toxins | |
| 3-hydroxysteroid oxidase | Lepidoptera, whitefly, aphids |
| peroxidase | Lepidoptera, whitefly, aphids |
| aminopeptidase inhibitors, e.g. leucine | Lepidoptera, whitefly, aphids |
| aminopeptidase inhibitor | |
| lectins | Lepidoptera, whitefly, aphids |
| protease inhibitors, e.g. cystatin, patatin | Lepidoptera, whitefly, aphids |
| ribosome-inactivating protein | Lepidoptera, whitefly, aphids |
| stilbene synthase | Lepidoptera, whitefly, aphids |
| HMG-CoA reductase | Lepidoptera, whitefly, aphids |
| hatching factor for cyst nematodes | cyst nematodes |
| barnase | nematodes, e.g. root-knot nematodes and |
| | cyst nematodes |
| principles for preventing food uptake | nematodes, e.g. root-knot nematodes and |
| | cyst nematodes |

Plant: Grapevines

| Structure affected/principle expressed | Feature of the plant/tolerance to |
|---|---|
| acetolactate synthase (ALS) | sulphonylurea compounds, imidazolinones |
| | triazolopyrimidines, |
| | pyrimidyloxybenzoates, phthalides |
| acetyl-CoA carboxylase (ACCase) | aryloxyphenoxyalkanecarboxylic acids, |
| | cyclohexanediones |
| hydroxyphenylpyruvate dioxygenase (HPPD) | isoxazoles such as, for example, isoxaflutole or |
| | isoxachlortole, triones such as, for example, |
| | mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazoles, pyridine derivatives, |
| | phenopylate, oxadiazoles etc. |
| cytochrome P450 e.g. P450 SU1 or selection | xenobiotics and herbicides such as, for |
| | example, sulphonylurea compounds |
| polyphenol oxidase or polyphenol oxidase | bacterial and fungal pathogens such as |
| (antisense) | Botrytis and powdery mildew |
| metallothionein | bacterial and fungal pathogens such as |
| | Botrytis and powdery mildew |
| ribonuclease | bacterial and fungal pathogens such as |
| | Botrytis and powdery mildew |
| antifungal polypeptide AlyAFP | bacterial and fungal pathogens such as |
| | Botrytis and powdery mildew |
| oxalate oxidase | bacterial and fungal pathogens such as |
| | Botrytis and powdery mildew |
| glucose oxidase | bacterial and fungal pathogens such as |
| | Botrytis and powdery mildew |
| pyrrolnitrin synthesis genes | bacterial and fungal pathogens such as |
| | Botrytis and powdery mildew |
| serine/threonine kinases | bacterial and fungal pathogens such as |
| | Botrytis and powdery mildew |
| cecropin B | bacterial and fungal pathogens such as |
| | Botrytis and powdery mildew |
| phenylalanine ammonia lyase (PAL) | bacterial and fungal pathogens such as |
| | Botrytis and powdery mildew |
| Cf genes, e.g. Cf 9 Cf5 Cf4 Cf2 | bacterial and fungal pathogens such as |
| | Botrytis and powdery mildew |
| osmotin | bacterial and fungal pathogens such as |
| | Botrytis and powdery mildew |
| alpha hordothionine | bacterial and fungal pathogens such as |
| | Botrytis and powdery mildew |
| systemin | bacterial and fungal pathogens such as |
| | Botrytis and powdery mildew |
| polygalacturonase inhibitors | bacterial and fungal pathogens such as |
| | Botrytis and powdery mildew |
| Prf control gene | bacterial and fungal pathogens such as |
| | Botrytis and powdery mildew |
| phytoalexins | bacterial and fungal pathogens such as |
| | Botrytis and powdery mildew |
| B-1,3-glucanase (antisense) | bacterial and fungal pathogens such as |
| | Botrytis and powdery mildew |
| receptor kinase | bacterial and fungal pathogens such as |
| | Botrytis and powdery mildew |
| polypeptide having the effect of triggering | bacterial and fungal pathogens such as Botrytis |
| a hypersensitivity reaction | and powdery mildew |
| systemic aquired resistance (SAR) genes | viral, bacterial, fungal and nematodal |
| | pathogens |
| chitinases | bacterial and fungal pathogens such as |
| | Botrytis and powdery mildew |
| barnase | bacterial and fungal pathogens such as |
| | Botrytis and powdery mildew |
| glucanases | bacterial and fungal pathogens such as Botrytis |
| | and powdery mildew |
| double-strand ribonuclease | viruses |
| envelope proteins | viruses |
| 17 kDa or 60 kDa protein | viruses |
| nuclear inclusion proteins e.g. a or b or | viruses |
| nucleoprotein | |
| pseudoubiquitin | viruses |
| replicase | viruses |
| toxins of Bacillus thuringiensis, VIP 3, | Lepidoptera, aphids |
| Bacilluscereus toxins, Photorabdus and | |
| Xenorhabdus toxins | |
| 3-hydroxysteroid oxidase | Lepidoptera, aphids |
| peroxidase | Lepidoptera, aphids |
| aminopeptidase inhibitors, e.g. leucine | Lepidoptera, aphids |
| aminopeptidase inhibitor | |
| lectins | Lepidoptera, aphids |
| protease inhibitors, e.g. cystatin, patatin | Lepidoptera, aphids |
| ribosome-inactivating protein | Lepidoptera, aphids |
| stilbene synthase | Lepidoptera, aphids, diseases |
| HMG-CoA reductase | Lepidoptera, aphids |
| hatching factor for cyst nematodes | cyst nematodes |
| barnase | nematodes, e.g. root-knot nematodes and |
| | cyst nematodes or general diseases |
| CBI | root-knot nematodes |
| principles for preventing food uptake | nematodes, e.g. root-knot nematodes |
| | or root-cyst nematodes |

Plant: Oilseed rape

| Structure affected/protein expressed | Feature of the plant/tolerance to |
|---|---|
| acetolactate synthase (ALS) | sulphonylurea compounds, imidazolinones |
| | triazolopyrimidines, |
| | pyrimidyloxybenzoates, phthalides |
| acetyl-CoA carboxylase (ACCase) | aryloxyphenoxyalkanecarboxylic acids, |
| | cyclohexanediones |
| hydroxyphenylpyruvate dioxygenase (HPPD) | isoxazoles such as, for example, isoxaflutole or |
| | isoxachlortole, triones such as, for example, |
| | mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazoles, pyridine derivatives, |
| | phenopylate, oxadiazoles etc. |
| cytochrome P450 e.g. P450 SU1 or selection | xenobiotics and herbicides such as, for |
| | example, sulphonylurea compounds |
| polyphenol oxidase or polyphenol | bacterial and fungal pathogens such as |
| oxidase (antisense) | Cylindrosporium, Phoma, Sclerotinia |
| metallothionein | bacterial and fungal pathogens such as |
| | Cylindrosporium, Phoma, Sclerotinia |
| ribonuclease | bacterial and fungal pathogens such as |
| | Cylindrosporium, Phoma, Sclerotinia |
| antifungal polypeptid AlyAFP | bacterial and fungal pathogens such as |
| | Cylindrosporium, Phoma, Sclerotinia |
| oxalate oxidase | bacterial and fungal pathogens such as |
| | Cylindrosporium, Phoma, Sclerotinia |
| glucose oxidase | bacterial and fungal pathogens such as |
| | Cylindrosporium, Phoma, Sclerotinia |
| pyrrolnitrin synthesis genes | bacterial and fungal pathogens such as |
| | Cylindrosporium, Phoma, Sclerotinia |
| serine/threonine kinases | bacterial and fungal pathogens such as |
| | Cylindrosporium, Phoma, Sclerotinia |
| cecropin B | bacterial and fungal pathogens such as |
| | Cylindrosporium, Phoma, Sclerotinia |
| phenylalanine ammonia lyase (PAL) | bacterial and fungal pathogens such as |
| | Cylindrosporium, Phoma, Sclerotinia |
| Cf genes, e.g. Cf 9 Cf5 Cf4 Cf2 | bacterial and fungal pathogens such as |
| | Cylindrosporium, Phoma, Sclerotinia |
| osmotin | bacterial and fungal pathogens such as |
| | Cylindrosporium, Phoma, Sclerotinia |
| alpha hordothionine | bacterial and fungal pathogens such as |
| | Cylindrosporium, Phoma, Sclerotinia |
| systemin | bacterial and fungal pathogens such as |
| | Cylindrosporium, Phoma, Sclerotinia |
| polygalacturonase inhibitors | bacterial and fungal pathogens such as |
| | Cylindrosporium, Phoma, Sclerotinia |
| Prf control gene | bacterial and fungal pathogens such as |
| | Cylindrosporium, Phoma, Sclerotinia |
| phytoalexins | bacterial and fungal pathogens such as |
| | Cylindrosporium, Phoma, Sclerotinia |
| B-1,3-glucanase (antisense) | bacterial and fungal pathogens such as |
| | Cylindrosporium, Phoma, Sclerotinia |
| receptor kinase | bacterial and fungal pathogens such as |
| | Cylindrosporium, Phoma, Sclerotinia |
| polypeptide having the effect of triggering | bacterial and fungal pathogens such as |
| a hypersensitivity reaction | Cylindrosporium, Phoma, Sclerotinia |
| systemic aquired resistance (SAR) genes | viral, bacterial, fungal and nematodal |
| | pathogens |
| chitinases | bacterial and fungal pathogens such as |
| | Cylindrosporium, Phoma, Sclerotinia |
| barnase | bacterial and fungal pathogens such as |
| | Cylindrosporium, Phoma, Sclerotinia, |
| | nematodes |
| glucanases | bacterial and fungal pathogens such as |
| | Cylindrosporium, Phoma, Sclerotinia |
| double-strand ribonuclease | viruses |
| envelope proteins | viruses |
| 17 kDa or 60 kDa protein | viruses |
| nuclear inclusion proteins e.g. a or b or | viruses |
| nucleoprotein | |
| pseudoubiquitin | viruses |
| replicase | viruses |
| toxins of Bacillus thuringiensis, VIP 3, | Lepidoptera, aphids |
| Bacilluscereus toxins, Photorabdus and | |
| Xenorhabdus toxins | |
| 3-hydroxysteroid oxidase | Lepidoptera, aphids |
| peroxidase | Lepidoptera, aphids |
| aminopeptidase inhibitors, e.g. leucine | Lepidoptera, aphids |
| aminopeptidase inhibitor | |
| lectins | Lepidoptera, aphids |
| protease inhibitors, e.g. cystatin, patatin, | Lepidoptera, aphids |
| CPTI | |
| ribosome-inactivating protein | Lepidoptera, aphids |
| stilbene synthase | Lepidoptera, aphids, diseases |
| HMG-CoA reductase | Lepidoptera, aphids |
| hatching factor for cyst nematodes | cyst nematodes |
| barnase | nematodes, e.g. root-knot nematodes and |
| | cyst nematodes |
| CBI | root-knot nematodes |
| principles for preventing food uptake | nematodes, e.g. root-knot nematodes and |
| induced at nematode feeding sites | root-cyst nematodes |

Plant: Brassica vegetables (cabbage, Brussels sprouts etc.)

| Structure affected/protein expressed | Feature of the plant/tolerance to |
|---|---|
| acetolactate synthase (ALS) | sulphonylurea compounds, imidazolinones |
| | triazolopyrimidines, |
| | pyrimidyloxybenzoates, phthalides |
| acetyl-CoA carboxylase (ACCase) | aryloxyphenoxyalkanecarboxylic acids, |
| | cyclohexanediones |
| hydroxyphenylpyruvate dioxygenase (HPPD) | isoxazoles such as, for example, isoxaflutole or |
| | isoxachlortole, triones such as, for example, |
| | mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazoles, pyridine derivatives, |
| | phenopylate, oxadiazoles etc. |
| cytochrome P450 e.g. P450 SU1 or selection | xenobiotics and herbicides such as, for |
| | example, sulphonylurea compounds |
| polyphenol oxidase or polyphenol oxidase | bacterial and fungal pathogens |
| (antisense) | |
| metallothionein | bacterial and fungal pathogens |
| ribonuclease | bacterial and fungal pathogens |
| antifungal polypeptid AlyAFP | bacterial and fungal pathogens |
| oxalate oxidase | bacterial and fungal pathogens |
| glucose oxidase | bacterial and fungal pathogens |
| pyrrolnitrin synthesis genes | bacterial and fungal pathogens |
| serine/threonine kinases | bacterial and fungal pathogens |
| cecropin B | bacterial and fungal pathogens |
| phenylalanine ammonia lyase (PAL) | bacterial and fungal pathogens |
| Cf genes, e.g. Cf 9 Cf5 Cf4 Cf2 | bacterial and fungal pathogens |
| osmotin | bacterial and fungal pathogens |
| alpha hordothionine | bacterial and fungal pathogens |
| systemin | bacterial and fungal pathogens |
| polygalacturonase inhibitors | bacterial and fungal pathogens |
| Prf control gene | bacterial and fungal pathogens |
| phytoalexins | bacterial and fungal pathogens |
| B-1,3-glucanase (antisense) | bacterial and fungal pathogens |
| receptor kinase | bacterial and fungal pathogens |
| polypeptide having the effect of triggering | bacterial and fungal pathogens |
| a hypersensitivity reaction | |
| systemic aquired resistance (SAR) genes | viral, bacterial, fungal and nematodal |
| | pathogens |
| chitinases | bacterial and fungal pathogens |
| barnase | bacterial and fungal pathogens |
| glucanases | bacterial and fungal pathogens |
| double-strand ribonuclease | viruses |
| envelope proteins | viruses |
| 17 kDa or 60 kDa protein | viruses |
| nuclear inclusion proteins e.g. a or b or | viruses |
| nucleoprotein | |
| pseudoubiquitin | viruses |
| replicase | viruses |
| toxins of Bacillus thuringiensis, VIP 3, | Lepidoptera, aphids |
| Bacilluscereus toxins, Photorabdus and | |
| Xenorhabdus toxins | |
| 3-hydroxysteroid oxidase | Lepidoptera, aphids |
| peroxidase | Lepidoptera, aphids |
| aminopeptidase inhibitors, e.g. leucine | Lepidoptera, aphids |
| aminopeptidase inhibitor | |
| lectins | Lepidoptera, aphids |
| protease inhibitors, e.g. cystatin, patatin, | Lepidoptera, aphids |
| CPTI | |
| ribosome-inactivating protein | Lepidoptera, aphids |
| stilbene synthase | Lepidoptera, aphids, diseases |
| HMG-CoA reductase | Lepidoptera, aphids |
| hatching factor for cyst nematodes | cyst nematodes |
| barnase | nematodes, e.g. root-knot nematodes and |
| | cyst nematodes |
| CBI | root-knot nematodes |
| principles for preventing food uptake | nematodes, e.g. root-knot nematodes and |
| induced at nematode feeding sites | root-cyst nematodes |
| | cyst nematodes |

Plants: Pomaceous fruit, e.g. apples, pears

| Structure affected/protein expressed | Feature of the plant/tolerance to |
|---|---|
| acetolactate synthase (ALS) | sulphonylurea compounds, imidazolinones |
| | triazolopyrimidines, |
| | pyrimidyloxybenzoates, phthalides |
| acetyl-CoA carboxylase (ACCase) | aryloxyphenoxyalkanecarboxylic acids, |
| | cyclohexanediones |
| hydroxyphenylpyruvate dioxygenase (HPPD) | isoxazoles such as, for example, isoxaflutole or |
| | isoxachlortole, triones such as, for example, |
| | mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazoles, pyridine derivatives, |
| | phenopylate, oxadiazoles etc. |
| cytochrome P450 e.g. P450 SU1 or selection | xenobiotics and herbicides such as, for |
| | example, sulphonylurea compounds |
| polyphenol oxidase or polyphenol oxidase | bacterial and fungal pathogens such as |
| (antisense) | storage scab on apples or fire-blight |
| metallothionein | bacterial and fungal pathogens such as |
| | storage scab on apples or fire-blight |
| ribonuclease | bacterial and fungal pathogens such as |
| | storage scab on apples or fire-blight |
| antifungal polypeptid AlyAFP | bacterial and fungal pathogens such as |
| | storage scab on apples or fire-blight |
| oxalate oxidase | bacterial and fungal pathogens such as |
| | storage scab on apples or fire-blight |
| glucose oxidase | bacterial and fungal pathogens such as |
| | storage scab on apples or fire-blight |
| pyrrolnitrin synthesis genes | bacterial and fungal pathogens such as |
| | storage scab on apples or fire-blight |
| serine/threonine kinases | bacterial and fungal pathogens such as |
| | storage scab on apples or fire-blight |
| cecropin B | bacterial and fungal pathogens such as |
| | storage scab on apples or fire-blight |
| phenylalanine ammonia lyase (PAL) | bacterial and fungal pathogens such as |
| | storage scab on apples or fire-blight |
| Cf genes, e.g. Cf 9 Cf5 Cf4 Cf2 | bacterial and fungal pathogens such as |
| | storage scab on apples or fire-blight |
| osmotin | bacterial and fungal pathogens such as |
| | storage scab on apples or fire-blight |
| alpha hordothionine | bacterial and fungal pathogens such as |
| | storage scab on apples or fire-blight |
| systemin | bacterial and fungal pathogens such as |
| | storage scab on apples or fire-blight |
| polygalacturonase inhibitors | bacterial and fungal pathogens such as |
| | storage scab on apples or fire-blight |
| Prf control gene | bacterial and fungal pathogens such as |
| | storage scab on apples or fire-blight |
| phytoalexins | bacterial and fungal pathogens such as |
| | storage scab on apples or fire-blight |
| B-1,3-glucanase (antisense) | bacterial and fungal pathogens such as |
| | storage scab on apples or fire-blight |
| receptor kinase | bacterial and fungal pathogens such as |
| | storage scab on apples or fire-blight |
| polypeptide having the effect of triggering | bacterial and fungal pathogens such as |
| a hypersensitivity reaction | storage scab on apples or fire-blight |
| systemic aquired resistance (SAR) genes | viral, bacterial, fungal and nematodal |
| | pathogens |
| lytic protein | bacterial and fungal pathogens such as |
| | storage scab on apples or fire-blight |
| lysozyme | bacterial and fungal pathogens such as |
| | storage scab on apples or fire-blight |
| chitinases | bacterial and fungal pathogens such as |
| | storage scab on apples or fire-blight |
| barnase | bacterial and fungal pathogens such as |
| | storage scab on apples or fire-blight |
| glucanases | bacterial and fungal pathogens such as |
| | storage scab on apples or fire-blight |
| double-strand ribonuclease | viruses |
| envelope proteins | viruses |
| 17 kDa or 60 kDa protein | viruses |
| nuclear inclusion proteins e.g. a or b or | viruses |
| nucleoprotein | |
| pseudoubiquitin | viruses |
| replicase | viruses |
| toxins of Bacillus thuringiensis, VIP 3, | Lepidoptera, aphids, mites |
| Bacilluscereus toxins, Photorabdus and | |
| Xenorhabdus toxins | |
| 3-hydroxysteroid oxidase | Lepidoptera, aphids, mites |
| peroxidase | Lepidoptera, aphids, mites |
| aminopeptidase inhibitors, e.g. leucine | Lepidoptera, aphids, mites |
| aminopeptidase inhibitor | |
| lectins | Lepidoptera, aphids, mites |
| protease inhibitors, e.g. cystatin, patatin, | Lepidoptera, aphids, mites |
| CPTI | |
| ribosome-inactivating protein | Lepidoptera, aphids, mites |
| stilbene synthase | Lepidoptera, aphids, diseases, mites |
| HMG-CoA reductase | Lepidoptera, aphids, mites |
| hatching factor for cyst nematodes | cyst nematodes |
| barnase | nematodes, e.g. root-knot nematodes and |
| | cyst nematodes |
| CBI | root-knot nematodes |
| principles for preventing food uptake | nematodes, e.g. root-knot nematodes and |
| induced at nematode feeding sites | root-cyst nematodes |

Plant: Melon

| Structure affected/protein expressed | Feature of the plant/tolerance to |
|---|---|
| acetolactate synthase (ALS) | sulphonylurea compounds, imidazolinones |
| | triazolopyrimidines, |
| | pyrimidyloxybenzoates, phthalides |
| acetyl-CoA carboxylase (ACCase) | aryloxyphenoxyalkanecarboxylic acids, |
| | cyclohexanediones |
| hydroxyphenylpyruvate dioxygenase (HPPD) | isoxazoles such as, for example, isoxaflutole or |
| | isoxachlortole, triones such as, for example, |
| | mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazoles, pyridine derivatives, |
| | phenopylate, oxadiazoles etc. |
| cytochrome P450 e.g. P450 SU1 or selection | xenobiotics and herbicides such as, for |
| | example, sulphonylurea compounds |
| polyphenol oxidase or polyphenol oxidase | bacterial or fungal pathogens such as |
| (antisense) | Phytophtora |
| metallothionein | bacterial or fungal pathogens such as |
| | Phytophtora |
| ribonuclease | bacterial or fungal pathogens such as |
| | Phytophtora |
| antifungal polypeptid AlyAFP | bacterial or fungal pathogens such as |
| | Phytophtora |
| oxalate oxidase | bacterial or fungal pathogens such as |
| | Phytophtora |
| glucose oxidase | bacterial or fungal pathogens such as |
| | Phytophtora |
| pyrrolnitrin synthesis genes | bacterial or fungal pathogens such as |
| | Phytophtora |
| serine/threonine kinases | bacterial or fungal pathogens such as |
| | Phytophtora |
| cecropin B | bacterial or fungal pathogens such as |
| | Phytophtora |
| phenylalanine ammonia lyase (PAL) | bacterial or fungal pathogens such as |
| | Phytophtora |
| Cf genes, e.g. Cf 9 Cf5 Cf4 Cf2 | bacterial or fungal pathogens such as |
| | Phytophtora |
| osmotin | bacterial or fungal pathogens such as |
| | Phytophtora |
| alpha hordothionine | bacterial or fungal pathogens such as |
| | Phytophtora |
| systemin | bacterial or fungal pathogens such as |
| | Phytophtora |
| polygalacturonase inhibitors | bacterial or fungal pathogens such as |
| | Phytophtora |
| Prf control gene | bacterial or fungal pathogens such as |
| | Phytophtora |
| phytoalexins | bacterial or fungal pathogens such as |
| | Phytophtora |
| B-1,3-glucanase (antisense) | bacterial or fungal pathogens such as |
| | Phytophtora |
| receptor kinase | bacterial or fungal pathogens such as |
| | Phytophtora |
| polypeptide having the effect of triggering | bacterial or fungal pathogens such as |
| a hypersensitivity reaction | Phytophtora |
| systemic aquired resistance (SAR) genes | viral, bacterial, fungal and nematodal |
| | pathogens |
| lytic protein | bacterial or fungal pathogens such as |
| | Phytophtora |
| lysozyme | bacterial or fungal pathogens such as |
| | Phytophtora |
| chitinases | bacterial or fungal pathogens such as |
| | Phytophtora |
| barnase | bacterial or fungal pathogens such as |
| | Phytophtora |
| glucanases | bacterial or fungal pathogens such as |
| | Phytophtora |
| double-strand ribonuclease | viruses such as CMV, PRSV, WMV2, SMV, |
| | ZYMV |
| envelope proteins | viruses such as CMV, PRSV, WMV2, SMV, |
| | ZYMV |
| 17 kDa or 60 kDa protein | viruses such as CMV, PRSV, WMV2, SMV, |
| | ZYMV |
| nuclear inclusion proteins e.g. a or b or | viruses such as CMV, PRSV, WMV2, SMV, |
| nucleoprotein | ZYMV |
| pseudoubiquitin | viruses such as CMV, PRSV, WMV2, SMV, |
| | ZYMV |
| replicase | viruses such as CMV, PRSV, WMV2, SMV, |
| | ZYMV |
| toxins of Bacillus thuringiensis, VIP 3, | Lepidoptera, aphids, mites |
| Bacilluscereus toxins, Photorabdus and | |
| Xenorhabdus toxins | |
| 3-hydroxysteroid oxidase | Lepidoptera, aphids, mites, whitefly |
| peroxidase | Lepidoptera, aphids, mites, whitefly |
| aminopeptidase inhibitors, e.g. leucine | Lepidoptera, aphids, mites, whitefly |
| aminopeptidase inhibitor | |
| lectins | Lepidoptera, aphids, mites, whitefly |
| protease inhibitors, e.g. cystatin, patatin, | Lepidoptera, aphids, mites, whitefly |
| CPTI, virgiferin | |
| ribosome-inactivating protein | Lepidoptera, aphids, mites, whitefly |
| stilbene synthase | Lepidoptera, aphids, mites, whitefly |
| HMG-CoA reductase | Lepidoptera, aphids, mites, whitefly |
| hatching factor for cyst nematodes | cyst nematodes |
| barnase | nematodes, e.g. root-knot nematodes and |
| | cyst nematodes |
| CBI | root-knot nematodes |
| principles for preventing food uptake | nematodes, e.g. root-knot nematodes and |
| induced at nematode feeding sites | root-cyst nematodes |

Plant: Banana

| Structure affected/protein expressed | Feature of the plant/tolerance to |
|---|---|
| acetolactate synthase (ALS) | sulphonylurea compounds, imidazolinones |
| | triazolopyrimidines, |
| | pyrimidyloxybenzoates, phthalides |
| acetyl-CoA carboxylase (ACCase) | aryloxyphenoxyalkanecarboxylic acids, |
| | cyclohexanediones |
| hydroxyphenylpyruvate dioxygenase (HPPD) | isoxazoles such as, for example, isoxaflutole or |
| | isoxachlortole, triones such as, for example, |
| | mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazoles, pyridine derivatives, |
| | phenopylate, oxadiazoles etc. |
| cytochrome P450 e.g. P450 SU1 or selection | xenobiotics and herbicides such as, for |
| | example, sulphonylurea compounds |
| polyphenol oxidase or polyphenol oxidase | bacterial or fungal pathogens |
| (antisense) | |
| metallothionein | bacterial or fungal pathogens |
| ribonuclease | bacterial or fungal pathogens |
| antifungal polypeptid AlyAFP | bacterial or fungal pathogens |
| oxalate oxidase | bacterial or fungal pathogens |
| glucose oxidase | bacterial or fungal pathogens |
| pyrrolnitrin synthesis genes | bacterial or fungal pathogens |
| serine/threonine kinases | bacterial or fungal pathogens |
| cecropin B | bacterial or fungal pathogens |
| phenylalanine ammonia lyase (PAL) | bacterial or fungal pathogens |
| Cf genes, e.g. Cf 9 Cf5 Cf4 Cf2 | bacterial or fungal pathogens |
| osmotin | bacterial or fungal pathogens |
| alpha hordothionine | bacterial or fungal pathogens |
| systemin | bacterial or fungal pathogens |
| polygalacturonase inhibitors | bacterial or fungal pathogens |
| Prf control gene | bacterial or fungal pathogens |
| phytoalexins | bacterial or fungal pathogens |
| B-1,3-glucanase (antisense) | bacterial or fungal pathogens |
| receptor kinase | bacterial or fungal pathogens |
| polypeptide having the effect of triggering | bacterial or fungal pathogens |
| a hypersensitivity reaction | |
| systemic aquired resistance (SAR) genes | viral, bacterial, fungal and nematodal |
| | pathogens |
| lytic protein | bacterial or fungal pathogens |
| lysozyme | bacterial or fungal pathogens |
| chitinases | bacterial or fungal pathogens |
| barnase | bacterial or fungal pathogens |
| glucanases | bacterial or fungal pathogens |
| double-strand ribonuclease | viruses such as the Banana Bunchy Top Virus |
| | (BBTV) |
| envelope proteins | viruses such as the Banana Bunchy Top Virus |
| | (BBTV) |
| 17 kDa or 60 kDa protein | viruses such as the Banana Bunchy Top Virus |
| | (BBTV) |
| nuclear inclusion proteins e.g. a or b or | viruses such as the Banana Bunchy Top Virus |
| nucleoprotein | (BBTV) |
| pseudoubiquitin | viruses such as the Banana Bunchy Top Virus |
| | (BBTV) |
| replicase | viruses such as the Banana Bunchy Top Virus |
| | (BBTV) |
| toxins of Bacillus thuringiensis, VIP 3, | Lepidoptera, aphids, mites, nematodes |
| Bacilluscereus toxins, Photorabdus and | |
| Xenorhabdus toxins | |
| 3-hydroxysteroid oxidase | Lepidoptera, aphids, mites, nematodes |
| peroxidase | Lepidoptera, aphids, mites, nematodes |
| aminopeptidase inhibitors, e.g. leucine | Lepidoptera, aphids, mites, nematodes |
| aminopeptidase inhibitor | |
| lectins | Lepidoptera, aphids, mites, nematodes |
| protease inhibitors, e.g. cystatin, patatin, | Lepidoptera, aphids, mites, nematodes |
| CPTI, virgiferin | |
| ribosome-inactivating protein | Lepidoptera, aphids, mites, nematodes |
| stilbene synthase | Lepidoptera, aphids, mites, nematodes |
| HMG-CoA reductase | Lepidoptera, aphids, mites, nematodes |
| hatching factor for cyst nematodes | cyst nematodes |
| barnase | nematodes, e.g. root-knot nematodes and |
| | cyst nematodes |
| CBI | root-knot nematodes |
| principles for preventing food uptake | nematodes, e.g. root-knot nematodes and |
| induced at nematode feeding sites | root-cyst nematodes |

Plant: Cotton

| Structure affected/protein expressed | Feature of the plant/tolerance to |
|---|---|
| acetolactate synthase (ALS) | sulphonylurea compounds, imidazolinones |
| | triazolopyrimidines, |
| | pyrimidyloxybenzoates, phthalides |
| acetyl-CoA carboxylase (ACCase) | aryloxyphenoxyalkanecarboxylic acids, |
| | cyclohexanediones |
| hydroxyphenylpyruvate dioxygenase (HPPD) | isoxazoles such as, for example, isoxaflutole or |
| | isoxachlortole, triones such as, for example, |
| | mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthese |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazoles, pyridine derivatives, |
| | phenopylate, oxadiazoles etc. |
| cytochrome P450 e.g. P450 SU1 or selection | xenobiotics and herbicides such as, for |
| | example, sulphonylurea compounds |
| polyphenol oxidase or polyphenol oxidase | bacterial or fungal pathogens |
| (antisense) | |
| metallothionein | bacterial or fungal pathogens |
| ribonuclease | bacterial or fungal pathogens |
| antifungal polypeptid AlyAFP | bacterial or fungal pathogens |
| oxalate oxidase | bacterial or fungal pathogens |
| glucose oxidase | bacterial or fungal pathogens |
| pyrrolnitrin synthesis genes | bacterial or fungal pathogens |
| serine/threonine kinases | bacterial or fungal pathogens |
| cecropin B | bacterial or fungal pathogens |
| phenylalanine ammonia lyase (PAL) | bacterial or fungal pathogens |
| Cf genes, e.g. Cf 9 Cf5 Cf4 Cf2 | bacterial or fungal pathogens |
| osmotin | bacterial or fungal pathogens |
| alpha hordothionine | bacterial or fungal pathogens |
| systemin | bacterial or fungal pathogens |
| polygalacturonase inhibitors | bacterial or fungal pathogens |
| Prf control gene | bacterial or fungal pathogens |
| phytoalexins | bacterial or fungal pathogens |
| B-1,3-glucanase (antisense) | bacterial or fungal pathogens |
| receptor kinase | bacterial or fungal pathogens |
| polypeptide having the effect of triggering | bacterial or fungal pathogens |
| a hypersensitivity reaction | |
| systemic aquired resistance (SAR) genes | viral, bacterial, fungal and nematodal |
| | pathogens |
| lytic protein | bacterial or fungal pathogens |
| lysozyme | bacterial or fungal pathogens |
| chitinases | bacterial or fungal pathogens |
| barnase | bacterial or fungal pathogens |
| glucanases | bacterial or fungal pathogens |
| double-strand ribonuclease | viruses such as the wound tumour virus (WTV) |
| envelope proteins | viruses such as the wound tumour virus (WTV) |
| 17 kDa or 60 kDa protein | viruses such as the wound tumour virus (WTV) |
| nuclear inclusion proteins e.g. a or b or | viruses such as the wound tumour virus (WTV) |
| nucleoprotein | |
| pseudoubiquitin | viruses such as the wound tumour virus (WTV) |
| replicase | viruses such as the wound tumour virus (WTV) |
| toxins of Bacillus thuringiensis, VIP 3, | Lepidoptera, aphids, mites, nematodes, |
| Bacilluscereus toxins, Photorabdus and | whitefly |
| Xenorhabdus toxins | |
| 3-hydroxysteroid oxidase | Lepidoptera, aphids, mites, nematodes, |
| | whitefly |
| peroxidase | Lepidoptera, aphids, mites, nematodes, |
| | whitefly |
| aminopeptidase inhibitors, e.g. leucine | Lepidoptera, aphids, mites, nematodes, |
| aminopeptidase inhibitor | whitefly |
| lectins | Lepidoptera, aphids, mites, nematodes, |
| | whitefly |
| protease inhibitors, e.g. cystatin, patatin, | Lepidoptera, aphids, mites, nematodes, |
| CPTI, virgiferin | whitefly |
| ribosome-inactivating protein | Lepidoptera, aphids, mites, nematodes, |
| | whitefly |
| stilbene synthase | Lepidoptera, aphids, mites, nematodes, |
| | whitefly |
| HMG-CoA reductase | Lepidoptera, aphids, mites, nematodes, |
| | whitefly |
| hatching factor for cyst nematodes | cyst nematodes |
| barnase | nematodes, e.g. root-knot nematodes and |
| | cyst nematodes |
| CBI | root-knot nematodes |
| principles for preventing food uptake | nematodes, e.g. root-knot nematodes and |
| induced at nematode feeding sites | root-cyst nematodes |

Plant: Sugar cane

| Feature affected/protein expressed | Feature of the plant/tolerance to |
|---|---|
| acetolactate synthase (ALS) | sulphonylurea compounds, imidazolinones |
| | triazolopyrimidines, |
| | pyrimidyloxybenzoates, phthalides |
| acetyl-CoA carboxylase (ACCase) | aryloxyphenoxyalkanecarboxylic acids, |
| | cyclohexanediones |
| hydroxyphenylpyruvate dioxygenase (HPPD) | isoxazoles such as, for example, isoxaflutole or |
| | isoxachlortole, triones such as, for example, |
| | mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazoles, pyridine derivatives, |
| | phenopylate, oxadiazoles etc. |
| cytochrome P450 e.g. P450 SU1 or selection | xenobiotics and herbicides such as, for |
| | example, sulphonylurea compounds |
| polyphenol oxidase or polyphenol oxidase | bacterial or fungal pathogens |
| (antisense) | |
| metallothionein | bacterial or fungal pathogens |
| ribonuclease | bacterial or fungal pathogens |
| antifungal polypeptid AlyAFP | bacterial or fungal pathogens |
| oxalate oxidase | bacterial or fungal pathogens |
| glucose oxidase | bacterial or fungal pathogens |
| pyrrolnitrin synthesis genes | bacterial or fungal pathogens |
| serine/threonine kinases | bacterial or fungal pathogens |
| cecropin B | bacterial or fungal pathogens |
| phenylalanine ammonia lyase (PAL) | bacterial or fungal pathogens |
| Cf genes, e.g. Cf 9 Cf5 Cf4 Cf2 | bacterial or fungal pathogens |
| osmotin | bacterial or fungal pathogens |
| alpha hordothionine | bacterial or fungal pathogens |
| systemin | bacterial or fungal pathogens |
| polygalacturonase inhibitors | bacterial or fungal pathogens |
| Prf control gene | bacterial or fungal pathogens |
| phytoalexins | bacterial or fungal pathogens |
| B-1,3-glucanase (antisense) | bacterial or fungal pathogens |
| receptor kinase | bacterial or fungal pathogens |
| polypeptide having the effect of triggering | bacterial or fungal pathogens |
| a hypersensitivity reaction | |
| systemic aquired resistance (SAR) genes | viral, bacterial, fungal and nematodal |
| | pathogens |
| lytic protein | bacterial or fungal pathogens |
| lysozyme | bacterial or fungal pathogens, e.g. |
| | Clavibacter |
| chitinases | bacterial or fungal pathogens |
| barnase | bacterial or fungal pathogens |
| glucanases | bacterial or fungal pathogens |
| double-strand ribonuclease | viruses such as SCMV, SrMV |
| envelope proteins | viruses such as SCMV, SrMV |
| 17 kDa or 60 kDa protein | viruses such as SCMV, SrMV |
| nuclear inclusion proteins e.g. a or b or | viruses such as SCMV, SrMV |
| nucleoprotein | |
| pseudoubiquitin | viruses such as SCMV, SrMV |
| replicase | viruses such as SCMV, SrMV |
| toxins of Bacillus thuringiensis, VIP 3, | Lepidoptera, aphids, mites, nematodes, |
| Bacilluscereus toxins, Photorabdus and | whitefly, beetles such as e.g. the Mexican |
| Xenorhabdus toxins | rice borer |
| 3-hydroxysteroid oxidase | Lepidoptera, aphids, mites, nematodes, |
| | whitefly, beetles such as e.g. the Mexican |
| | rice borer |
| peroxidase | Lepidoptera, aphids, mites, nematodes, |
| | whitefly, beetles such as e.g. the Mexican |
| | rice borer |
| aminopeptidase inhibitors, e.g. leucine | Lepidoptera, aphids, mites, nematodes, |
| aminopeptidase inhibitor | whitefly, beetles such as e.g. the Mexican |
| | rice borer |
| lectins | Lepidoptera, aphids, mites, nematodes, |
| | whitefly, beetles such as e.g. the Mexican |
| | rice borer |
| protease inhibitors, e.g. cystatin, patatin, | Lepidoptera, aphids, mites, nematodes, |
| CPTI, virgiferin | whitefly, beetles such as e.g. the Mexican |
| | rice borer |
| ribosome-inactivating protein | Lepidoptera, aphids, mites, nematodes, |
| | whitefly, beetles such as e.g. the Mexican |
| | rice borer |
| stilbene synthase | Lepidoptera, aphids, mites, nematodes, |
| | whitefly, beetles such as e.g. the Mexican |
| | rice borer |
| HMG-CoA reductase | Lepidoptera, aphids, mites, nematodes, |
| | whitefly, beetles such as e.g. the Mexican |
| | rice borer |
| hatching factor for cyst nematodes | cyst nematodes |
| barnase | nematodes, e.g. root-knot nematodes and |
| | cyst nematodes |
| CBI | root-knot nematodes |
| principles for preventing food uptake | nematodes, e.g. root-knot nematodes and |
| induced at nematode feeding sites | root-cyst nematodes |

Plant: Sunflower

| Structure affected/protein expressed | Feature of the plant/tolerance to |
|---|---|
| acetolactate synthase (ALS) | sulphonylurea compounds, imidazolinones |
| | triazolopyrimidines, |
| | pyrimidyloxybenzoates, phthalides |
| acetyl-CoA carboxylase (ACCase) | aryloxyphenoxyalkanecarboxylic acids, |
| | cyclohexanediones |
| hydroxyphenylpyruvate dioxygenase (HPPD) | isoxazoles such as, for example, isoxaflutole or |
| | isoxachlortole, triones such as, for example, |
| | mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazoles, pyridine derivatives, |
| | phenopylate, oxadiazoles etc. |
| cytochrome P450 e.g. P450 SU1 or selection | xenobiotics and herbicides such as, for |
| | example, |
| | sulphonylurea compounds |
| polyphenol oxidase or polyphenol oxidase | bacterial or fungal pathogens |
| (antisense) | |
| metallothionein | bacterial or fungal pathogens |
| ribonuclease | bacterial or fungal pathogens |
| antifungal polypeptid AlyAFP | bacterial or fungal pathogens |
| oxalate oxidase | bacterial or fungal pathogens, e.g. |
| | Sclerotinia |
| glucose oxidase | bacterial or fungal pathogens |
| pyrrolnitrin synthesis genes | bacterial or fungal pathogens |
| serine/threonine kinases | bacterial or fungal pathogens |
| cecropin B | bacterial or fungal pathogens |
| phenylalanine ammonia lyase (PAL) | bacterial or fungal pathogens |
| Cf genes, e.g. Cf 9 Cf5 Cf4 Cf2 | bacterial or fungal pathogens |
| osmotin | bacterial or fungal pathogens |
| alpha hordothionine | bacterial or fungal pathogens |
| systemin | bacterial or fungal pathogens |
| polygalacturonase inhibitors | bacterial or fungal pathogens |
| Prf control gene | bacterial or fungal pathogens |
| phytoalexins | bacterial or fungal pathogens |
| B-1,3-glucanase (antisense) | bacterial or fungal pathogens |
| receptor kinase | bacterial or fungal pathogens |
| polypeptide having the effect of triggering | bacterial or fungal pathogens |
| a hypersensitivity reaction | |
| systemic aquired resistance (SAR) genes | viral, bacterial, fungal and nematodal |
| | pathogens |
| lytic protein | bacterial or fungal pathogens |
| lysozyme | bacterial or fungal pathogens |
| chitinases | bacterial or fungal pathogens |
| barnase | bacterial or fungal pathogens |
| glucanases | bacterial or fungal pathogens |
| double-strand ribonuclease | viruses such as CMV, TMV |
| envelope proteins | viruses such as CMV, TMV |
| 17 kDa or 60 kDa protein | viruses such as CMV, TMV |
| nuclear inclusion proteins e.g. a or b or | viruses such as CMV, TMV |
| nucleoprotein | |
| pseudoubiquitin | viruses such as CMV, TMV |
| replicase | viruses such as CMV, TMV |
| toxins of Bacillus thuringiensis, VIP 3, | Lepidoptera, aphids, mites, nematodes, |
| Bacilluscereus toxins, Photorabdus and | whitefly, beetles |
| Xenorhabdus toxins | |
| 3-hydroxysteroid oxidase | Lepidoptera, aphids, mites, nematodes, |
| | whitefly, beetles |
| peroxidase | Lepidoptera, aphids, mites, nematodes, |
| | whitefly, beetles |
| aminopeptidase inhibitors, e.g. leucine | Lepidoptera, aphids, mites, nematodes, |
| aminopeptidase inhibitor | whitefly, beetles |
| lectins | Lepidoptera, aphids, mites, nematodes, |
| | whitefly, beetles |
| protease inhibitors, e.g. cystatin, patatin, | Lepidoptera, aphids, mites, nematodes, |
| CPTI, virgiferin | whitefly, beetles |
| ribosome-inactivating protein | Lepidoptera, aphids, mites, nematodes, |
| | whitefly, beetles |
| stilbene synthase | Lepidoptera, aphids, mites, nematodes, |
| | whitefly, beetles |
| HMG-CoA reductase | Lepidoptera, aphids, mites, nematodes, |
| | whitefly, beetles |
| hatching factor for cyst nematodes | cyst nematodes |
| barnase | nematodes, e.g. root-knot nematodes and |
| | cyst nematodes |
| CBI | root-knot nematodes |
| principles for preventing food uptake | nematodes, e.g. root-knot nematodes and |
| induced at nematode feeding sites | root-cyst nematodes |

Plants: Sugar beet, turnips

| Structure affected/protein expressed | Feature of the plant/tolerance to |
|---|---|
| acetolactate synthase (ALS) | sulphonylurea compounds, imidazolinones |
| | triazolopyrimidines, |
| | pyrimidyloxybenzoates, phthalides |
| acetyl-CoA carboxylase (ACCase) | aryloxyphenoxyalkanecarboxylic acids, |
| | cyclohexanediones |
| hydroxyphenylpyruvate dioxygenase (HPPD) | isoxazoles such as, for example, isoxaflutole or |
| | isoxachlortole, triones such as, for example, |
| | mesotrione or sulcotrione |
| phosphinothricin acetyltransferase | phosphinothricin |
| O-methyl transferase | modified lignin content |
| glutamine synthetase | glufosinate, bialaphos |
| adenylosuccinate lyase (ADSL) | inhibitors of IMP and AMP synthesis |
| adenylosuccinate synthase | inhibitors of adenylosuccinate synthesis |
| anthranilate synthase | inhibitors of tryptophan synthesis and |
| | degradation |
| nitrilase | 3,5-dihalo-4-hydroxybenzonitriles such as |
| | bromoxynil and loxinyl |
| 5-enolpyruvyl-3-phosphoshikimate | glyphosate or sulphosate |
| synthase (EPSPS) | |
| glyphosate oxidoreductase | glyphosate or sulphosate |
| protoporphyrinogen oxidase (PROTOX) | diphenyl ethers, cyclic imides, |
| | phenylpyrazoles, pyridine derivatives, |
| | phenopylate, oxadiazoles etc. |
| cytochrome P450 e.g. P450 SU1 or selection | xenobiotics and herbicides such as, for |
| | example, sulphonylurea compounds |
| polyphenol oxidase or polyphenol oxidase | bacterial or fungal pathogens |
| (antisense) | |
| metallothionein | bacterial or fungal pathogens |
| ribonuclease | bacterial or fungal pathogens |
| antifungal polypeptid AlyAFP | bacterial or fungal pathogens |
| oxalate oxidase | bacterial or fungal pathogens, e.g. |
| | Sclerotinia |
| glucose oxidase | bacterial or fungal pathogens |
| pyrrolnitrin synthesis genes | bacterial or fungal pathogens |
| serine/threonine kinases | bacterial or fungal pathogens |
| cecropin B | bacterial or fungal pathogens |
| phenylalanine ammonia lyase (PAL) | bacterial or fungal pathogens |
| Cf genes, e.g. Cf 9 Cf5 Cf4 Cf2 | bacterial or fungal pathogens |
| osmotin | bacterial or fungal pathogens |
| alpha hordothionine | bacterial or fungal pathogens |
| systemin | Bakterielle or Pilz_pathogens |
| polygalacturonase inhibitors | bacterial or fungal pathogens |
| Prf control gene | bacterial or fungal pathogens |
| phytoalexins | bacterial or fungal pathogens |
| B-1,3-glucanase (antisense) | bacterial or fungal pathogens |
| AX + WIN-proteins | bacterial and fungal pathogens such as |
| | Cercospora beticola |
| receptor kinase | bacterial or fungal pathogens |
| polypeptide having the effect of triggering | bacterial or fungal pathogens |
| a hypersensitivity reaction | |
| systemic aquired resistance (SAR) genes | viral, bacterial, fungal and nematodal |
| | pathogens |
| lytic protein | bacterial or fungal pathogens |
| lysozyme | bacterial or fungal pathogens |
| chitinases | bacterial or fungal pathogens |
| barnase | bacterial or fungal pathogens |
| glucanases | bacterial or fungal pathogens |
| double-strand ribonuclease | viruses such as, for example, BNYVV |
| envelope proteins | viruses such as, for example, BNYVV |
| 17 kDa or 60 kDa protein | viruses such as, for example, BNYVV |
| nuclear inclusion proteins e.g. a or b or | viruses such as, for example, BNYVV |
| nucleoprotein | |
| pseudoubiquitin | viruses such as, for example, BNYVV |
| replicase | viruses such as, for example, BNYVV |
| toxins of Bacillus thuringiensis, VIP 3, | Lepidoptera, aphids, mites, nematodes, |
| Bacilluscereus toxins, Photorabdus and | whitefly, beetles, root-flies |
| Xenorhabdus toxins | |
| 3-hydroxysteroid oxidase | Lepidoptera, aphids, mites, nematodes, |
| | whitefly, beetles, root-flies |
| peroxidase | Lepidoptera, aphids, mites, nematodes, |
| | whitefly, beetles, root-flies |
| aminopeptidase inhibitors, e.g. leucine | Lepidoptera, aphids, mites, nematodes, |
| aminopeptidase inhibitor | whitefly, beetles, root-flies |
| lectins | Lepidoptera, aphids, mites, nematodes, |
| | whitefly, beetles, root-flies |
| protease inhibitors, e.g. cystatin, patatin, | Lepidoptera, aphids, mites, nematodes, |
| CPTI, virgiferin | whitefly, beetles, root-flies |
| ribosome-inactivating protein | Lepidoptera, aphids, mites, nematodes, |
| | whitefly, beetles, root-flies |
| stilbene synthase | Lepidoptera, aphids, mites, nematodes, |
| | whitefly, beetles, root-flies |
| HMG-CoA reductase | Lepidoptera, aphids, mites, nematodes, |
| | whitefly, beetles, root-flies |
| hatching factor for cyst nematodes | cyst nematodes |
| barnase | nematodes, e.g. root-knot nematodes and |
| | cyst nematodes |
| beet cyst nematode resistance site | cyst nematodes |
| CBI | root-knot nematodes |
| principles for preventing food uptake | nematodes, e.g. root-knot nematodes and |
| induced | root-cyst nematodes |

TABLE 2

| AP | Control of |
|---|---|
| CrylA(a) | Adoxophyes spp. |
| CrylA(a) | Agrotis spp. |
| CrylA(a) | Alabama argiliaceae |
| CrylA(a) | Anticarsia gemmatalis |
| CrylA(a) | Chilo spp. |
| CrylA(a) | Clysia ambiguella |
| CrylA(a) | Crocidolomia binotalis |
| CrylA(a) | Cydia spp. |
| CrylA(a) | Diparopsis castanea |
| CrylA(a) | Earias spp. |
| CrylA(a) | Ephestia spp. |
| CrylA(a) | Heliothis spp. |
| CrylA(a) | Hellula undalis |
| CrylA(a) | Keiferia lycopersicella |
| CrylA(a) | Leucoptera scitella |
| CrylA(a) | Lithocollethis spp. |
| CrylA(a) | Lobesia botrana |
| CrylA(a) | Ostrinia nubilalis |
| CrylA(a) | Pandemis spp. |
| CrylA(a) | Pectinophora gossyp. |
| CrylA(a) | Phyllocnistis citrella |
| CrylA(a) | Pieris spp. |
| CrylA(a) | Plutella xylostella |
| CrylA(a) | Scirpophaga spp. |
| CrylA(a) | Sesamia spp. |
| CrylA(a) | Sparganothis spp. |
| CrylA(a) | Spodoptera spp. |
| CrylA(a) | Tort-rix spp. |
| CrylA(a) | Trichoplusia ni |
| CrylA(a) | Agriotes spp. |
| CrylA(a) | Anthonomus grandis |
| CrylA(a) | Curculio spp. |
| CrylA(a) | Diabrotica balteata |
| CrylA(a) | Leptinotarsa spp. |
| CrylA(a) | Lissorhoptrus spp. |
| CrylA(a) | Otiorhynchus spp. |
| CrylA(a) | Aleurothrixus spp. |
| CrylA(a) | Aleyrodes spp. |
| CrylA(a) | Aonidiella spp. |
| CrylA(a) | Aphididea spp. |
| CrylA(a) | Aphis spp. |
| CrylA(a) | Bemisia tabaci |
| CrylA(a) | Empoasca spp. |
| CrylA(a) | Mycus spp. |
| CrylA(a) | Nephotettix spp. |
| CrylA(a) | Nilaparvata spp. |
| CrylA(a) | Pseudococcus spp. |
| CrylA(a) | Psylla spp. |
| CrylA(a) | Quadraspidiotus spp. |
| CrylA(a) | Schizaphis spp. |
| CrylA(a) | Trialeurodes spp. |
| CrylA(a) | Lyriomyza spp. |
| CrylA(a) | Oscinella spp. |
| CrylA(a) | Phorbia spp. |
| CrylA(a) | Frankliniella spp. |
| CrylA(a) | Thrips spp. |
| CrylA(a) | Scirtothrips aurantii |
| CrylA(a) | Aceria spp. |
| CrylA(a) | Aculus spp. |
| CrylA(a) | Brevipalpus spp. |
| CrylA(a) | Panonychus spp. |
| CrylA(a) | Phyllocoptruta spp. |
| CrylA(a) | Tetranychus spp. |
| CrylA(a) | Heterodera spp. |
| CrylA(a) | Meloidogyne spp. |
| CrylA(b) | Adoxophyes spp. |
| CrylA(b) | Agrotis spp. |
| CrylA(b) | Alabama argillaceae |
| CrylA(b) | Anticarsia gemmatalis |
| CrylA(b) | Chilo spp. |
| CrylA(b) | Clysia ambiguella |
| CrylA(b) | Crocidolomia binotalis |
| CrylA(b) | Cydia spp. |
| CrylA(b) | Diparopsis castanea |
| CrylA(b) | Earias spp. |
| CrylA(b) | Ephestia spp. |
| CrylA(b) | Heliothis spp. |
| CrylA(b) | Hellula undalis |
| CrylA(b) | Keiferia lycopersicella |
| CrylA(b) | Leucoptera scitella |
| CrylA(b) | Lithocollethis spp. |
| CrylA(b) | Lobesia botrana |
| CrylA(b) | Ostrinia nubilalis |
| CrylA(b) | Pandemis spp. |
| CrylA(b) | Pectinophora gossyp. |
| CrylA(b) | Phyllocnistis citrella |
| CrylA(b) | Pieris spp. |
| CrylA(b) | Plutelia xylostella |
| CrylA(b) | Scirpophaga spp. |
| CrylA(b) | Sesamia spp. |
| CrylA(b) | Sparganothis spp. |
| CrylA(b) | Spodoptera spp. |
| CrylA(b) | Tortrix spp. |
| CrylA(b) | Trichoplusia ni |
| CrylA(b) | Agriotes spp. |
| CrylA(b) | Anthonomus grandis |
| CrylA(b) | Curculio spp. |
| CrylA(b) | Diabrotica balteata |
| CrylA(b) | Leptinotarsa spp. |
| CrylA(b) | Lissorhoptrus spp. |
| CrylA(b) | Otiorhynchus spp. |
| CrylA(b) | Aleurothrixus spp. |
| CrylA(b) | Aleyrodes spp. |
| CrylA(b) | Aonidiella spp. |
| CrylA(b) | Aphididae spp. |
| CrylA(b) | Aphis spp. |
| CrylA(b) | Bemisia tabaci |
| CrylA(b) | Empoasca spp. |
| CrylA(b) | Mycus spp. |
| CrylA(b) | Nephotettix spp. |
| CrylA(b) | Nilaparvata spp. |
| CrylA(b) | Pseudococcus spp. |
| CrylA(b) | Psylla spp. |
| CrylA(b) | Quadraspidiotus spp. |
| CrylA(b) | Schizaphis spp. |
| CrylA(b) | Trialeurodes spp. |
| CrylA(b) | Lyriomyza spp. |
| CrylA(b) | Oscinella spp. |
| CrylA(b) | Phorbia spp. |
| CrylA(b) | Frankliniella spp. |
| CrylA(b) | Thrips spp. |
| CrylA(b) | Scirtothrips aurantii |
| CrylA(b) | Aceria spp. |
| CrylA(b) | Aculus spp. |
| CrylA(b) | Brevipalpus spp. |
| CrylA(b) | Panonychus spp. |
| CrylA(b) | Phyllocoptruta spp. |
| CrylA(b) | Tetranychus spp. |
| CrylA(b) | Heterodera spp. |
| CrylA(b) | Meloidogyne spp. |
| CrylA(c) | Adoxophyes spp. |
| CrylA(c) | Agrotis spp. |
| CrylA(c) | Alabama argillaceae |
| CrylA(c) | Anticarsia gemmatalis |
| CrylA(c) | Chilo spp. |
| CrylA(c) | Clysia ambiguella |
| CrylA(c) | Crocidolomia binotalis |
| CrylA(c) | Cydia spp. |
| CrylA(c) | Diparopsis castanea |
| CrylA(c) | Earias spp. |
| CrylA(c) | Ephestia spp. |
| CrylA(c) | Heliothis spp. |
| CrylA(c) | Hellula undalis |
| CrylA(c) | Keiferia lycopersicella |
| CrylA(c) | Leucoptera scitella |
| CrylA(c) | Lithocollethis spp. |
| CrylA(c) | Lobesia botrana |
| CrylA(c) | Ostrinia nubilalis |
| CrylA(c) | Pandemis spp. |
| CrylA(c) | Pectinophora gossypielia. |
| CrylA(c) | Phyllocnistis citrella |
| CrylA(c) | Pieris spp. |
| CrylA(c) | Plutella xylostella |
| CrylA(c) | Scirpophaga spp. |
| CrylA(c) | Sesamia spp. |
| CrylA(c) | Sparganothis spp. |
| CrylA(c) | Spodoptera spp. |
| CrylA(c) | Tortrix spp. |
| CrylA(c) | Trichoplusia ni |
| CrylA(c) | Agriotes spp. |
| CrylA(c) | Anthonomus grandis |
| CrylA(c) | Curculio spp. |
| CrylA(c) | Diabrotica balteata |
| CrylA(c) | Leptinotarsa spp. |
| CrylA(c) | Lissorhoptrus spp. |
| CrylA(c) | Otiorhynchus spp. |
| CrylA(c) | Aleurothrixus spp. |
| CrylA(c) | Aleyrodes spp. |
| CrylA(c) | Aonidiella spp. |
| CrylA(c) | Aphididae spp. |
| CrylA(c) | Aphis spp. |
| CrylA(c) | Bemisia tabaci |
| CrylA(c) | Empoasca spp. |
| CrylA(c) | Mycus spp. |
| CrylA(c) | Nephotettix spp. |
| CrylA(c) | Nilaparvata spp. |
| CrylA(c) | Pseudococcus spp. |
| CrylA(c) | Psylla spp. |
| CrylA(c) | Quadraspidiotus spp. |
| CrylA(c) | Schizaphis spp. |
| CrylA(c) | Trialeurodes spp. |
| CrylA(c) | Lyriomyza spp. |
| CrylA(c) | Oscinella spp. |
| CrylA(c) | Phorbia spp. |
| CrylA(c) | Frankliniella spp. |
| CrylA(c) | Thrips spp. |
| CrylA(c) | Scirtothrips aurantii |
| CrylA(c) | Aceria spp. |
| CrylA(c) | Aculus spp. |
| CrylA(c) | Brevipalpus spp. |
| CrylA(c) | Panonychus spp. |
| CrylA(c) | Phyllocoptruta spp. |
| CrylA(c) | Tetranychus spp. |
| CrylA(c) | Heterodera spp. |
| CrylA(c) | Meloidogyne spp. |
| CryllA | Adoxophyes spp. |
| CryllA | Agrotis spp. |
| CryllA | Alabama argillaceae |
| CryllA | Anticarsia gemmatalis |
| CryllA | Chilo spp. |
| CryllA | Clysia ambiguella |
| CryllA | Crocidolomia binotalis |
| CryllA | Cydia spp. |
| CryllA | Diparopsis castanea |
| CryllA | Earias spp. |
| CryllA | Ephestia spp. |
| CryllA | Heliothis spp. |
| CryllA | Hellula undalis |
| CryllA | Keiferia lycopersicella |
| CryllA | Leucoptera scitella |
| CryllA | Lithocoliethis spp. |
| CryllA | Lobesia botrana |
| CryllA | Ostrinia nubilalis |
| CryllA | Pandemis spp. |
| CryllA | Pectinophora gossyp. |
| CryllA | Phyllocnistis citrella |
| CryllA | Pieris spp. |
| CryllA | Plutella xylostella |
| CryllA | Scirpophaga spp. |
| CryllA | Sesamia spp. |
| CryllA | Sparganothis spp. |
| CryllA | Spodoptera spp. |
| CryllA | Tortrix spp. |
| CryllA | Trichoplusia ni |
| CryllA | Agriotes spp. |
| CryllA | Anthonomus grandis |
| CryllA | Curculio spp. |
| CryllA | Diabrotica balteata |
| CryllA | Leptinotarsa spp. |
| CryllA | Lissorhoptrus spp. |
| CryllA | Otiorhynchus spp. |
| CryllA | Aleurothrixus spp. |
| CryllA | Aleyrodes spp. |
| CryllA | Aonidiella spp. |
| CryllA | Aphididae spp. |
| CryllA | Aphis spp. |
| CryllA | Bemisia tabaci |
| CryllA | Empoasca spp. |
| CryllA | Mycus spp. |
| CryllA | Nephotettix spp. |
| CryllA | Nilaparvata spp. |
| CryllA | Pseudococcus spp. |
| CryllA | Psylla spp. |
| CryllA | Quadraspidiotus spp. |
| CryllA | Schizaphis spp. |
| CryllA | Trialeurodes spp. |
| CryllA | Lyriomyza spp. |
| CryllA | Oscinella spp. |
| CryllA | Phorbia spp. |
| CryllA | Frankliniella spp. |
| CryllA | Thrips spp. |
| CryllA | Scirtothrips aurantii |
| CryllA | Aceria spp. |
| CryllA | Acutus spp. |
| CryllA | Brevipalpus spp. |
| CryllA | Panonychus spp. |
| CryllA | Phyllocoptruta spp. |
| CryllA | Tetranychus spp. |
| CryllA | Heterodera spp. |
| CryllA | Meloidogyne spp. |
| CrylllA | Adoxophyes spp. |
| CrylllA | Agrotis spp. |
| CrylllA | Alabama argillaceae |
| CrylllA | Anticarsia gemmatalis |
| CrylllA | Chilo spp. |
| CrylllA | Clysia ambiguelia |
| CrylllA | Crocodolomia binotalis |
| CrylllA | Cydia spp. |
| CrylllA | Diparopsis castanea |
| CrylllA | Earias spp. |
| CrylllA | Ephestia spp. |
| CrylllA | Heliothis spp. |
| CrylllA | Hellula undalis |
| CrylllA | Keiferia lycopersicella |
| CrylllA | Leucoptera scitella |
| CrylllA | Lithocollethis spp. |
| CrylllA | Lobesia botrana |
| CrylllA | Ostrinia nubilalis |
| CrylllA | Pandemis spp. |
| CrylllA | Pectinophora gossyp. |
| CrylllA | Phyllocnistis citrella |
| CrylllA | Pieris spp. |
| CrylllA | Plutella xylostella |
| CrylllA | Scirpophaga spp. |
| CrylllA | Sesamia spp. |
| CrylllA | Sparganothis spp. |
| CrylllA | Spodoptera spp. |
| CrylllA | Tortrix spp. |
| CrylllA | Trichoplusia ni |
| CrylllA | Agriotes spp. |
| CrylllA | Anthonomus grandis |
| CrylllA | Curculio spp. |
| CrylllA | Diabrotica balteata |
| CrylllA | Leptinotarsa spp. |
| CrylllA | Lissorhoptrus spp. |
| CrylllA | Otiorhynchus spp. |
| CrylllA | Aleurothrixus spp. |
| CrylllA | Aleyrodes spp. |
| CrylllA | Aonidiella spp. |
| CrylllA | Aphididae spp. |
| CrylllA | Aphis spp. |
| CrylllA | Bemisia tabaci |
| CrylllA | Empoasca spp. |
| CrylllA | Mycus spp. |
| CrylllA | Nephotettix spp. |
| CrylllA | Nilaparvata spp. |
| CrylllA | Pseudococcus spp. |
| CrylllA | Psylla spp. |
| CrylllA | Quadraspidiotus spp. |
| CrylllA | Schizaphis spp. |
| CrylllA | Trialeurodes spp. |
| CrylllA | Lyriomyza spp. |
| CrylllA | Oscinella spp. |
| CrylllA | Phorbia spp. |
| CrylllA | Frankliniella spp. |
| CrylllA | Thrips spp. |
| CrylllA | Scirtothrips aurantii |
| CrylllA | Aceria spp. |
| CrylllA | Aculus spp. |
| CrylllA | Brevipalpus spp. |
| CrylllA | Panonychus spp. |
| CrylllA | Phyllocoptruta spp. |
| CrylllA | Tetranychus spp. |
| CrylllA | Heterodera spp. |
| CrylllA | Meloidogyne spp. |
| CrylllB2 | Adoxophyes spp. |
| CrylllB2 | Agrotis spp. |
| CrylllB2 | Alabama argillaceae |
| CrylllB2 | Anticarsia gemmatalis |
| CrylllB2 | Chilo spp. |
| CrylllB2 | Clysia ambiguella |
| CrylllB2 | Crocidolomia binotalis |
| CrylllB2 | Cydia spp. |
| CrylllB2 | Diparopsis castanea |
| CrylllB2 | Earias spp. |
| CrylllB2 | Ephestia spp. |
| CrylllB2 | Heliothis spp. |
| CrylllB2 | Hellula undalis |
| CrylllB2 | Keiferia lycopersicella |
| CrylllB2 | Leucoptera scitella |
| CrylllB2 | Lithocollethis spp. |
| CrylllB2 | Lobesia botrana |
| CrylllB2 | Ostrinia nubilalis |
| CrylllB2 | Pandemis spp. |
| CrylllB2 | Pectinophora gossyp. |
| CrylllB2 | Phyllocnistis citrella |
| CrylllB2 | Pieris spp. |
| CrylllB2 | Plutella xylostella |
| CrylllB2 | Scirpophaga spp. |
| CrylllB2 | Sesamia spp. |
| CrylllB2 | Sparganothis spp. |
| CrylllB2 | Spodoptera spp. |
| CrylllB2 | Tortrix spp. |
| CrylllB2 | Trichoplusia ni |
| CrylllB2 | Agriotes spp. |
| CrylllB2 | Anthonomus grandis |
| CrylllB2 | Curculio spp. |
| CrylllB2 | Diabrotica balteata |
| CrylllB2 | Leptinotarsa spp. |
| CrylllB2 | Lissorhoptrus spp. |
| CrylllB2 | Otiorhynchus spp. |
| CrylllB2 | Aleurothrixus spp. |
| CrylllB2 | Aleyrodes spp. |
| CrylllB2 | Aonidiella spp. |
| CrylllB2 | Aphididae spp. |
| CrylllB2 | Aphis spp. |
| CrylllB2 | Bemisia tabaci |
| CrylllB2 | Empoasca spp. |
| CrylllB2 | Mycus spp. |
| CrylllB2 | Nephotettix spp. |
| CrylllB2 | Nilaparvata spp. |
| CrylllB2 | Pseudococcus spp. |
| CrylllB2 | Psylla spp. |
| CrylllB2 | Quadraspidiotus spp. |
| CrylllB2 | Schizaphis spp. |
| CrylllB2 | Trialeurodes spp. |
| CrylllB2 | Lyriornyza spp. |
| CrylllB2 | Oscinella spp. |
| CrylllB2 | Phorbia spp. |
| CrylllB2 | Frankliniella spp. |
| CrylllB2 | Thrips spp. |
| CrylllB2 | Scirtothrips aurantii |
| CrylllB2 | Aceria spp. |
| CrylllB2 | Acutus spp. |
| CrylllB2 | Brevipalpus spp. |
| CrylllB2 | Panonychus spp. |
| CrylllB2 | Phyllocoptruta spp. |
| CrylllB2 | Tetranychus spp. |
| CrylllB2 | Heterodera spp. |
| CrylllB2 | Meloidogyne spp. |
| CytA | Adoxophyes spp. |
| CytA | Agrotis spp. |
| CytA | Alabama argillaceae |
| CytA | Anticarsia gemmatalis |
| CytA | Chilo spp. |
| CytA | Clysia ambiguella |
| CytA | Crocidolomia binotalis |
| CytA | Cydia spp. |
| CytA | Diparopsis castanea |
| CytA | Earias spp. |
| CytA | Ephestia spp. |
| CytA | Heliothis spp. |
| CytA | Hellula undalis |
| CytA | Keiferia lycopersicella |
| CytA | Leucoptera scitella |
| CytA | Lithocollethis spp. |
| CytA | Lobesia botrana |
| CytA | Ostrinia nubilalis |
| CytA | Pandemis spp. |
| CytA | Pectinophora gossyp. |
| CytA | Phyllocnistis citrella |
| CytA | Pieris spp. |
| CytA | Plutella xylostella |
| CytA | Scirpophaga spp. |
| CytA | Sesamia spp. |
| CytA | Sparganothis spp. |
| CytA | Spodoptera spp. |
| CytA | Tortrix spp. |
| CytA | Trichoplusia ni |
| CytA | Agriotes spp. |
| CytA | Anthonomus grandis |
| CytA | Curculio spp. |
| CytA | Diabrotica balteata |
| CytA | Leptinotarsa spp. |
| CytA | Lissorhoptrus spp. |
| CytA | Otiorhynchus spp. |
| CytA | Aleurothrixus spp. |
| CytA | Aleyrodes spp. |
| CytA | Aonidiella spp. |
| CytA | Aphididae spp. |
| CytA | Aphis spp. |
| CytA | Bemisia tabaci |
| CytA | Empoasca spp. |
| CytA | Mycus spp. |
| CytA | Nephotettix spp. |
| CytA | Nilaparvata spp. |
| CytA | Pseudococcus spp. |
| CytA | Psylla spp. |
| CytA | Quadraspidiotus spp. |
| CytA | Schizaphis spp. |
| CytA | Trialeurodes spp. |
| CytA | Lyriomyza spp. |
| CytA | Oscinella spp. |
| CytA | Phorbia spp. |
| CytA | Frankliniella spp. |
| CytA | Thrips spp. |
| CytA | Scirtothrips aurantii |
| CytA | Aceria spp. |
| CytA | Acutus spp. |
| CytA | Brevipalpus spp. |
| CytA | Panonychus spp. |
| CytA | Phyllocoptruta spp. |
| CytA | Tetranychus spp. |
| CytA | Heterodera spp. |
| CytA | Meloidogyne spp. |
| VIP3 | Adoxophyes spp. |
| VIP3 | Agrotis spp. |
| VIP3 | Alabama argillaceae |
| VIP3 | Anticarsia gemmatalis |
| VIP3 | Chilo spp. |
| VIP3 | Clysia ambiguella |
| VIP3 | Crocidolomia binotalis |
| VIP3 | Cydia spp. |
| VIP3 | Diparopsis castanea |
| VIP3 | Earias spp. |
| VIP3 | Ephestia spp. |
| VIP3 | Heliothis spp. |
| VIP3 | Hellula undalis |
| VIP3 | Keiferia |
| | lycopersicella |
| VIP3 | Leucoptera scitella |
| VIP3 | Lithocollethis spp. |
| VIP3 | Lobesia botrana |
| VIP3 | Ostrinia nubilalis |
| VIP3 | Pandemis spp. |
| VIP3 | Pectinophora gossyp. |
| VIP3 | Phyllocnistis citrella |
| VIP3 | Pieris spp. |
| VIP3 | Plutella xylostella |
| VIP3 | Scirpophaga spp. |
| VIP3 | Sesamia spp. |
| VIP3 | Sparganothis spp. |
| VIP3 | Spodoptera spp. |
| VIP3 | Tortrix spp. |
| VIP3 | Trichoplusia ni |
| VIP3 | Agriotes spp. |
| VIP3 | Anthonomus grandis |
| VIP3 | Curculio spp. |
| VIP3 | Diabrotica balteata |
| VIP3 | Leptinotarsa spp. |
| VIP3 | Lissorhoptrus spp. |
| VIP3 | Otiorhynchus spp. |
| VIP3 | Aleurothrixus spp. |
| VIP3 | Aleyrodes spp. |
| VIP3 | Aonidiella spp. |
| VIP3 | Aphididae spp. |
| VIP3 | Aphis spp. |
| VIP3 | Bemisia tabaci |
| VIP3 | Empoasca spp. |
| VIP3 | Mycus spp. |
| VIP3 | Nephotettix spp. |
| VIP3 | Nilaparvata spp. |
| VIP3 | Pseudococcus spp. |
| VIP3 | Psylla spp. |
| VIP3 | Quadraspidiotus spp. |
| VIP3 | Schizaphis spp. |
| VIP3 | Trialeurodes spp. |
| VIP3 | Lyriomyza spp. |
| VIP3 | Oscinella spp. |
| VIP3 | Phorbia spp. |
| VIP3 | Frankliniella spp. |
| VIP3 | Thrips spp. |
| VIP3 | Scirtothrips aurantii |
| VIP3 | Aceria spp. |
| VIP3 | Acutus spp. |
| VIP3 | Brevipalpus spp. |
| VIP3 | Panonychus spp. |
| VIP3 | Phyllocoptruta spp. |
| VIP3 | Tetranychus spp. |
| VIP3 | Heterodera spp. |
| VIP3 | Meloidogyne spp. |
| GL | Adoxophyes spp. |
| GL | Agrotis spp. |
| GL | Alabama argillaceae |
| GL | Anticarsia gemmatalis |
| GL | Chilo spp. |
| GL | Clysia ambiguella |
| GL | Crocidolomia binotalis |
| GL | Cydia spp. |
| GL | Diparopsis castanea |
| GL | Earias spp. |
| GL | Ephestia spp. |
| GL | Heliothis spp. |
| GL | Hellula undalis |
| GL | Keiferia lycopersicella |
| GL | Leucoptera scitella |
| GL | Lithocollethis spp. |
| GL | Lobesia botrana |
| GL | Ostrinia nubilalis |
| GL | Pandemis spp. |
| GL | Pectinophora gossyp. |
| GL | Phyliocnistis citrella |
| GL | Pieris spp. |
| GL | Plutella xylostella |
| GL | Scirpophaga spp. |
| GL | Sesamia spp. |
| GL | Sparganothis spp. |
| GL | Spodoptera spp. |
| GL | Tortrix spp. |
| GL | Trichoplusia ni |
| GL | Agriotes spp. |
| GL | Anthonomus grandis |
| GL | Curculio spp. |
| GL | Diabrotica balteata |
| GL | Leptinotarsa spp. |
| GL | Lissorhoptrus spp. |
| GL | Otiorhynchus spp. |
| GL | Aleurothrixus spp. |
| GL | Aleyrodes spp. |
| GL | Aonidiella spp. |
| GL | Aphididae spp. |
| GL | Aphis spp. |
| GL | Bemisia tabaci |
| GL | Empoasca spp. |
| GL | Mycus spp. |
| GL | Nephotettix spp. |
| GL | Nilaparvata spp. |
| GL | Pseudococcus spp. |
| GL | Psylia spp. |
| GL | Quadraspidiotus spp. |
| GL | Schizaphis spp. |
| GL | Trialeurodes spp. |
| GL | Lyriomyza spp. |
| GL | Oscinella spp. |
| GL | Phorbia spp. |
| GL | Frankliniella spp. |
| GL | Thrips spp. |
| GL | Scirtothrips aurantii |
| GL | Aceria spp. |
| GL | Aculus spp. |
| GL | Brevipalpus spp. |
| GL | Panonychus spp. |
| GL | Phyliocoptruta spp. |
| GL | Tetranychus spp. |
| GL | Heterodera spp. |
| GL | Meloidogyne spp. |
| PL | Adoxophyes spp. |
| PL | Agrotis spp. |
| PL | Alabama argillaceae |
| PL | Anticarsia gemmatalis |
| PL | Chilo spp. |
| PL | Clysia ambiguella |
| PL | Crocidolomia binotalis |
| PL | Cydia spp. |
| PL | Diparopsis castanea |
| PL | Earias spp. |
| PL | Ephestia spp. |
| PL | Heliothis spp. |
| PL | Hellula undalis |
| PL | Keiferia lycopersicella |
| PL | Leucoptera scitella |
| PL | Lithocollethis spp. |
| PL | Lobesia botrana |
| PL | Ostrinia nubilalis |
| PL | Pandemis spp. |
| PL | Pectinophora gossyp. |
| PL | Phyllocnistis citrella |
| PL | Pieris spp. |
| PL | Plutella xylostella |
| PL | Scirpophaga spp. |
| PL | Sesamia spp. |
| PL | Sparganothis spp. |
| PL | Spodoptera spp. |
| PL | Tortrix spp. |
| PL | Trichoplusia ni |
| PL | Agriotes spp. |
| PL | Anthonomus grandis |
| PL | Curculio spp. |
| PL | Diabrotica balteata |
| PL | Leptinotarsa spp. |
| PL | Lissorhoptrus spp. |
| PL | Otiorhynchus spp. |
| PL | Aleurothrixus spp. |
| PL | Aleyrodes spp. |
| PL | Aonidiella spp. |
| PL | Aphididae spp. |
| PL | Aphis spp. |
| PL | Bemisia tabaci |
| PL | Empoasca spp. |
| PL | Mycus spp. |
| PL | Nephotettix spp. |
| PL | Nilaparvata spp. |
| PL | Pseudococcus spp. |
| PL | Psylla spp. |
| PL | Quadraspidiotus spp. |
| PL | Schizaphis spp. |
| PL | Trialeurodes spp. |
| PL | Lyriomyza spp. |
| PL | Oscinella spp. |
| PL | Phorbia spp. |
| PL | Frankliniella spp. |
| PL | Thrips spp. |
| PL | Scirtothrips aurantii |
| PL | Aceria spp. |
| PL | Aculus spp. |
| PL | Brevipalpus spp. |
| PL | Panonychus spp. |
| PL | Phyllocoptruta spp. |
| PL | Tetranychus spp. |
| PL | Heterodera spp. |
| PL | Meloidogyne spp. |
| XN | Adoxophyes spp. |
| XN | Agrotis spp. |
| XN | Alabama argillaceae |
| XN | Anticarsia gemmatalis |
| XN | Chilo spp. |
| XN | Clysia ambiguella |
| XN | Crocidolomia binotalis |
| XN | Cydia spp. |
| XN | Diparopsis castanea |
| XN | Earias spp. |
| XN | Ephestia spp. |
| XN | Heliothis spp. |
| XN | Hellula undalis |
| XN | Keiferia lycopersicella |
| XN | Leucoptera scitella |
| XN | Lithocollethis spp. |
| XN | Lobesia botrana |
| XN | Ostrinia nubilalis |
| XN | Pandemis spp. |
| XN | Pectinophora gossyp. |
| XN | Phyllocnistis citrella |
| XN | Pieris spp. |
| XN | Plutella xylostella |
| XN | Scirpophaga spp. |
| XN | Sesamia spp. |
| XN | Sparganothis spp. |
| XN | Spodoptera spp. |
| XN | Tortrix spp. |
| XN | Trichoplusia ni |
| XN | Agriotes spp. |
| XN | Anthonomus grandis |
| XN | Curculio spp. |
| XN | Diabrotica balteata |
| XN | Leptinotarsa spp. |
| XN | Lissorhoptrus spp. |
| XN | Otiorhynchus spp. |
| XN | Aleurothrixus spp. |
| XN | Aleyrodes spp. |
| XN | Aonidiella spp. |
| XN | Aphididae spp. |
| XN | Aphis spp. |
| XN | Bemisia tabaci |
| XN | Empoasca spp. |
| XN | Mycus spp. |
| XN | Nephotettix spp. |
| XN | Nilaparvata spp. |
| XN | Pseudococcus spp. |
| XN | Psylla spp. |
| XN | Quadraspidiotus spp. |
| XN | Schizaphis spp. |
| XN | Trialeurodes spp. |
| XN | Lyriomyza spp. |
| XN | Oscinella spp. |
| XN | Phorbia spp. |
| XN | Frankliniella spp. |
| XN | Thrips spp. |
| XN | Scirtothrips aurantii |
| XN | Aceria spp. |
| XN | Aculus spp. |
| XN | Brevipalpus spp. |
| XN | Panonychus spp. |
| XN | Phyllocoptruta spp. |
| XN | Tetranychus spp. |
| XN | Heterodera spp. |
| XN | Meloidogyne spp. |
| Plnh. | Adoxophyes spp. |
| Plnh. | Agrotis spp. |
| Plnh. | Alabama argillaceae |
| Plnh. | Anticarsia gemmatalis |
| Plnh. | Chilo spp. |
| Plnh. | Clysia ambiguella |
| Plnh. | Crocidolomia |
| | binotalis |
| Plnh. | Cydia spp. |
| Plnh. | Diparopsis castanea |
| Plnh. | Earias spp. |
| Plnh. | Ephestia spp. |
| Plnh. | Heliothis spp. |
| Plnh. | Hellula undalis |
| Plnh. | Keiferia lycopersicella |
| Plnh. | Leucoptera scitella |
| Plnh. | Lithocollethis spp. |
| Plnh. | Lobesia botrana |
| Plnh. | Ostrinia nubilalis |
| Plnh. | Pandemis spp. |
| Plnh. | Pectinophora gossyp. |
| Plnh. | Phyllocnistis citrelia |
| Plnh. | Pieris spp. |
| Plnh. | Plutella xylostella |
| Plnh. | Scirpophaga spp. |
| Plnh. | Sesamia spp. |
| Plnh. | Sparganothis spp. |
| Plnh. | Spodoptera spp. |
| Plnh. | Tortrix spp. |
| Plnh. | Trichoplusia ni |
| Plnh. | Agriotes spp. |
| Plnh. | Anthonomus grandis |
| Plnh. | Curculio spp. |
| Plnh. | Diabrotica balteata |
| Plnh. | Leptinotarsa spp. |
| Plnh. | Lissorhoptrus spp. |
| Plnh. | Otiorhynchus spp. |
| Plnh. | Aleurothrixus spp. |
| Plnh. | Aleyrodes spp. |
| Plnh. | Aonidiella spp. |
| Plnh. | Aphididae spp. |
| Plnh. | Aphis spp. |
| Plnh. | Bemisia tabaci |
| Plnh. | Empoasca spp. |
| Plnh. | Mycus spp. |
| Plnh. | Nephotettix spp. |
| Plnh. | Nilaparvata spp. |
| Plnh. | Pseudococcus spp. |
| Plnh. | Psylla spp. |
| Plnh. | Quadraspidiotus spp. |
| Plnh. | Schizaphis spp. |
| Plnh. | Trialeurodes spp. |
| Plnh. | Lyriomyza spp. |
| Plnh. | Oscinella spp. |
| Plnh. | Phorbia spp. |
| Plnh. | Frankliniella spp. |
| Plnh. | Thrips spp. |
| Plnh. | Scirtothrips aurantii |
| Plnh. | Aceria spp. |
| Plnh. | Acutus spp. |
| Plnh. | Brevipalpus spp. |
| Plnh. | Panonychus spp. |
| Plnh. | Phyllocoptruta spp. |
| Plnh. | Tetranychus spp. |
| Plnh. | Heterodera spp. |
| Plnh. | Meloidogyne spp. |
| PLec. | Adoxophyes spp. |
| PLec. | Agrotis spp. |
| PLec. | Alabama argillaceae |
| PLec. | Anticarsia gemmatalis |
| PLec. | Chilo spp. |
| PLec. | Clysia ambiguella |
| PLec. | Crocidolomia binotalis |
| PLec. | Cydia spp. |
| PLec. | Diparopsis castanea |
| PLec. | Earias spp. |
| PLec. | Ephestia spp. |
| PLec. | Heliothis spp. |
| PLec. | Hellula undalis |
| PLec. | Keiferia lycopersicella |
| PLec. | Leucoptera scitella |
| PLec. | Lithocollethis spp. |
| PLec. | Lobesia botrana |
| PLec. | Ostrinia nubilalis |
| PLec. | Pandemis spp. |
| PLec. | Pectinophora gossyp. |
| PLec. | Phyllocnistis citrella |
| PLec. | Pieris spp. |
| PLec. | Plutella xylostella |
| PLec. | Scirpophaga spp. |
| PLec. | Sesamia spp. |
| PLec. | Sparganothis spp. |
| PLec. | Spodoptera spp. |
| PLec. | Tortrix spp. |
| PLec. | Trichoplusia ni |
| PLec. | Agriotes spp. |
| PLec. | Anthonomus grandis |
| PLec. | Curculio spp. |
| PLec. | Diabrotica balteata |
| PLec. | Leptinotarsa spp. |
| PLec. | Lissorhoptrus spp. |
| PLec. | Otiorhynchus spp. |
| PLec. | Aleurothrixus spp. |
| PLec. | Aleyrodes spp. |
| PLec. | Aonidiella spp. |
| PLec. | Aphididae spp. |
| PLec. | Aphis spp. |
| PLec. | Bemisia tabaci |
| PLec. | Empoasca spp. |
| PLec. | Mycus spp. |
| PLec. | Nephotettix spp. |
| PLec. | Nilaparvata spp. |
| PLec. | Pseudococcus spp. |
| PLec. | Psylla spp. |
| PLec. | Quadraspidiotus spp. |
| PLec. | Schizaphis spp. |
| PLec. | Trialeurodes spp. |
| PLec. | Lyriomyza spp. |
| PLec. | Oscinella spp. |
| PLec. | Phorbia spp. |
| PLec. | Frankliniella spp. |
| PLec. | Thrips spp. |
| PLec. | Scirtothrips aurantii |
| PLec. | Aceria spp. |
| PLec. | Aculus spp. |
| PLec. | Brevipalpus spp. |
| PLec. | Panonychus spp. |
| PLec. | Phyllocoptruta spp. |
| PLec. | Tetranychus spp. |
| PLec. | Heterodera spp. |
| PLec. | Meloidogyne spp. |
| Aggl. | Adoxophyes spp. |
| Aggl. | Agrotis spp. |
| Aggl. | Alabama |
| | argillaceae |
| Aggl. | Anticarsia gemmatalis |
| Aggl. | Chilo spp. |
| Aggl. | Clysia ambiguella |
| Aggl. | Crocidolomia |
| | binotalis |
| Aggl. | Cydia spp. |
| Aggl. | Diparopsis |
| | castanea |
| Aggl. | Earias spp. |
| Aggl. | Ephestia spp. |
| Aggl. | Heliothis spp. |
| Aggl. | Hellula undalis |
| Aggl. | Keiferia |
| | lycopersicella |
| Aggl. | Leucoptera scitella |
| Aggl. | Lithocollethis spp. |
| Aggl. | Lobesia botrana |
| Aggl. | Ostrinia nubilalis |
| Aggl. | Pandemis spp. |
| Aggl. | Pectinophora |
| | gossyp. |
| Aggl. | Phyllocnistis citrella |
| Aggl. | Pieris spp. |
| Aggl. | Plutella xylostella |
| Aggl. | Scirpophaga spp. |
| Aggl. | Sesamia spp. |
| Aggl. | Sparganothis spp. |
| Aggl. | Spodoptera spp. |
| Aggl. | Tortrix spp. |
| Aggl. | Trichoplusia ni |
| Aggl. | Agriotes spp. |
| Aggl. | Anthonomus grandis |
| Aggl. | Curculio spp. |
| Aggl. | Diabrotica balteata |
| Aggl. | Leptinotarsa spp. |
| Aggl. | Lissorhoptrus spp. |
| Aggl. | Otiorhynchus spp. |
| Aggl. | Aleurothrixus spp. |
| Aggl. | Aleyrodes spp. |
| Aggl. | Aonidiella spp. |
| Aggl. | Aphididae spp. |
| Aggl. | Aphis spp. |
| Aggl. | Bemisia tabaci |
| Aggl. | Empoasca spp. |
| Aggl. | Mycus spp. |
| Aggl. | Nephotettix spp. |
| Aggl. | Nilaparvata spp. |
| Aggl. | Pseudococcus spp. |
| Aggl. | Psylla spp. |
| Aggl. | Quadraspidiotus spp. |
| Aggl. | Schizaphis spp. |
| Aggl. | Trialeurodes spp. |
| Aggl. | Lyriomyza spp. |
| Aggl. | Oscinella spp. |
| Aggl. | Phorbia spp. |
| Aggl. | Frankliniella spp. |
| Aggl. | Thrips spp. |
| Aggl. | Scirtothrips aurantii |
| Aggl. | Aceria spp. |
| Aggl. | Aculus spp. |
| Aggl. | Brevipalpus spp. |
| Aggl. | Panonychus spp. |
| Aggl. | Phyllocoptruta spp |
| Aggl. | Tetranychus spp. |
| Aggl. | Heterodera spp. |
| Aggl. | Meloidogyne spp. |
| CO | Adoxophyes spp. |
| CO | Agrotis spp. |
| CO | Alabama argillaceae |
| CO | Anticarsia gemmatalis |
| CO | Chilo spp. |
| CO | Clysia ambiguella |
| CO | Crocidolomia binotalis |
| CO | Cydia spp. |
| CO | Diparopsis castanea |
| CO | Earias spp. |
| CO | Ephestia spp. |
| CO | Heliothis spp. |
| CO | Hellula undalis |
| CO | Keiferia lycopersicella |
| CO | Leucoptera scitella |
| CO | Lithocollethis spp. |
| CO | Lobesia botrana |
| CO | Ostrinia nubilalis |
| CO | Pandemis spp. |
| CO | Pectinophora gossyp. |
| CO | Phyllocnistis citrella |
| CO | Pieris spp. |
| CO | Plutella xylostella |
| CO | Scirpophaga spp. |
| CO | Sesamia spp. |
| CO | Sparganothis spp. |
| CO | Spodoptera spp. |
| CO | Tortrix spp. |
| CO | Trichoplusia ni |
| CO | Agriotes spp. |
| CO | Anthonomus grandis |
| CO | Curculio spp. |
| CO | Diabrotica balteata |
| CO | Leptinotarsa spp. |
| CO | Lissorhoptrus spp. |
| CO | Otiorhynchus spp. |
| CO | Aleurothrixus spp. |
| CO | Aleyrodes spp. |
| CO | Aonidiella spp. |
| CO | Aphididae spp. |
| CO | Aphis spp. |
| CO | Bemisia tabaci |
| CO | Empoasca spp. |
| CO | Mycus spp. |
| CO | Nephotettix spp. |
| CO | Nilaparvata spp. |
| CO | Pseudococcus spp. |
| CO | Psylla spp. |
| CO | Quadraspidiotus spp. |
| CO | Schizaphis spp. |
| CO | Trialeurodes spp. |
| CO | Lyriomyza spp. |
| CO | Oscinella spp. |
| CO | Phorbia spp. |
| CO | Frankliniella spp. |
| CO | Thrips spp. |
| CO | Scirtothrips aurantii |
| CO | Aceria spp. |
| CO | Acutus spp. |
| CO | Brevipalpus spp. |
| CO | Panonychus spp. |
| CO | Phyllocoptruta spp. |
| CO | Tetranychus spp. |
| CO | Heterodera spp. |
| CO | Meloidogyne spp. |
| CH | Adoxophyes spp. |
| CH | Agrotis spp. |
| CH | Alabama argillaceae |
| CH | Anticarsia |
| | gemmatalis |
| CH | Chilo spp. |
| CH | Clysia ambiguella |
| CH | Crocidolomia binotalis |
| CH | Cydia spp. |
| CH | Diparopsis castanea |
| CH | Earias spp. |
| CH | Ephestia spp. |
| CH | Heliothis spp. |
| CH | Hellula undalis |
| CH | Keiferia lycopersicella |
| CH | Leucoptera scitella |
| CH | Lithocollethis spp. |
| CH | Lobesia botrana |
| CH | Ostrinia nubilalis |
| CH | Pandemis spp. |
| CH | Pectinophora gossyp. |
| CH | Phyllocnistis citrella |
| CH | Pieris spp. |
| CH | Plutella xylostella |
| AP | Control of |
| CH | Scirpophaga spp. |
| CH | Sesamia spp. |
| CH | Sparganothis spp. |
| CH | Spodoptera spp. |
| CH | Tortrix spp. |
| CH | Trichoplusia ni |
| CH | Agriotes spp. |
| CH | Anthonomus |
| | grandis |
| CH | Curculio spp. |
| CH | Diabrotica balteata |
| CH | Leptinotarsa spp. |
| CH | Lissorhoptrus spp. |
| CH | Otiorhynohus spp. |
| CH | Aleurothrixus spp. |
| CH | Aleyrodes spp. |
| CH | Aonidiella spp. |
| CH | Aphididae spp. |
| CH | Aphis spp. |
| CH | Bemisia tabaci |
| CH | Empoasca spp. |
| CH | Mycus spp. |
| CH | Nephotettix spp. |
| CH | Nilaparvata spp. |
| CH | Pseudococcus spp. |
| CH | Psylla spp. |
| CH | Quadraspidiotus spp. |
| CH | Schizaphis spp. |
| CH | Trialeurodes spp. |
| CH | Lyriomyza spp. |
| CH | Oscinella spp. |
| CH | Phorbia spp. |
| CH | Frankliniella spp. |
| CH | Thrips spp. |
| CH | Scirtothrips aurantii |
| CH | Aceria spp. |
| CH | Aculus spp. |
| CH | Brevipalpus spp. |
| CH | Panonychus spp. |
| CH | Phyllocoptruta spp. |
| CH | Tetranychus spp. |
| CH | Heterodera spp. |
| CH | Meloidogyne spp. |
| SS | Adoxophyes spp. |
| SS | Agrotis spp. |
| SS | Alabama argillaceae |
| SS | Anticarsia gemmatalis |
| SS | Chilo spp. |
| SS | Clysia ambiguella |
| SS | Crocidolomia binotalis |
| SS | Cydia spp. |
| SS | Diparopsis castanea |
| SS | Earias spp. |
| SS | Ephestia spp. |
| SS | Heliothis spp. |
| SS | Hellula undalis |
| SS | Keiferia lycopersicella |
| SS | Leucoptera scitella |
| SS | Lithocollethis spp. |
| SS | Lobesia botrana |
| SS | Ostrinia nubilalis |
| SS | Pandemis spp. |
| SS | Pectinophora gossyp. |
| SS | Phyllocnistis citrella |
| SS | Pieris spp. |
| SS | Plutella xylostella |
| SS | Scirpophaga spp. |
| SS | Sesamia spp. |
| SS | Sparganothis spp. |
| SS | Spodoptera spp. |
| SS | Tortrix spp. |
| SS | Trichoplusia ni |
| SS | Agriotes spp. |
| SS | Anthonomus grandis |
| SS | Curculio spp. |
| SS | Diabrotica balteata |
| SS | Leptinotarsa spp. |
| SS | Lissorhoptrus spp. |
| SS | Otiorhynchus spp. |
| SS | Aleurothrixus spp. |
| SS | Aleyrodes spp. |
| SS | Aonidiella spp. |
| SS | Aphididae spp. |
| SS | Aphis spp. |
| SS | Bemisia tabaci |
| SS | Empoasca spp. |
| SS | Mycus spp. |
| SS | Nephotettix spp. |
| SS | Nilaparvata spp. |
| SS | Pseudococcus spp. |
| SS | Psylla spp. |
| SS | Quadraspidiotus spp. |
| SS | Schizaphis spp. |
| SS | Trialeurodes spp. |
| SS | Lyriomyza spp. |
| SS | Oscinella spp. |
| SS | Phorbia spp. |
| SS | Frankliniella spp. |
| SS | Thrips spp. |
| SS | Scirtothrips aurantii |
| SS | Aceria spp. |
| SS | Aculus spp. |
| SS | Brevipalpus spp. |
| SS | Panonychus spp. |
| SS | Phyllocoptruta spp. |
| SS | Tetranychus spp. |
| SS | Heterodera spp. |
| SS | Meloidogyne spp. |
| HO | Adoxophyes spp. |
| HO | Agrotis spp. |
| HO | Alabama argillaceae |
| HO | Anticarsia gemmatalis |
| HO | Chilo spp. |
| HO | Clysia ambiguella |
| HO | Crocidolomia binotalis |
| HO | Cydia spp. |
| HO | Diparopsis castanea |
| HO | Earias spp. |
| HO | Ephestia spp. |
| HO | Heliothis spp. |
| HO | Hellula undalis |
| HO | Keiferia lycopersicella |
| HO | Leucoptera scitella |
| HO | Lithocollethis spp. |
| HO | Lobesia botrana |
| HO | Ostrinia nubilalis |
| HO | Pandemis spp. |
| HO | Pectinophora gossypiella |
| HO | Phyllocnistis citrella |
| HO | Pieris spp. |
| HO | Plutella xylostella |
| HO | Scirpophaga spp. |
| HO | Sesamia spp. |
| HO | Sparganothis spp. |
| HO | Spodoptera spp. |
| HO | Tortrix spp. |
| HO | Trichoplusia ni |
| HO | Agriotes spp. |
| HO | Anthonomus grandis |
| HO | Curculio spp. |
| HO | Diabrotica balteata |
| HO | Leptinotarsa spp. |
| HO | Lissorhoptrus spp. |
| HO | Otiorhynchus spp. |
| HO | Aleurothrixus spp. |
| HO | Aleyrodes spp. |
| HO | Aonidiella spp. |
| HO | Aphididae spp. |
| HO | Aphis spp. |
| HO | Bemisia tabaci |
| HO | Empoasca spp. |
| HO | Mycus spp. |
| HO | Nephotettix spp. |
| HO | Nilaparvata spp. |
| HO | Pseudococcus spp. |
| HO | Psylla spp. |
| HO | Quadraspidiotus spp. |
| HO | Schizaphis spp. |
| HO | Trialeurodes spp. |
| HO | Lyriomyza spp. |
| HO | Oscinella spp. |
| HO | Phorbia spp. |
| HO | Frankliniella spp. |
| HO | Thrips spp. |
| HO | Scirtothrips aurantii |
| HO | Aceria spp. |
| HO | Acutus spp. |
| HO | Brevipalpus spp. |
| HO | Panonychus spp. |
| HO | Phyllocoptruta spp. |
| HO | Tetranychus spp. |
| HO | Heterodera spp. |
| HO | Meloidogyne spp. |

In the table, the following abbreviations were used:
active principle of the transgenic plant: AP
Photorhabdus luminescens: PL
Xenorhabdus nematophilus: XN
proteinase inhibitors: Plnh.
plant lectins PLec.
agglutinines: Aggl.
3-hydroxysteroid oxidase: HO
cholesterol oxidase: CO
chitinase: CH
glucanase: GL
stilbene synthase: SS

TABLE 3

| Principle | Tolerance to | Plant |
|---|---|---|
| ALS | sulphonylurea compounds etc.*** | cotton |
| ALS | sulphonylurea compounds etc.*** | rice |
| ALS | sulphonylurea compounds etc.*** | Brassica |
| ALS | sulphonylurea compounds etc.*** | potatoes |
| ALS | sulphonylurea compounds etc.*** | tomatoes |
| ALS | sulphonylurea compounds etc.*** | pumpkin |
| ALS | sulphonylurea compounds etc.*** | soya beans |
| ALS | sulphonylurea compounds etc.*** | maize |
| ALS | sulphonylurea compounds etc.*** | wheat |
| ALS | sulphonylurea compounds etc.*** | pome fruit |
| ALS | sulphonylurea compounds etc.*** | stone fruit |
| ALS | sulphonylurea compounds etc.*** | citrus fruit |
| ACCase | +++ | cotton |
| ACCase | +++ | rice |
| ACCase | +++ | Brassica |
| ACCase | +++ | potato |
| ACCase | +++ | tomatoes |
| ACCase | +++ | pumpkin |
| ACCase | +++ | soya beans |
| ACCase | +++ | maize |
| ACCase | +++ | wheat |
| ACCase | +++ | pome fruit |
| ACCase | +++ | stone fruit |
| ACCase | +++ | citrus fruit |
| HPPD | isoxaflutole, isoxachlortole, sulcotrione, | cotton |
| | mesotrione | |
| HPPD | isoxaflutole, isoxachlortole, sulcotrione, | rice |
| | mesotrione | |
| HPPD | isoxaflutole, isoxachlortole, sulcotrione, | Brassica |
| | mesotrione | |
| HPPD | isoxaflutole, isoxachlortole, sulcotrione, | potatoes |
| | mesotrione | |
| HPPD | isoxaflutole, isoxachlortole, sulcotrione, | tomatoes |
| | mesotrione | |
| HPPD | isoxaflutole, isoxachlortole, sulcotrione, | pumpkin |
| | mesotrione | |
| HPPD | isoxaflutole, isoxachlortole, sulcotrione, | soya beans |
| | mesotrione | |
| HPPD | isoxaflutole, isoxachlortole, sulcotrione, | maize |
| | mesotrione | |
| HPPD | isoxaflutole, isoxachlortole, sulcotrione, | wheat |
| | mesotrione | |
| HPPD | isoxaflutole, isoxachlortole, sulcotrione, | pome fruit |
| | mesotrione | |
| HPPD | isoxaflutole, isoxachlortole, sulcotrione, | stone fruit |
| | mesotrione | |
| HPPD | isoxaflutole, isoxachlortole, sulcotrione, | citrus fruit |
| | mesotrione | |
| nitrilase | bromoxynil, loxynil | cotton |
| nitrilase | bromoxynil, loxynil | rice |
| nitrilase | bromoxynil, loxynil | Brassica |
| nitrilase | bromoxynil, loxynil | potatoes |
| nitrilase | bromoxynil, loxynil | tomatoes |
| nitrilase | bromoxynil, loxynil | pumpkin |
| nitrilase | bromoxynil, loxynil | soya beans |
| nitrilase | bromoxynil, loxynil | maize |
| nitrilase | bromoxynil, loxynil | wheat |
| nitrilase | bromoxynil, loxynil | pome fruit |
| nitrilase | bromoxynil, loxynil | stone fruit |
| nitrilase | bromoxynil, loxynil | citrus fruit |
| IPS | chloroactanilides &&& | cotton |
| IPS | chloroactanilides &&& | rice |
| IPS | chloroactanilides &&& | Brassica |
| IPS | chloroactanilides &&& | potatoes |
| IPS | chloroactanilides &&& | tomatoes |
| IPS | chloroactanilides &&& | pumpkin |
| IPS | chloroactanilides &&& | soya beans |
| IPS | chloroactanilides &&& | maize |
| IPS | chloroactanilides &&& | wheat |
| IPS | chloroactanilides &&& | pome fruit |
| IPS | chloroactanilides &&& | stone fruit |
| IPS | chloroactanilides &&& | citrus fruit |
| HOM | 2,4-D, mecoprop-P | cotton |
| HOM | 2,4-D, mecoprop-P | rice |
| HOM | 2,4-D, mecoprop-P | Brassica |
| HOM | 2,4-D, mecoprop-P | potatoes |
| HOM | 2,4-D, mecoprop-P | tomatoes |
| HOM | 2,4-D, mecoprop-P | pumpkin |
| HOM | 2,4-D, mecoprop-P | soya beans |
| HOM | 2,4-D, mecoprop-P | maize |
| HOM | 2,4-D, mecoprop-P | wheat |
| HOM | 2,4-D, mecoprop-P | pome fruit |
| HOM | 2,4-D, mecoprop-P | stone fruit |
| HOM | 2,4-D, mecoprop-P | citrus fruit |
| PROTOX | Protox inhibitors /// | cotton |
| PROTOX | Protox inhibitors /// | rice |
| PROTOX | Protox inhibitors /// | Brassica |
| PROTOX | Protox inhibitors /// | potatoes |
| PROTOX | Protox inhibitors /// | tomatoes |
| PROTOX | Protox inhibitors /// | pumpkin |
| PROTOX | Protox inhibitors /// | soya beans |
| PROTOX | Protox inhibitors /// | maize |
| PROTOX | Protox inhibitors /// | wheat |
| PROTOX | Protox inhibitors /// | pome fruit |
| PROTOX | Protox inhibitors /// | stone fruit |
| PROTOX | Protox inhibitors /// | citrus fruit |
| EPSPS | glyphosate and/or sulphosate | cotton |
| EPSPS | glyphosate and/or sulphosate | rice |
| EPSPS | glyphosate and/or sulphosate | Brassica |
| EPSPS | glyphosate and/or sulphosate | potatoes |
| EPSPS | glyphosate and/or sulphosate | tomatoes |
| EPSPS | glyphosate and/or sulphosate | pumpkin |
| EPSPS | glyphosate and/or sulphosate | soya beans |
| EPSPS | glyphosate and/or sulphosate | maize |
| EPSPS | glyphosate and/or sulphosate | wheat |
| EPSPS | glyphosate and/or sulphosate | pome fruit |
| EPSPS | glyphosate and/or sulphosate | stone fruit |
| EPSPS | glyphosate and/or sulphosate | citrus fruit |
| GS | gluphosinate and/or bialaphos | cotton |
| GS | gluphosinate and/or bialaphos | rice |
| GS | gluphosinate and/or bialaphos | Brassica |
| GS | gluphosinate and/or bialaphos | potatoes |
| GS | gluphosinate and/or bialaphos | tomatoes |
| GS | gluphosinate and/or bialaphos | pumpkin |
| GS | gluphosinate and/or bialaphos | soya beans |
| GS | gluphosinate and/or bialaphos | maize |
| GS | gluphosinate and/or bialaphos | wheat |
| GS | gluphosinate and/or bialaphos | pome fruit |
| GS | gluphosinate and/or bialaphos | stone fruit |
| GS | gluphosinate and/or bialaphos | citrus fruit |

***included are sulphonylurea compounds, imidazolinones, triazolopyrimidines, dimethoxypyrimidines and N-acylsulphonamides: sulphonylurea compounds such as chlorsulfuron, chlorimuron, ethamethsulfuron, metsulfuron, primisulfuron, prosulfuron, triasulfuron, cinosulfuron, trifusulfuron, oxasulfuron, bensulfuron, tribenuron, ACC 322140, fluzasulfuron, ethoxysulfuron, fluzadsulfuron, nicosulfuron, rimsulfuron, thifensulfuron, pyrazosulfuron, clopyrasulfuron, NC 330, azimsulfuron, imazosulfuron, sulfosulfuron, amidosulfuron, flupyrsulfuron, CGA 362622 imidazolinones such as imazamethabenz, imazaquin, imazamethypyr, imazethapyr, imazapyr and imazamox; triazolopyrimidines such as DE 511, flumetsulam and chloransulam; dimethoxypyrimidines such as, for example, pyrithiobac, pyriminobac, bispyribac and pyribenzoxim.
+++ Tolerance to diclofop-methyl, fluazifop-P-butyl, haloxyfop-P-methyl, haloxyfop-P-ethyl, quizalafop-P-ethyl, clodinafop-propargyl, fenoxaprop-ethyl, tepraloxydim, alloxydim, sethoxydim, cycloxydim, cloproxydim, tralkoxydim, butoxydim, caloxydim, clefoxydim, clethodim.
&&& chloroacetanilides such as, for example, alachlor, acetochlor, dimethenamid
/// Protox inhibitors: for example diphenyl ethers such as, for example, acifluorfen, aclonifen, bifenox, chlornitrofen, ethoxyfen, fluoroglycofen, fomesafen, lactofen, oxyfluorfen; imides such as, for example, azafenidin, carfentrazone-ethyl, cinidon-ethyl, flumiclorac-pentyl, flumioxazin, fluthiacet-methyl, oxadiargyl, oxadiazon, pentoxazone, sulfentrazone, imides and other compounds such as, for example, flumipropyn, flupropacil, nipyraclofen and thidiazimin; and also fluazola and pyraflufen-ethyl.
Abbreviations:
acetyl-CoA carboxylase: ACCase
acetolactate synthase: ALS
hydroxyphenylpyruvate dioxygenase: HPPD
inhibition of protein synthesis: IPS
hormone imitation: HO
glutamine synthetase: GS
protoporphyrinogen oxidase: PROTOX
5-enolpyruvyl-3-phosphoshikimate synthase: EPSPS

TABLE 4
List of examples of transgenic plants having modified properties:

| Transgenic plants | Transgenically modified properties |
|---|---|
| Dianthus caryophyllus (carnation) | Longer-lasting as a result of reduced ethylene |
| Line 66 | accumulation owing to the expression of ACC |
| [Florigene Pty. Ltd.] | synthase; tolerant to sulphonylurea herbicides |
| Dianthus caryophyllus (carnation) | Modified flower colour; tolerant to sulphonyl- |
| Lines 4, 11, 15, 16 | urea herbicides |
| [Florigene Pty. Ltd.] | |
| Dianthus caryophyllus (carnation) | Modified flower colour; tolerant to sulphonyl- |
| Lines 959A, 988A, 1226A, 1351A, 1363A, | urea herbicides |
| 1400A | |
| [Florigene Pty. Ltd.] | |
| Brassica napus (Argentine oilseed rape) | Modified fatty acid content in the seeds |
| Lines 23-18-17, 23-198 | |
| [Monsanto Company] | |
| Zea mays L. (maize) | Elevated lysine content |
| Lines REN-ØØØ38-3 (LY038) | |
| [Monsanto Company] | |
| Zea mays L. (maize) | Elevated lysine content, corn borer resistant |
| Lines REN-ØØØ38-3, MON-ØØ81Ø-6 | |
| (MON-ØØ81Ø-6 x LY038) | |
| [Monsanto Company] | |
| Cucumis melo (melon) | Delayed maturity as a result of the expression of |
| Lines A, B | S-adenosylmethionine hydrolase |
| [Agritope Inc.] | |
| Carica papaya (papaya) | Resistant to the papaya ring spot virus (PRSV) |
| Lines 55-1/63-1 | |
| [Cornell University] | |
| Solanum tuberosum L. (potato) | Resistant to the Colorado beetle and the potato |
| Lines RBMT21-129, RBMT21-350, RBMT22- | leaf roll virus (PLRV) |
| 082 | |
| [Monsanto Company] | |
| Solanum tuberosum L. (potato) | Resistant to the Colorado beetle and the potato |
| Lines RBMT15-101, SEMT15-02, SEMT15-15 | virus Y (PVY) |
| [Monsanto Company] | |
| Glycine max L. (soyabean) | Modified fatty acid content in the seeds, in |
| Lines DD-Ø26ØØ5-3 (G94-1, G94-19, G168 | particular elevated oleic acid content |
| [DuPont Canada Agricultural Products] | |
| Glycine max L. (soyabean) | Modified fatty acid content in the seeds, in |
| Lines OT96-15 | particular reduced linolenic acid content |
| [Agriculture & Agri-Food Canada] | |
| Cucurbita pepo (pumpkin) | Resistant to viral infections, watermelon mosaic |
| Line ZW20 | virus (WMV) 2 and zucchini yellow mosaic |
| [Upjohn (USA); Seminis Vegetable Inc. | virus (ZYMV) |
| (Canada)] | |
| Cucurbita pepo (pumpkin) | Resistance to viral infections, cucumber mosaic |
| Line CZW-3 | virus (CMV), watermelon mosaic virus (WMV) |
| [Asgrow (USA); Seminis Vegetable Inc. | 2 and zucchini yellow mosaic virus (ZYMV) |
| (Canada)] | |
| Nicotiana tabacum L. (tobacco) | Reduced nicotine content |
| Line Vector 21-41 | |
| [Vector Tobacco] | |
| Lycopersicon esculentum (tomato) | Longer lasting as a result of reduced ethylene |
| Line 1345-4 | accumulation owing to the expression of ACC |
| [DNA Plant Technology] | synthase |
| Lycopersicon esculentum (tomato) | Delayed maturity as a result of the expression of |
| Line 35 1 N | S-adenosylmethionine hydrolase |
| [Agritope Inc.] | |
| Lycopersicon esculentum (tomato) | Delayed maturity as a result of the expression of |
| Line CGN-89322-3 (8338) | ACCd |
| [Monsanto Company] | |
| Lycopersicon esculentum (tomato) | Delayed softening as a result of a reduced |
| Lines B, Da, F | expression of polygalacturonase |
| [Zeneca Seeds] | |
| Lycopersicon esculentum (tomato) | Delayed softening as a result of a reduced |
| Line CGN-89564-2 (FLAVR SAVR) | expression of polygalacturonase |
| [Calgene Inc.] | |

TABLE 5

| No. | Line/trait | Commercial name | Plant |
|---|---|---|---|
| B-1 | ASR368 | | Agrostis stolonifera |
| | | | Creeping Bentgrass |
| B-2 | H7-1 | Roundup Ready Sugar | Beta vulgaris (Sugar Beet) |
| | | Beet | |
| B-3 | T120-7 | | Beta vulgaris (Sugar Beet) |
| B-4 | GTSB77 | | Beta vulgaris (Sugar Beet) |
| B-5 | 23-18-17, 23-198 | | Brassica napus (Argentine |
| | | | Canola) |
| B-6 | 45A37, 46A40 | | Brassica napus (Argentine |
| | | | Canola) |
| B-7 | 46A12, 46A16 | | Brassica napus (Argentine |
| | | | Canola) |
| B-8 | GT200 | | Brassica napus (Argentine |
| | | | Canola) |
| B-9 | GT73, RT73 | Roundup Ready ™ | Brassica napus (Argentine |
| | | canola | Canola) |
| B-10 | HCN10 | | Brassica napus (Argentine |
| | | | Canola) |
| B-11 | Topas 19/2 | InVigor ® Canola | Brassica napus (Argentine |
| | (HCN92) | | Canola) |
| B-12 | MS1, RF1 =>PGS1 | | Brassica napus (Argentine |
| | | | Canola) |
| B-13 | MS1, RF2 =>PGS2 | | Brassica napus (Argentine |
| | | | Canola) |
| B-14 | MS8 × RF3 | InVigor ® Canola | Brassica napus (Argentine |
| | | | Canola) |
| B-15 | NS738, NS1471, | | Brassica napus (Argentine |
| | NS1473 | | Canola) |
| B-16 | OXY-235 | | Brassica napus (Argentine |
| | | | Canola) |
| B-17 | MS8 | InVigor ® Canola | Brassica napus (Argentine |
| | | | Canola) |
| B-18 | PHY14, PHY35 | | Brassica napus (Argentine |
| | | | Canola) |
| B-19 | PHY36 | | Brassica napus (Argentine |
| | | | Canola) |
| B-20 | RF1, (B93-101) | InVigor ® Canola | Brassica napus (Argentine |
| | | | Canola) |
| B-21 | RF2, (B94-101) | | Brassica napus (Argentine |
| | | | Canola) |
| B-22 | RF3, ACS- | InVigor ® Canola | Brassica napus (Argentine |
| | BNØØ3-6 | | Canola) |
| B-23 | MS1 (B91-4) | InVigor ® Canola | Brassica napus (Argentine |
| | | | Canola) |
| B-24 | T45 (HCN28) | InVigor ® Canola | Brassica napus (Argentine |
| | | | Canola) |
| B-25 | HCR-1 | | Brassica rapa (Polish |
| | | | Canola) |
| B-26 | ZSR500/502 | | Brassica rapa (Polish |
| | | | Canola) |
| B-27 | 55-1/63-1 | | Carica papaya (Papaya) |
| B-28 | RM3-3, RM3-4, | | Cichorium intybus (Chicory) |
| | RM3-6 | | |
| B-29 | A, B | | Cucumis melo (Melon) |
| B-30 | CZW-3 | | Cucurbita pepo (Squash) |
| B-31 | ZW20 | | Cucurbita pepo (Squash) |
| B-32 | 66 | | Dianthus |
| | | | caryophyllus (Carnation) |
| B-33 | 4, 11, 15, 16 | | Dianthus |
| | | | caryophyllus (Carnation) |
| B-34 | 11363 | Moonshadow | Dianthus |
| | | | caryophyllus (Carnation) |
| B-35 | 959A, 988A, | | Dianthus |
| | 1226A, 1351A, | | caryophyllus (Carnation) |
| | 1363A, 1400A | | |
| B-36 | 123.2. (40619) | Moonshade | Dianthus |
| | | | caryophyllus (Carnation) |
| B-37 | 123.8.8 (40685) | Moonvista | Dianthus |
| | | | caryophyllus (Carnation) |
| B-38 | 11 (7442) | Moondust | Dianthus |
| | | | caryophyllus (Carnation) |
| B-39 | A2704-12, A2704- | | Glycine max L. (Soybean) |
| | 21, A5547-35 | | |
| B-40 | A5547-127 | LibertyLink ® Soybean | Glycine max L. (Soybean) |
| B-41 | G94-1, G94-19, | | Glycine max L. (Soybean) |
| | G168 | | |
| B-42 | GTS 40-3-2 | Roundup Ready ™ | Glycine max L. (Soybean) |
| | | soybeans | |
| B-43 | GU262 | | Glycine max L. (Soybean) |
| B-44 | MON89788 | Roundup | Glycine max L. (Soybean) |
| | | RReady2Yield ™ | |
| | | soybean | |
| B-45 | OT96-15 | | Glycine max L. (Soybean) |
| B-46 | W62, W98 | | Glycine max L. (Soybean) |
| B-47 | 15985 | Bollgard II cotton | Gossypium hirsutum L. |
| | | | (Cotton) |
| B-48 | 19-51A | | Gossypium hirsutum L. |
| | | | (Cotton) |
| B-49 | 281-24-236 | | Gossypium hirsutum L. |
| | | | (Cotton) |
| B-50 | 3006-210-23 | WideStrike ™ | Gossypium hirsutum L. |
| | | | (Cotton) |
| B-51 | 31807/31808 | | Gossypium hirsutum L. |
| | | | (Cotton) |
| B-52 | BXN | | Gossypium hirsutum L. |
| | | | (Cotton) |
| B-53 | COT102 | | Gossypium hirsutum L. |
| | | | (Cotton) |
| B-54 | DAS-21Ø23-5 × | | Gossypium hirsutum L. |
| | DAS-24236-5 | | (Cotton) |
| B-55 | DAS-21Ø23-5 × | | Gossypium hirsutum L. |
| | DAS-24236-5 × | | (Cotton) |
| | MON88913 | | |
| B-56 | DAS-21Ø23-5 × | | Gossypium hirsutum L. |
| | DAS-24236-5 × | | (Cotton) |
| | MON-Ø1445-2 | | |
| B-57 | LLCotton25 | | Gossypium hirsutum L. |
| | | | (Cotton) |
| B-58 | LLCotton25 × | | Gossypium hirsutum L. |
| | MON15985 | | (Cotton) |
| B-59 | MON1445/1698 | Roundup Ready ™ | Gossypium hirsutum L. |
| | | cotton | (Cotton) |
| B-60 | MON15985 × | | Gossypium hirsutum L. |
| | MON88913 | | (Cotton) |
| B-61 | MON-15985-7 × | | Gossypium hirsutum L. |
| | MON-Ø1445-2 | | (Cotton) |
| B-62 | MON531/757/1076 | Bollgard ™ (Ingard ®) | Gossypium hirsutum L. |
| | | | (Cotton) |
| B-63 | MON88913 | Roundup Ready Flex | Gossypium hirsutum L. |
| | | Cotton | (Cotton) |
| B-64 | MON-ØØ531-6 × | | Gossypium hirsutum L. |
| | MON-Ø1445-2 | | (Cotton) |
| B-65 | T304-40 | | Gossypium hirsutum L. |
| | | | (Cotton) |
| B-66 | GHB714 | | Gossypium hirsutum L. |
| | | | (Cotton) |
| B-67 | GHB119 | | Gossypium hirsutum L. |
| | | | (Cotton) |
| B-68 | T303-3 | | Gossypium hirsutum L. |
| | | | (Cotton) |
| B-69 | GHB614 | | Gossypium hirsutum L. |
| | | | (Cotton) |
| B-70 | X81359 | | Helianthus |
| | | | annuus (Sunflower) |
| B-71 | RH44 | | Lens culinaris (Lentil) |
| B-72 | FP967 | | Linum usitatissimum L. |
| | | | (Flax, Linseed) |
| B-73 | 5345 | | Lycopersicon |
| | | | esculentum (Tomato) |
| B-74 | 8338 | | Lycopersicon |
| | | | esculentum (Tomato) |
| B-75 | 1345-4 | | Lycopersicon |
| | | | esculentum (Tomato) |
| B-76 | 35 1 N | | Lycopersicon |
| | | | esculentum (Tomato) |
| B-77 | B, Da, F | | Lycopersicon |
| | | | esculentum (Tomato) |
| B-78 | FLAVR SAVR | FLAVR SAVR | Lycopersicon |
| | | | esculentum (Tomato) |
| B-79 | J101, J163 | Roundup Ready | Medicago sativa (Alfalfa) |
| | | Alfalfa | |
| B-80 | C/F/93/08-02 | | Nicotiana tabacum L. |
| | | | (Tobacco) |
| B-81 | Vector 21-41 | | Nicotiana tabacum L. |
| | | | (Tobacco) |
| B-82 | CL121, CL141, | | Oryza sativa (Rice) |
| | CFX51 | | |
| B-83 | IMINTA-1, | Clearfield ™ | Oryza sativa (Rice) |
| | IMINTA-4 | | |
| B-84 | LLRICE06, | LibertyLink ® Rice | Oryza sativa (Rice) |
| | LLRICE62 | | |
| B-85 | LLRICE601 | | Oryza sativa (Rice) |
| B-86 | PWC16 | | Oryza sativa (Rice) |
| B-87 | ATBT04-6, | NewLeaf Atlantic | Solanum tuberosum L. |
| | ATBT04-27, | | (Potato) |
| | ATBT04-30, | | |
| | ATBT04-31, | | |
| | ATBT04-36, | | |
| | SPBT02-5, | | |
| | SPBT02-7 | | |
| B-88 | BT6, BT10, BT12, | NewLeaf Russet | Solanum tuberosum L. |
| | BT16, BT17, | Burbank | (Potato) |
| | BT18, BT23 | | |
| B-89 | RBMT15-101, | | Solanum tuberosum L. |
| | SEMT15-02, | | (Potato) |
| | SEMT15-15 | | |
| B-90 | RBMT21-129, | | Solanum tuberosum L. |
| | RBMT21-350, | | (Potato) |
| | RBMT22-082 | | |
| B-91 | AM02-1003, | | Solanum tuberosum L. |
| | AM01-1005, | | (Potato) |
| | AM02-1012, | | |
| | AM02-1017, | | |
| | AM99-1089 and | | |
| | AM99-2003 | | |
| B-92 | EH92-527-1 | Amflora | Solanum tuberosum L. |
| | | | (Potato) |
| B-93 | AP205CL | | Triticum aestivum (Wheat) |
| B-94 | AP602CL | | Triticum aestivum (Wheat) |
| B-95 | BW255-2, BW238-3 | Clearfield ™ | Triticum aestivum (Wheat) |
| B-96 | MON71800 | | Triticum aestivum (Wheat) |
| B-97 | SWP965001 | | Triticum aestivum (Wheat) |
| B-98 | DW2, DW6, | Clearfield ™ | Triticum aestivum (Wheat) |
| | DW12 | | |
| B-99 | BW7 | Clearfield ™ | Triticum aestivum (Wheat) |
| B-100 | Teal 11A | | Triticum aestivum (Wheat) |
| B-101 | 176 | Knockout ™, | Zea mays L. (Maize) |
| | | NautureGard ™ | |
| B-102 | 3751IR | | Zea mays L. (Maize) |
| B-103 | 676, 678, 680 | LibertyLink ® Male | Zea mays L. (Maize) |
| | | Sterile | |
| B-104 | ACS-ZMØØ3-2 × | | Zea mays L. (Maize) |
| | MON-ØØ81Ø-6 | | |
| B-105 | B16 (DLL25) | | Zea mays L. (Maize) |
| B-106 | BT11 (X4334CBR, | BiteGard ® | Zea mays L. (Maize) |
| | X4734CBR) | | |
| B-107 | CBH-351 | StarLink ® | Zea mays L. (Maize) |
| B-108 | DAS-06275-8 | | Zea mays L. (Maize) |
| B-109 | DAS-59122-7 | Herculex RW | Zea mays L. (Maize) |
| | | Rootworm Protection | |
| | | Maise | |
| B-110 | DAS-59122-7 × | | Zea mays L. (Maize) |
| | NK603 | | |
| B-111 | DAS-59122-7 × | | Zea mays L. (Maize) |
| | TC1507 × NK603 | | |
| B-112 | DAS-Ø15Ø7-1 × | | Zea mays L. (Maize) |
| | MON-ØØ6Ø3-6 | | |
| B-113 | DBT418 | Bt-XTRA ® | Zea mays L. (Maize) |
| B-114 | DK404SR | | Zea mays L. (Maize) |
| B-115 | EXP1910IT | | Zea mays L. (Maize) |
| B-116 | GA21 | Roundup Ready ® | Zea mays L. (Maize) |
| B-117 | IT | | Zea mays L. (Maize) |
| B-118 | LY038 | Mavera ™ High Value | Zea mays L. (Maize) |
| | | Corn with Lysine | |
| B-119 | MIR604 | Agrisure RW | Zea mays L. (Maize) |
| | | Rootworm-Protected | |
| | | Corn | |
| B-120 | MON80100 | | Zea mays L. (Maize) |
| B-121 | MON802 | Roundup Ready ® | Zea mays L. (Maize) |
| B-122 | MON809 | | Zea mays L. (Maize) |
| B-123 | MON810 | YieldGard ® | Zea mays L. (Maize) |
| B-124 | MON810 × | | Zea mays L. (Maize) |
| | MON88017 | | |
| B-125 | MON832 | | Zea mays L. (Maize) |
| B-126 | MON863 | YieldGard ® | Zea mays L. (Maize) |
| | | Rootworm | |
| B-127 | MON88017 | | Zea mays L. (Maize) |
| B-128 | MON-ØØ6Ø3-6 × | | Zea mays L. (Maize) |
| | MON-ØØ81Ø-6 | | |
| B-129 | MON-ØØ81Ø-6 × | | Zea mays L. (Maize) |
| | LY038 | | |
| B-130 | MON-ØØ863-5 × | | Zea mays L. (Maize) |
| | MON-ØØ6Ø3-6 | | |
| B-131 | MON-ØØ863-5 × | YieldGard ® Plus | Zea mays L. (Maize) |
| | MON-ØØ81Ø-6 | | |
| B-132 | MON-ØØ863-5 × | YieldGard ® Plus, | Zea mays L. (Maize) |
| | MON-ØØ81Ø-6 × | Roundup Ready ® | |
| | MON-ØØ6Ø3-6 | | |
| B-133 | MON-ØØØ21-9 × | | Zea mays L. (Maize) |
| | MON-ØØ81Ø-6 | | |
| B-134 | MS3 | | Zea mays L. (Maize) |
| B-135 | MS6 | LibertyLink ® Male | Zea mays L. (Maize) |
| | | Sterile | |
| B-136 | NK603 | Roundup Ready ® corn | Zea mays L. (Maize) |
| B-137 | SYN-BTØ11-1 × | | Zea mays L. (Maize) |
| | MON-ØØØ21-9 | | |
| B-138 | T14, T25 | LibertyLink ™ | Zea mays L. (Maize) |
| B-139 | TC1507 | Herculex I ® | Zea mays L. (Maize) |
| B-140 | TC1507 × DAS- | | Zea mays L. (Maize) |
| | 59122-7 | | |
| B-141 | SYTGA21 | | Zea mays L. (Maize) |
| B-142 | SYTGA21 + Btl1 | | Zea mays L. (Maize) |
| B-143 | MON810 + | | Zea mays L. (Maize) |
| | SYTGA21 | | |
| B-144 | MON89034 | | Zea mays L. (Maize) |
| B-145 | MON 89034 × | | Zea mays L. (Maize) |
| | MON 88017 | | |
| B-146 | MON 89034 × | | Zea mays L. (Maize) |
| | NK603 | | |
| B-147 | DP-Ø9814Ø-6 | | Zea mays L. (Maize) |
| B-148 | 3243M | | Zea mays L. (Maize) |
| B-149 | DP 444 BG/RR | Bollgard/RoundupReady, | Gossypium hirsutum |
| | | from US | L. (Cotton) |
| | | 2003213029-A1 | |
| B-150 | VSN-BTCRW | Bt-toxin corn root | Zea mays L. (Maize) |
| | | worm | |
| B-151 | HCL201CRW2RR × | Bt-toxin corn root | Zea mays L. (Maize) |
| | LH324 | worm | |
| B-152 | LH324 | from U.S. Pat. No. 7,223,908 B1 | Zea mays L. (Maize) |
| B-153 | VSN-RR Bt | RoundupReady Bt- | Zea mays L. (Maize) |
| | | toxin | |
| B-154 | FR1064LL × | Ref: Gerdes, J. T., | Zea mays L. (Maize) |
| | FR2108 | Behr, C. F., Coors, J. G., | |
| | | and Tracy, W. F. | |
| | | 1993. Compilation | |
| | | of North American | |
| | | Maize Breeding | |
| | | Germplasm. W. F. Tracy, | |
| | | J. G. Coors, | |
| | | and J. L. Geadelmann, | |
| | | eds. | |
| | | Crop Science Society | |
| | | of America, | |
| | | Madison, WI and U.S. Pat. No. | |
| | | 6,407,320 B1 | |
| B-155 | VSN-Bt | Bt-toxin | Zea mays L. (Maize) |

| No. | Company | Transgenically modified properties |
|---|---|---|
| B-1 | Scotts Seeds | Glyphosate tolerance derived by inserting a |
| | | modified 5-enolpyruvylshikimate-3-phosphate |
| | | synthase (EPSPS) encoding gene from |
| | | Agrobacterium tumefaciens. |
| B-2 | Monsanto Company | Glyphosate herbicide tolerant sugar beet produced |
| | | by inserting a gene encoding the enzyme 5- |
| | | enolypyruvylshikimate-3-phosphate synthase |
| | | (EPSPS) from the CP4 strain of Agrobacterium |
| | | tumefaciens. |
| B-3 | Bayer CropScience | Introduction of the PPT-acetyltransferase (PAT) |
| | (Aventis | encoding gene from Streptomyces |
| | CropScience(AgrEvo)) | viridochromogenes, an aerobic soil bacteria. PPT |
| | | normally acts to inhibit glutamine synthetase, |
| | | causing a fatal accumulation of ammonia. |
| | | Acetylated PPT is inactive. |
| B-4 | Novartis Seeds; Monsanto | Glyphosate herbicide tolerant sugar beet produced |
| | Company | by inserting a gene encoding the enzyme 5- |
| | | enolypyruvylshikimate-3-phosphate synthase |
| | | (EPSPS) from the CP4 strain of Agrobacterium |
| | | tumefaciens. |
| B-5 | Monsanto Company | High laurate (12:0) and myristate (14:0) canola |
| | (formerly Calgene) | produced by inserting a thioesterase encoding |
| | | gene from the California bay laurel (Umbellularia |
| | | californica). |
| B-6 | Pioneer Hi-Bred | High oleic acid and low linolenic acid canola |
| | International Inc. | produced through a combination of chemical |
| | | mutagenesis to select for a fatty acid desaturase |
| | | mutant with elevated oleic acid, and traditional |
| | | back-crossing to introduce the low linolenic acid |
| | | trait. |
| B-7 | Pioneer Hi-Bred | Combination of chemical mutagenesis, to achieve |
| | International Inc. | the high oleic acid trait, and traditional breeding |
| | | with registered canola varieties. |
| B-8 | Monsanto Company | Glyphosate herbicide tolerant canola produced by |
| | | inserting genes encoding the enzymes 5- |
| | | enolypyruvylshikimate-3-phosphate synthase |
| | | (EPSPS) from the CP4 strain of Agrobacterium |
| | | tumefaciens and glyphosate oxidase from |
| | | Ochrobactrum anthropi. |
| B-9 | Monsanto Company | Glyphosate herbicide tolerant canola produced by |
| | | inserting genes encoding the enzymes 5- |
| | | enolypyruvylshikimate-3-phosphate synthase |
| | | (EPSPS) from the CP4 strain of Agrobacterium |
| | | tumefaciens and glyphosate oxidase from |
| | | Ochrobactrum anthropi. |
| B-10 | Aventis CropScience | Introduction of the PPT-acetyltransferase (PAT) |
| | | encoding gene from Streptomyces |
| | | viridochromogenes, an aerobic soil bacteria. PPT |
| | | normally acts to inhibit glutamine synthetase, |
| | | causing a fatal accumulation of ammonia. |
| | | Acetylated PPT is inactive. |
| B-11 | Bayer CropScience | Introduction of the PPT-acetyltransferase (PAT) |
| | (Aventis | encoding gene from Streptomyces |
| | CropScience(AgrEvo)) | viridochromogenes, an aerobic soil bacteria. PPT |
| | | normally acts to inhibit glutamine synthetase, |
| | | causing a fatal accumulation of ammonia. |
| | | Acetylated PPT is inactive. |
| B-12 | Aventis CropScience | Male-sterility, fertility restoration, pollination |
| | (formerly Plant Genetic | control system displaying glufosinate herbicide |
| | Systems) | tolerance. MS lines contained the barnase gene |
| | | from Bacillus amyloliquefaciens, RF lines |
| | | contained the barstar gene from the same bacteria, |
| | | and both lines contained the phosphinothricin N- |
| | | acetyltransferase (PAT) encoding gene from |
| | | Streptomyces hygroscopicus. |
| B-13 | Aventis CropScience | Male-sterility, fertility restoration, pollination |
| | (formerly Plant Genetic | control system displaying glufosinate herbicide |
| | Systems) | tolerance. MS lines contained the barnase gene |
| | | from Bacillus amyloliquefaciens, RF lines |
| | | contained the barstar gene from the same bacteria, |
| | | and both lines contained the phosphinothricin N- |
| | | acetyltransferase (PAT) encoding gene from |
| | | Streptomyces hygroscopicus. |
| B-14 | Bayer CropScience | Male-sterility, fertility restoration, pollination |
| | (Aventis | control system displaying glufosinate herbicide |
| | CropScience(AgrEvo)) | tolerance. MS lines contained the barnase gene |
| | | from Bacillus amyloliquefaciens, RF lines |
| | | contained the barstar gene from the same bacteria, |
| | | and both lines contained the phosphinothricin N- |
| | | acetyltransferase (PAT) encoding gene from |
| | | Streptomyces hygroscopicus. |
| B-15 | Pioneer Hi-Bred | Selection of somaclonal variants with altered |
| | International Inc. | acetolactate synthase (ALS) enzymes, following |
| | | chemical mutagenesis. Two lines (P1, P2) were |
| | | initially selected with modifications at different |
| | | unlinked loci. NS738 contains the P2 mutation |
| | | only. |
| B-16 | Aventis CropScience | Tolerance to the herbicides bromoxynil and |
| | (formerly Rhone Poulenc | ioxynil by incorporation of the nitrilase gene |
| | Inc.) | (oxy) from Klebsiella pneumoniae. |
| B-17 | Bayer CropScience | Traits: Glufosinate tolerance, Male sterility |
| | | Genes: bar, barnase |
| B-18 | Aventis CropScience | Male sterility was via insertion of the barnase |
| | (formerly Plant Genetic | ribonuclease gene from Bacillus |
| | Systems) | amyloliquefaciens; fertility restoration by |
| | | insertion of the barstar RNase inhibitor; PPT |
| | | resistance was via PPT-acetyltransferase (PAT) |
| | | from Streptomyces hygroscopicus. |
| B-19 | Aventis CropScience | Male sterility was via insertion of the barnase |
| | (formerly Plant Genetic | ribonuclease gene from Bacillus |
| | Systems) | amyloliquefaciens; fertility restoration by |
| | | insertion of the barstar RNase inhibitor; PPT |
| | | resistance was via PPT-acetyltransferase (PAT) |
| | | from Streptomyces hygroscopicus. |
| B-20 | Bayer CropScience | Genes: bar, barstar, neomycin phosphotransferase |
| | | II (npt II); Traits: Fertility restoration, Glufosinate |
| | | tolerance, Kanamycin resistance |
| B-21 | Bayer CropScience | Genes: bar, barstar, neomycin phosphotransferase |
| | | II (npt II); Traits: Fertility restoration, Glufosinate |
| | | tolerance, Kanamycin resistance |
| B-22 | Bayer CropScience | Traits: Fertility restoration, Glufosinate tolerance; |
| | | Genes bar, barstar |
| B-23 | Bayer CropScience | Traits: Glufosinate tolerance, Kanamycin |
| | | resistance, Male sterility; Genes: |
| | | bar, barnase, neomycin phosphotransferase II (npt |
| | | II) |
| B-24 | Bayer CropScience | Introduction of the PPT-acetyltransferase (PAT) |
| | (Aventis | encoding gene from Streptomyces |
| | CropScience(AgrEvo)) | viridochromogenes, an aerobic soil bacteria. PPT |
| | | normally acts to inhibit glutamine synthetase, |
| | | causing a fatal accumulation of ammonia. |
| | | Acetylated PPT is inactive. |
| B-25 | Bayer CropScience | Introduction of the glufosinate ammonium |
| | (Aventis | herbicide tolerance trait from transgenic B. napus |
| | CropScience(AgrEvo)) | line T45. This trait is mediated by the |
| | | phosphinothricin acetyltransferase (PAT) |
| | | encoding gene from S. viridochromogenes. |
| B-26 | Monsanto Company | Introduction of a modified 5-enol- |
| | | pyruvylshikimate-3-phosphate synthase (EPSPS) |
| | | and a gene from Achromobacter sp that degrades |
| | | glyphosate by conversion to |
| | | aminomethylphosphonic acid (AMPA) and |
| | | glyoxylate by interspecific crossing with GT73. |
| B-27 | Cornell University | Papaya ringspot virus (PRSV) resistant papaya |
| | | produced by inserting the coat protein (CP) |
| | | encoding sequences from this plant potyvirus. |
| B-28 | Bejo Zaden BV | Male sterility was via insertion of the barnase |
| | | ribonuclease gene from Bacillus |
| | | amyloliquefaciens; PPT resistance was via the bar |
| | | gene from S. hygroscopicus, which encodes the |
| | | PAT enzyme. |
| B-29 | Agritope Inc. | Reduced accumulation of S-adenosylmethionine |
| | | (SAM), and consequently reduced ethylene |
| | | synthesis, by introduction of the gene encoding S- |
| | | adenosylmethionine hydrolase. |
| B-30 | Asgrow (USA); Seminis | Cucumber mosiac virus (CMV), zucchini yellows |
| | Vegetable Inc. (Canada) | mosaic (ZYMV) and watermelon mosaic virus |
| | | (WMV) 2 resistant squash (Curcurbita pepo) |
| | | produced by inserting the coat protein (CP) |
| | | encoding sequences from each of these plant |
| | | viruses into the host genome. |
| B-31 | Upjohn (USA); Seminis | Zucchini yellows mosaic (ZYMV) and |
| | Vegetable Inc. (Canada) | watermelon mosaic virus (WMV) 2 resistant |
| | | squash (Curcurbita pepo) produced by inserting |
| | | the coat protein (CP) encoding sequences from |
| | | each of these plant potyviruses into the host |
| | | genome. |
| B-32 | Florigene Pty Ltd. | Delayed senescence and sulfonylurea herbicide |
| | | tolerant carnations produced by inserting a |
| | | truncated copy of the carnation |
| | | aminocyclopropane cyclase (ACC) synthase |
| | | encoding gene in order to suppress expression of |
| | | the endogenous unmodified gene, which is |
| | | required for normal ethylene biosynthesis. |
| | | Tolerance to sulfonyl urea herbicides was via the |
| | | introduction of a chlorsulfuron tolerant version of |
| | | the acetolactate synthase (ALS) encoding gene |
| | | from tobacco. |
| B-33 | Florigene Pty Ltd. | Modified colour and sulfonylurea herbicide |
| | | tolerant carnations produced by inserting two |
| | | anthocyanin biosynthetic genes whose expression |
| | | results in a violet/mauve colouration. Tolerance to |
| | | sulfonyl urea herbicides was via the introduction |
| | | of a chlorsulfuron tolerant version of the |
| | | acetolactate synthase (ALS) encoding gene from |
| | | tobacco. |
| B-34 | Florigene Pty Ltd. | Traits: Coloration; Genes als, dihydroflavonol |
| | | reductase (dfr), flavonoid 3′,5′hydroxylase (F3′5′H) |
| B-35 | Florigene Pty Ltd. | Introduction of two anthocyanin biosynthetic |
| | | genes to result in a violet/mauve colouration; |
| | | Introduction of a variant form of acetolactate |
| | | synthase (ALS). |
| B-36 | Florigene Pty Ltd. | Traits: Coloration; Genes als, dihydroflavonol |
| | | reductase (dfr), flavonoid 3′,5′hydroxylase (F3′5′H) |
| B-37 | Florigene Pty Ltd. | |
| B-38 | Florigene Pty Ltd. | |
| B-39 | Aventis CropScience | Glufosinate ammonium herbicide tolerant |
| | | soybean produced by inserting a modified |
| | | phosphinothricin acetyltransferase (PAT) |
| | | encoding gene from the soil bacterium |
| | | Streptomyces viridochromogenes. |
| B-40 | Bayer CropScience | Glufosinate ammonium herbicide tolerant |
| | (Aventis | soybean produced by inserting a modified |
| | CropScience(AgrEvo)) | phosphinothricin acetyltransferase (PAT) |
| | | encoding gene from the soil bacterium |
| | | Streptomyces viridochromogenes. |
| B-41 | DuPont Canada | High oleic acid soybean produced by inserting a |
| | Agricultural Products | second copy of the fatty acid desaturase |
| | | (GmFad2-1) encoding gene from soybean, which |
| | | resulted in “silencing” of the endogenous host |
| | | gene. |
| B-42 | Monsanto Company | Glyphosate tolerant soybean variety produced by |
| | | inserting a modified 5-enolpyruvylshikimate-3- |
| | | phosphate synthase (EPSPS) encoding gene from |
| | | the soil bacterium Agrobacterium tumefaciens. |
| B-43 | Bayer CropScience | Glufosinate ammonium herbicide tolerant |
| | (Aventis | soybean produced by inserting a modified |
| | CropScience(AgrEvo)) | phosphinothricin acetyltransferase (PAT) |
| | | encoding gene from the soil bacterium |
| | | Streptomyces viridochromogenes. |
| B-44 | Monsanto Company | Glyphosate-tolerant soybean produced by |
| | | inserting a modified 5-enolpyruvylshikimate-3- |
| | | phosphate synthase (EPSPS) encoding aroA |
| | | (epsps) gene from Agrobacterium tumefaciens |
| | | CP4. |
| B-45 | Agriculture & Agri-Food | Low linolenic acid soybean produced through |
| | Canada | traditional cross-breeding to incorporate the novel |
| | | trait from a naturally occurring fanl gene mutant |
| | | that was selected for low linolenic acid. |
| B-46 | Bayer CropScience | Glufosinate ammonium herbicide tolerant |
| | (Aventis | soybean produced by inserting a modified |
| | CropScience(AgrEvo)) | phosphinothricin acetyltransferase (PAT) |
| | | encoding gene from the soil bacterium |
| | | Streptomyces hygroscopicus. |
| B-47 | Monsanto Company | Insect resistant cotton derived by transformation |
| | | of the DP50B parent variety, which contained |
| | | event 531 (expressing Cry1Ac protein), with |
| | | purified plasmid DNA containing the cry2Ab |
| | | gene from B. thuringiensis subsp. kurstaki. |
| B-48 | DuPont Canada | Introduction of a variant form of acetolactate |
| | Agricultural Products | synthase (ALS). |
| B-49 | DOW AgroSciences LLC | Insect-resistant cotton produced by inserting the |
| | | cry1F gene from Bacillus thuringiensis var. |
| | | aizawai. The PAT encoding gene from |
| | | Streptomyces viridochromogenes was introduced |
| | | as a selectable marker. |
| B-50 | DOW AgroSciences LLC | Insect-resistant cotton produced by inserting the |
| | | cry1Ac gene from Bacillus thuringiensis subsp. |
| | | kurstaki. The PAT encoding gene from |
| | | Streptomyces viridochromogenes was introduced |
| | | as a selectable marker. |
| B-51 | Calgene Inc. | Insect-resistant and bromoxynil herbicide tolerant |
| | | cotton produced by inserting the cry1Ac gene |
| | | from Bacillus thuringiensis and a nitrilase |
| | | encoding gene from Klebsiella pneumoniae. |
| B-52 | Calgene Inc. | Bromoxynil herbicide tolerant cotton produced by |
| | | inserting a nitrilase encoding gene from |
| | | Klebsiella pneumoniae. |
| B-53 | Syngenta Seeds, Inc. | Insect-resistant cotton produced by inserting the |
| | | vip3A(a) gene from Bacillus thuringiensis AB88. |
| | | The APH4 encoding gene from E. coli was |
| | | introduced as a selectable marker. |
| B-54 | DOW AgroSciences LLC | WideStrike ™, a stacked insect-resistant cotton |
| | | derived from conventional cross-breeding of |
| | | parental lines 3006-210-23 (OECD identifier: |
| | | DAS-21Ø23-5) and 281-24-236 (OECD |
| | | identifier: DAS-24236-5). |
| B-55 | DOW AgroSciences LLC | Stacked insect-resistant and glyphosate-tolerant |
| | and Pioneer Hi-Bred | cotton derived from conventional cross-breeding |
| | International Inc. | of WideStrike cotton (OECD identifier: DAS- |
| | | 21Ø23-5 × DAS-24236-5) with MON88913, |
| | | known as RoundupReady Flex (OECD identifier: |
| | | MON-88913-8). |
| B-56 | DOW AgroSciences LLC | WideStrike ™/Roundup Ready ® cotton, a stacked |
| | | insect-resistant and glyphosate-tolerant cotton |
| | | derived from conventional cross-breeding of |
| | | WideStrike cotton (OECD identifier: DAS- |
| | | 21Ø23-5 × DAS-24236-5) with MON1445 |
| | | (OECD identifier: MON-Ø1445-2). |
| B-57 | Bayer CropScience | Glufosinate ammonium herbicide tolerant cotton |
| | (Aventis | produced by inserting a modified |
| | CropScience(AgrEvo)) | phosphinothricin acetyltransferase (PAT) |
| | | encoding gene from the soil bacterium |
| | | Streptomyces hygroscopicus. |
| B-58 | Bayer CropScience | Stacked herbicide tolerant and insect resistant |
| | (Aventis | cotton combining tolerance to glufosinate |
| | CropScience(AgrEvo)) | ammonium herbicide from LLCotton25 (OECD |
| | | identifier: ACS-GHØØ1-3) with resistance to |
| | | insects from MON15985 (OECD identifier: |
| | | MON-15985-7) |
| B-59 | Monsanto Company | Glyphosate herbicide tolerant cotton produced by |
| | | inserting a naturally glyphosate tolerant form of |
| | | the enzyme 5-enolpyruvyl shikimate-3-phosphate |
| | | synthase (EPSPS) from A. tumefaciens strain |
| | | CP4. |
| B-60 | Monsanto Company | Stacked insect resistant and glyphosate tolerant |
| | | cotton produced by conventional cross-breeding |
| | | of the parental lines MON88913 (OECD |
| | | identifier: MON-88913-8) and 15985 (OECD |
| | | identifier: MON-15985-7). Glyphosate tolerance |
| | | is derived from MON88913 which contains two |
| | | genes encoding the enzyme 5- |
| | | enolypyruvylshikimate-3-phosphate synthase |
| | | (EPSPS) from the CP4 strain of Agrobacterium |
| | | tumefaciens. Insect resistance is derived |
| | | MON15985 which was produced by |
| | | transformation of the DP50B parent variety, |
| | | which contained event 531 (expressing Cry1Ac |
| | | protein), with purified plasmid DNA containing |
| | | the cry2Ab gene from B. thuringiensis subsp. |
| | | kurstaki. |
| B-61 | Monsanto Company | Stacked insect resistant and herbicide tolerant |
| | | cotton derived from conventional cross-breeding |
| | | of the parental lines 15985 (OECD identifier: |
| | | MON-15985-7) and MON1445 (OECD identifier: |
| | | MON-Ø1445-2). |
| B-62 | Monsanto Company | Insect-resistant cotton produced by inserting the |
| | | cry1Ac gene from Bacillus thuringiensis subsp. |
| | | kurstaki HD-73 (B.t.k.). |
| B-63 | Monsanto Company | Glyphosate herbicide tolerant cotton produced by |
| | | inserting two genes encoding the enzyme 5- |
| | | enolypyruvylshikimate-3-phosphate synthase |
| | | (EPSPS) from the CP4 strain of Agrobacterium |
| | | tumefaciens. |
| B-64 | Monsanto Company | Stacked insect resistant and herbicide tolerant |
| | | cotton derived from conventional cross-breeding |
| | | of the parental lines MON531 (OECD identifier: |
| | | MON-ØØ531-6) and MON1445 (OECD |
| | | identifier: MON-Ø1445-2). |
| B-65 | Bayer BioScience N.V., | Genetic elements which confer the phenotype |
| | Technologiepark 38 | insect resistant and glufosinate ammonium |
| | B-9052 Gent | herbicide tolerance: |
| | Belgium | cry1: Coding sequence of cry gene from Bacillus |
| | | thuringiensis that confers the insect resistance |
| | | trait. |
| | | bar: Coding sequence of the phosphinothricin |
| | | acetyltransferase gene (bar) from Streptomyces |
| | | hygroscopicus that confers the herbicide |
| | | resistance trait. |
| B-66 | Bayer BioScience N.V., | Genetic elements which confer the phenotype |
| | Technologiepark 38 | insect resistant and glufosinate ammonium |
| | B-9052 Gent | herbicide tolerance: |
| | Belgium | cry2: Coding sequence of cry gene from Bacillus |
| | | thuringiensis that confers the insect resistance |
| | | trait. |
| | | bar: Coding sequence of the phosphinothricin |
| | | acetyltransferase gene (bar) from Streptomyces |
| | | hygroscopicus that confers the herbicide |
| | | resistance trait. |
| B-67 | Bayer BioScience N.V., | Genetic elements which confer the phenotype |
| | Technologiepark 38 | insect resistant and glufosinate ammonium |
| | B-9052 Gent | herbicide tolerance: |
| | Belgium | cry2: Coding sequence of cry gene from Bacillus |
| | | thuringiensis that confers the insect resistance |
| | | trait. |
| | | bar: Coding sequence of the phosphinothricin |
| | | acetyltransferase gene (bar) from Streptomyces |
| | | hygroscopicus that confers the herbicide |
| | | resistance trait. |
| B-68 | Bayer BioScience N.V., | cry1: Coding sequence of cry gene from Bacillus |
| | Technologiepark 38 | thuringiensis that confers the insect resistance |
| | B-9052 Gent | trait. |
| | Belgium | bar: Coding sequence of the phosphinothricin |
| | | acetyltransferase gene (bar) from Streptomyces |
| | | hygroscopicus that confers the herbicide |
| | | resistance trait. |
| B-69 | Bayer BioScience N.V., | 2mepsps: Coding sequence of 2mepsps from |
| | Technologiepark 38 | maize that confers the glyphosate herbicide |
| | B-9052 Gent | resistance trait. |
| | Belgium | |
| B-70 | BASF Inc. | Tolerance to imidazolinone herbicides by |
| | | selection of a naturally occurring mutant. |
| B-71 | BASF Inc. | Selection for a mutagenized version of the |
| | | enzyme acetohydroxyacid synthase (AHAS), also |
| | | known as acetolactate synthase (ALS) or |
| | | acetolactate pyruvate-lyase. |
| B-72 | University of | A variant form of acetolactate synthase (ALS) |
| | Saskatchewan, Crop Dev. | was obtained from a chlorsulfuron tolerant line of |
| | Centre | A. thaliana and used to transform flax. |
| B-73 | Monsanto Company | Resistance to lepidopteran pests through the |
| | | introduction of the cry1Ac gene from Bacillus |
| | | thuringiensis subsp. Kurstaki. |
| B-74 | Monsanto Company | Introduction of a gene sequence encoding the |
| | | enzyme 1-amino-cyclopropane-1-carboxylic acid |
| | | deaminase (ACCd) that metabolizes the precursor |
| | | of the fruit ripening hormone ethylene. |
| B-75 | DNA Plant Technology | Delayed ripening tomatoes produced by inserting |
| | Corporation | an additional copy of a truncated gene encoding |
| | | 1-aminocyclopropane-1-carboxyllic acid (ACC) |
| | | synthase, which resulted in downregulation of the |
| | | endogenous ACC synthase and reduced ethylene |
| | | accumulation. |
| B-76 | Agritope Inc. | Introduction of a gene sequence encoding the |
| | | enzyme S-adenosylmethionine hydrolase that |
| | | metabolizes the precursor of the fruit ripening |
| | | hormone ethylene |
| B-77 | Zeneca Seeds | Delayed softening tomatoes produced by inserting |
| | | a truncated version of the polygalacturonase (PG) |
| | | encoding gene in the sense or anti-sense |
| | | orientation in order to reduce expression of the |
| | | endogenous PG gene, and thus reduce pectin |
| | | degradation. |
| B-78 | Calgene Inc. | Delayed softening tomatoes produced by inserting |
| | | an additional copy of the polygalacturonase (PG) |
| | | encoding gene in the anti-sense orientation in |
| | | order to reduce expression of the endogenous PG |
| | | gene and thus reduce pectin degradation. |
| B-79 | Monsanto Company and | Glyphosate herbicide tolerant alfalfa (lucerne) |
| | Forage Genetics | produced by inserting a gene encoding the |
| | International | enzyme 5-enolypyruvylshikimate-3-phosphate |
| | | synthase (EPSPS) from the CP4 strain of |
| | | Agrobacterium tumefaciens. |
| B-80 | Societe National | Tolerance to the herbicides bromoxynil and |
| | d'Exploitation des Tabacs | ioxynil by incorporation of the nitrilase gene from |
| | et Allumettes | Klebsiella pneumoniae. |
| B-81 | Vector Tobacco Inc. | Reduced nicotine content through introduction of |
| | | a second copy of the tobacco quinolinic acid |
| | | phosphoribosyltransferase (QTPase) in the |
| | | antisense orientation. The NPTII encoding gene |
| | | from E. coli was introduced as a selectable |
| | | marker to identify transformants. |
| B-82 | BASF Inc. | Tolerance to the imidazolinone herbicide, |
| | | imazethapyr, induced by chemical mutagenesis of |
| | | the acetolactate synthase (ALS) enzyme using |
| | | ethyl methanesulfonate (EMS). |
| B-83 | BASF Inc. | Tolerance to imidazolinone herbicides induced by |
| | | chemical mutagenesis of the acetolactate synthase |
| | | (ALS) enzyme using sodium azide. |
| B-84 | Aventis CropScience | Glufosinate ammonium herbicide tolerant rice |
| | | produced by inserting a modified |
| | | phosphinothricin acetyltransferase (PAT) |
| | | encoding gene from the soil bacterium |
| | | Streptomyces hygroscopicus). |
| B-85 | Bayer CropScience | Glufosinate ammonium herbicide tolerant rice |
| | (Aventis | produced by inserting a modified |
| | CropScience(AgrEvo)) | phosphinothricin acetyltransferase (PAT) |
| | | encoding gene from the soil bacterium |
| | | Streptomyces hygroscopicus). |
| B-86 | BASF Inc. | Tolerance to the imidazolinone herbicide, |
| | | imazethapyr, induced by chemical mutagenesis of |
| | | the acetolactate synthase (ALS) enzyme using |
| | | ethyl methanesulfonate (EMS). |
| B-87 | Monsanto Company | Colorado potato beetle resistant potatoes |
| | | produced by inserting the cry3A gene from |
| | | Bacillus thuringiensis (subsp. Tenebrionis). |
| B-88 | Monsanto Company | Colorado potato beetle resistant potatoes |
| | | produced by inserting the cry3A gene from |
| | | Bacillus thuringiensis (subsp. Tenebrionis). |
| B-89 | Monsanto Company | Colorado potato beetle and potato virus Y (PVY) |
| | | resistant potatoes produced by inserting the cry3A |
| | | gene from Bacillus thuringiensis (subsp. |
| | | Tenebrionis) and the coat protein encoding gene |
| | | from PVY. |
| B-90 | Monsanto Company | Colorado potato beetle and potato leafroll virus |
| | | (PLRV) resistant potatoes produced by inserting |
| | | the cry3A gene from Bacillus thuringiensis |
| | | (subsp. Tenebrionis) and the replicase encoding |
| | | gene from PLRV. |
| B-91 | BASF Plant Science | a) A gene containing the coding region of potato |
| | GmbH | gbss in antisense orientation relative to the |
| | | promoter, flanked by the gbss promoter from |
| | | Solanum tuberosum and the polyadenylation |
| | | sequence from Agrobacterium tumefaciens |
| | | nopaline synthase gene has been inserted into |
| | | potato variety Seresta (lines AM02-1003, AM01- |
| | | 1005, AM02-1012) and Kuras (line AM02-1017) |
| | | thus reducing the amount of amylose in the starch |
| | | fraction. An ahas gene (acetohydroxyacid |
| | | synthase) from Arabidopsis thaliana flanked by |
| | | the nos gene promoter and the octopine synthase |
| | | polyadenylation sequence from Agrobacterium |
| | | tumefaciens serves as selectable marker gene |
| | | conferring tolerance to Imazamox. |
| | | b) AM99-1089 serves as a reference line. The |
| | | inserted gene consists of the potato gbss (granule |
| | | bound starch synthase) promoter, the coding |
| | | region of potato gbss in antisense orientation and |
| | | the polyadenylation sequence from |
| | | Agrobacterium tumefaciens nopaline synthase |
| | | gene thus reducing the amount of amylose in the |
| | | starch fraction. In addition the neomycin |
| | | phosphotransferase gene (nptII) connected to the |
| | | Agrobacterium tumefaciens nopaline synthase |
| | | promoter and g7 polyadenylation sequence from |
| | | Agrobacterium tumefaciens has been inserted as |
| | | selectable marker gene conferring resistance to |
| | | kanamycin. |
| | | c) In potato line AM99-2003 a gene consisting of |
| | | gbss promoter from Solanum tuberosum, the |
| | | coding region fragments of be1 and be2 (starch- |
| | | branching enzyme) in tandem and antisense |
| | | orientation relative to the promoter and the nos |
| | | polyadenylation sequence from Agrobacterium |
| | | tumefaciens have been inserted into potato variety |
| | | Dinamo thus reducing the amount of amylopectin |
| | | in the starch fraction of the tuber. In addition the |
| | | neomycin phosphotransferase gene (nptII) |
| | | connected to the Agrobacterium tumefaciens |
| | | nopaline synthase promoter and g7 |
| | | polyadenylation sequence from Agrobacterium |
| | | tumefaciens has been inserted as selectable |
| | | marker gene conferring resistance to kanamycin. |
| B-92 | BASF Plant Science | In potato event EH92-527-1 a gene consisting of a |
| | GmbH | potato gbss (granule bound starch synthase) |
| | | promoter, a fragment of the coding region of |
| | | potato gbss in antisense orientation relative to the |
| | | promoter and the polyadenylation sequence from |
| | | Agrobacterium tumefaciens nopaline synthase |
| | | gene (gene construct pHoxwG) have been |
| | | inserted into potato variety Prevalent thus |
| | | reducing the amount of amylose in the starch |
| | | fraction. In addition the neomycin |
| | | phosphotransferase gene (nptII) connected to the |
| | | Agrobacterium tumefaciens nopaline synthase |
| | | promoter and polyadenylation signal has been |
| | | inserted as selectable marker gene conferring |
| | | resistance to kanamycin. |
| B-93 | BASF Inc. | Selection for a mutagenized version of the |
| | | enzyme acetohydroxyacid synthase (AHAS), also |
| | | known as acetolactate synthase (ALS) or |
| | | acetolactate pyruvate-lyase. |
| B-94 | BASF Inc. | Selection for a mutagenized version of the |
| | | enzyme acetohydroxyacid synthase (AHAS), also |
| | | known as acetolactate synthase (ALS) or |
| | | acetolactate pyruvate-lyase. |
| B-95 | BASF Inc. | Selection for a mutagenized version of the |
| | | enzyme acetohydroxyacid synthase (AHAS), also |
| | | known as acetolactate synthase (ALS) or |
| | | acetolactate pyruvate-lyase. |
| B-96 | Monsanto Company | Glyphosate tolerant wheat variety produced by |
| | | inserting a modified 5-enolpyruvylshikimate-3- |
| | | phosphate synthase (EPSPS) encoding gene from |
| | | the soil bacterium Agrobacterium tumefaciens, |
| | | strain CP4. |
| B-97 | Cyanamid Crop | Selection for a mutagenized version of the |
| | Protection | enzyme acetohydroxyacid synthase (AHAS), also |
| | | known as acetolactate synthase (ALS) or |
| | | acetolactate pyruvate-lyase. |
| B-98 | BASF Inc. | |
| B-99 | BASF Inc. | Tolerance to imidazolinone herbicides |
| B-100 | BASF Inc. | Selection for a mutagenized version of the |
| | | enzyme acetohydroxyacid synthase (AHAS), also |
| | | known as acetolactate synthase (ALS) or |
| | | acetolactate pyruvate-lyase. |
| B-101 | Syngenta Seeds, Inc., | Insect-resistant maize produced by inserting the |
| | Novartis, Mycogen | cry1Ab gene from Bacillus thuringiensis subsp. |
| | | kurstaki. The genetic modification affords |
| | | resistance to attack by the European corn borer |
| | | (ECB). |
| B-102 | Pioneer Hi-Bred | Selection of somaclonal variants by culture of |
| | International Inc. | embryos on imidazolinone containing media. |
| B-103 | Pioneer Hi-Bred | Male-sterile and glufosinate ammonium herbicide |
| | International Inc. | tolerant maize produced by inserting genes |
| | | encoding DNA adenine methylase and |
| | | phosphinothricin acetyltransferase (PAT) from |
| | | Escherichia coli and Streptomyces |
| | | viridochromogenes, respectively. |
| B-104 | Bayer CropScience | Stacked insect resistant and herbicide tolerant |
| | (Aventis | corn hybrid derived from conventional cross- |
| | CropScience(AgrEvo)) | breeding of the parental lines T25 (OECD |
| | | identifier: ACS-ZMØØ3-2) and MON810 (OECD |
| | | identifier: MON-ØØ81Ø-6). |
| B-105 | Dekalb Genetics | Glufosinate ammonium herbicide tolerant maize |
| | Corporation | produced by inserting the gene encoding |
| | | phosphinothricin acetyltransferase (PAT) from |
| | | Streptomyces hygroscopicus. |
| B-106 | Syngenta Seeds, Inc. | Insect-resistant and herbicide tolerant maize |
| | | produced by inserting the cry1Ab gene from |
| | | Bacillus thuringiensis subsp. kurstaki, and the |
| | | phosphinothricin N-acetyltransferase (PAT) |
| | | encoding gene from S. viridochromogenes. |
| B-107 | Aventis CropScience | Insect-resistant and glufosinate ammonium |
| | | herbicide tolerant maize developed by inserting |
| | | genes encoding Cry9C protein from Bacillus |
| | | thuringiensis subsp tolworthi and |
| | | phosphinothricin acetyltransferase (PAT) from |
| | | Streptomyces hygroscopicus. |
| B-108 | DOW AgroSciences LLC | Lepidopteran insect resistant and glufosinate |
| | | ammonium herbicide-tolerant maize variety |
| | | produced by inserting the cry1F gene from |
| | | Bacillus thuringiensis var aizawai and the |
| | | phosphinothricin acetyltransferase (PAT) from |
| | | Streptomyces hygroscopicus. |
| B-109 | DOW AgroSciences LLC | Corn rootworm-resistant maize produced by |
| | and Pioneer Hi-Bred | inserting the cry34Ab1 and cry35Ab1 genes from |
| | International Inc. | Bacillus thuringiensis strain PS149B1. The PAT |
| | | encoding gene from Streptomyces |
| | | viridochromogenes was introduced as a selectable |
| | | marker. |
| B-110 | DOW AgroSciences LLC | Stacked insect resistant and herbicide tolerant |
| | and Pioneer Hi-Bred | maize produced by conventional cross breeding of |
| | International Inc. | parental lines DAS-59122-7 (OECD unique |
| | | identifier: DAS-59122-7) with NK603 (OECD |
| | | unique identifier: MON-ØØ6Ø3-6). Corn |
| | | rootworm-resistance is derived from DAS-59122- |
| | | 7 which contains the cry34Ab1 and cry35Ab1 |
| | | genes from Bacillus thuringiensis strain PS149B1. |
| | | Tolerance to glyphosate herbcicide is derived |
| | | from NK603. |
| B-111 | DOW AgroSciences LLC | Stacked insect resistant and herbicide tolerant |
| | and Pioneer Hi-Bred | maize produced by conventional cross breeding of |
| | International Inc. | parental lines DAS-59122-7 (OECD unique |
| | | identifier: DAS-59122-7) and TC1507 (OECD |
| | | unique identifier: DAS-Ø15Ø7-1) with NK603 |
| | | (OECD unique identifier: MON-ØØ6Ø3-6). Corn |
| | | rootworm-resistance is derived from DAS-59122- |
| | | 7 which contains the cry34Ab1 and cry35Ab1 |
| | | genes from Bacillus thuringiensis strain PS149B1. |
| | | Lepidopteran resistance and toleraance to |
| | | glufosinate ammonium herbicide is derived from |
| | | TC1507. Tolerance to glyphosate herbcicide is |
| | | derived from NK603. |
| B-112 | DOW AgroSciences LLC | Stacked insect resistant and herbicide tolerant |
| | | corn hybrid derived from conventional cross- |
| | | breeding of the parental lines 1507 (OECD |
| | | identifier: DAS-Ø15Ø7-1) and NK603 (OECD |
| | | identifier: MON-ØØ6Ø3-6). |
| B-113 | Dekalb Genetics | Insect-resistant and glufosinate ammonium |
| | Corporation | herbicide tolerant maize developed by inserting |
| | | genes encoding Cry1AC protein from Bacillus |
| | | thuringiensis subsp kurstaki and phosphinothricin |
| | | acetyltransferase (PAT) from Streptomyces |
| | | hygroscopicus |
| B-114 | BASF Inc. | Somaclonal variants with a modified acetyl-CoA- |
| | | carboxylase (ACCase) were selected by culture of |
| | | embryos on sethoxydim enriched medium. |
| B-115 | Syngenta Seeds, Inc. | Tolerance to the imidazolinone herbicide, |
| | (formerly Zeneca Seeds) | imazethapyr, induced by chemical mutagenesis of |
| | | the acetolactate synthase (ALS) enzyme using |
| | | ethyl methanesulfonate (EMS). |
| B-116 | Monsanto Company | Introduction, by particle bombardment, of a |
| | | modified 5-enolpyruvyl shikimate-3-phosphate |
| | | synthase (EPSPS), an enzyme involved in the |
| | | shikimate biochemical pathway for the production |
| | | of the aromatic amino acids. |
| B-117 | Pioneer Hi-Bred | Tolerance to the imidazolinone herbicide, |
| | International Inc. | imazethapyr, was obtained by in vitro selection of |
| | | somaclonal variants. |
| B-118 | Monsanto Company | Altered amino acid composition, specifically |
| | | elevated levels of lysine, through the introduction |
| | | of the cordapA gene, derived from |
| | | Corynebacterium glutamicum, encoding the |
| | | enzyme dihydrodipicolinate synthase (cDHDPS). |
| B-119 | Syngenta Seeds, Inc. | Corn rootworm resistant maize produced by |
| | | transformation with a modified cry3A gene. The |
| | | phosphomannose isomerase gene from E. coli was |
| | | used as a selectable marker. |
| B-120 | Monsanto Company | Insect-resistant maize produced by inserting the |
| | | cry1Ab gene from Bacillus thuringiensis subsp. |
| | | kurstaki. The genetic modification affords |
| | | resistance to attack by the European corn borer |
| | | (ECB). |
| B-121 | Monsanto Company | Insect-resistant and glyphosate herbicide tolerant |
| | | maize produced by inserting the genes encoding |
| | | the Cry1Ab protein from Bacillus thuringiensis |
| | | and the 5-enolpyruvylshikimate-3-phosphate |
| | | synthase (EPSPS) from A. tumefaciens strain |
| | | CP4. |
| B-122 | Pioneer Hi-Bred | Resistance to European corn borer (Ostrinia |
| | International Inc. | nubilalis) by introduction of a synthetic cry1Ab |
| | | gene. Glyphosate resistance via introduction of |
| | | the bacterial version of a plant enzyme, 5- |
| | | enolpyruvyl shikimate-3-phosphate synthase |
| | | (EPSPS). |
| B-123 | Monsanto Company | Insect-resistant maize produced by inserting a |
| | | truncated form of the cry1Ab gene from Bacillus |
| | | thuringiensis subsp. kurstaki HD-1. The genetic |
| | | modification affords resistance to attack by the |
| | | European corn borer (ECB). |
| B-124 | Monsanto Company | Stacked insect resistant and glyphosate tolerant |
| | | maize derived from conventional cross-breeding |
| | | of the parental lines MON810 (OECD identifier: |
| | | MON-ØØ81Ø-6) and MON88017 (OECD |
| | | identifier: MON-88Ø17-3). European corn borer |
| | | (ECB) resistance is derived from a truncated form |
| | | of the cry1Ab gene from Bacillus thuringiensis |
| | | subsp. kurstaki HD-1 present in MON810. Corn |
| | | rootworm resistance is derived from the cry3Bb1 |
| | | gene from Bacillus thuringiensis subspecies |
| | | kumamotoensis strain EG4691 present in |
| | | MON88017. Glyphosate tolerance is derived from |
| | | a 5-enolpyruvylshikimate-3-phosphate synthase |
| | | (EPSPS) encoding gene from Agrobacterium |
| | | tumefaciens strain CP4 present in MON88017. |
| B-125 | Monsanto Company | Introduction, by particle bombardment, of |
| | | glyphosate oxidase (GOX) and a modified 5- |
| | | enolpyruvyl shikimate-3-phosphate synthase |
| | | (EPSPS), an enzyme involved in the shikimate |
| | | biochemical pathway for the production of the |
| | | aromatic amino acids. |
| B-126 | Monsanto Company | Corn root worm resistant maize produced by |
| | | inserting the cry3Bb1 gene from Bacillus |
| | | thuringiensis subsp. kumamotoensis. |
| B-127 | Monsanto Company | Corn rootworm-resistant maize produced by |
| | | inserting the cry3Bb1 gene from Bacillus |
| | | thuringiensis subspecies kumamotoensis strain |
| | | EG4691. Glyphosate tolerance derived by |
| | | inserting a 5-enolpyruvylshikimate-3-phosphate |
| | | synthase (EPSPS) encoding gene from |
| | | Agrobacterium tumefaciens strain CP4. |
| B-128 | Monsanto Company | Stacked insect resistant and herbicide tolerant |
| | | corn hybrid derived from conventional cross- |
| | | breeding of the parental lines NK603 (OECD |
| | | identifier: MON-ØØ6Ø3-6) and MON810 |
| | | (OECD identifier: MON-ØØ81Ø-6). |
| B-129 | Monsanto Company | Stacked insect resistant and enhanced lysine |
| | | content maize derived from conventional cross- |
| | | breeding of the parental lines MON810 (OECD |
| | | identifier: MON-ØØ81Ø-6) and LY038 (OECD |
| | | identifier: REN-ØØØ38-3). |
| B-130 | Monsanto Company | Stacked insect resistant and herbicide tolerant |
| | | corn hybrid derived from conventional cross- |
| | | breeding of the parental lines MON863 (OECD |
| | | identifier: MON-ØØ863-5) and NK603 (OECD |
| | | identifier: MON-ØØ6Ø3-6). |
| B-131 | Monsanto Company | Stacked insect resistant corn hybrid derived from |
| | | conventional cross-breeding of the parental lines |
| | | MON863 (OECD identifier: MON-ØØ863-5) and |
| | | MON810 (OECD identifier: MON-ØØ81Ø-6) |
| B-132 | Monsanto Company | Stacked insect resistant and herbicide tolerant |
| | | corn hybrid derived from conventional cross- |
| | | breeding of the stacked hybrid MON-ØØ863-5 × |
| | | MON-ØØ81Ø-6 and NK603 (OECD |
| | | identifier: MON-ØØ6Ø3-6). |
| B-133 | Monsanto Company | Stacked insect resistant and herbicide tolerant |
| | | corn hybrid derived from conventional cross- |
| | | breeding of the parental lines GA21 (OECD |
| | | identifider: MON-ØØØ21-9) and MON810 |
| | | (OECD identifier: MON-ØØ81Ø-6). |
| B-134 | Bayer CropScience | Male sterility caused by expression of the barnase |
| | (Aventis | ribonuclease gene from Bacillus |
| | CropScience(AgrEvo)) | amyloliquefaciens; PPT resistance was via PPT- |
| | | acetyltransferase (PAT). |
| B-135 | Bayer CropScience | Male sterility caused by expression of the barnase |
| | (Aventis | ribonuclease gene from Bacillus |
| | CropScience(AgrEvo)) | amyloliquefaciens; PPT resistance was via PPT- |
| | | acetyltransferase (PAT). |
| B-136 | Monsanto Company | Introduction, by particle bombardment, of a |
| | | modified 5-enolpyruvyl shikimate-3-phosphate |
| | | synthase (EPSPS), an enzyme involved in the |
| | | shikimate biochemical pathway for the production |
| | | of the aromatic amino acids. |
| B-137 | Syngenta Seeds, Inc. | Stacked insect resistant and herbicide tolerant |
| | | maize produced by conventional cross breeding of |
| | | parental lines BT11 (OECD unique identifier: |
| | | SYN-BTØ11-1) and GA21 (OECD unique |
| | | identifier: MON-ØØØ21-9). |
| B-138 | Bayer CropScience | Glufosinate herbicide tolerant maize produced by |
| | (Aventis | inserting the phosphinothricin N-acetyltransferase |
| | CropScience(AgrEvo)) | (PAT) encoding gene from the aerobic |
| | | actinomycete Streptomyces viridochromogenes. |
| B-139 | Mycogen (c/o Dow | Insect-resistant and glufosinate ammonium |
| | AgroSciences); Pioneer | herbicide tolerant maize produced by inserting the |
| | (c/o Dupont) | cry1F gene from Bacillus thuringiensis var. |
| | | aizawai and the phosphinothricin N- |
| | | acetyltransferase encoding gene from |
| | | Streptomyces viridochromogenes. |
| B-140 | DOW AgroSciences LLC | Stacked insect resistant and herbicide tolerant |
| | and Pioneer Hi-Bred | maize produced by conventional cross breeding of |
| | International Inc. | parental lines TC1507 (OECD unique identifier: |
| | | DAS-Ø15Ø7-1) with DAS-59122-7 (OECD |
| | | unique identifier: DAS-59122-7). Resistance to |
| | | lepidopteran insects is derived from TC1507 due |
| | | the presence of the cry1F gene from Bacillus |
| | | thuringiensis var. aizawai. Corn rootworm- |
| | | resistance is derived from DAS-59122-7 which |
| | | contains the cry34Ab1 and cry35Ab1 genes from |
| | | Bacillus thuringiensis strain PS149B1. Tolerance |
| | | to glufosinate ammonium herbcicide is derived |
| | | from TC1507 from the phosphinothricin N- |
| | | acetyltransferase encoding gene from |
| | | Streptomyces viridochromogenes. |
| B-141 | Syngenta Agrisure GT | Glyphosate Herbicide Tolerance |
| B-142 | Syngenta Agrisure | Cry1Ab Corn borer protection |
| | GT/CB YieldGard | Glyphosate Herbicide Tolerance |
| | Liberty Link | |
| B-143 | MonsantoYieldGard | Cry1Ab corn borer resistance |
| | Roundup Ready | Glyphosate Herbicide Tolerance |
| B-144 | Monsanto Agrar | A full description of the genetic elements in MON |
| | Deutschland GmbH | 89034, including the approximate size, source and |
| | | function is provided in Table 1. |
| | | Table 1. Summary of the genetic elements |
| | | inserted in MON 89034 |
| | | B1-Left Border*: 239 bp DNA region from the |
| | | B?Left Border region remaining after integration |
| | | Pp2-e35S: Modified promoter and leader for the |
| | | cauliflower mosaic virus (CaMV) 35S RNA |
| | | containing the duplicated enhancer region |
| | | L3-Cab: 5′ untranslated leader of the wheat |
| | | chlorophyll a/b?binding protein |
| | | I4-Ract1: Intron from the rice actin gene |
| | | CS5-cry1A.105: Coding sequence for the |
| | | Bacillus thuringiensis Cry1A.105 protein |
| | | T6-Hsp17: 3′ transcript termination sequence for |
| | | wheat heat shock protein 17.3, which ends |
| | | transcription and directs polyadenylation |
| | | P-FMV: Figwort Mosaic Virus 35S promoter |
| | | I-Hsp70: First intron from the maize heat shock |
| | | protein 70 gene |
| | | TS7-SSU-CTP: DNA region containing the |
| | | targeting sequence for the transit peptide region |
| | | of maize ribulose 1,5-bisphosphate carboxylase |
| | | small subunit and the first intron |
| | | CS-cry2Ab2: Coding sequence for a Cry2Ab2 |
| | | protein from Bacillus thuringiensis. This coding |
| | | sequence uses a modified codon usage. |
| | | T-nos: 3′ transcript termination sequence of the |
| | | nopaline synthase (nos) coding sequence from |
| | | Agrobacterium tumefaciens which terminates |
| | | transcription and directs polyadenylation |
| | | B-Left Border: 230 bp DNA region from the B- |
| | | Left Border region remaining after integration |
| | | *Analyses of the MON 89034 insert sequence |
| | | revealed that the e35S promoter that regulates |
| | | expression of the cry1A.105 coding sequence was |
| | | modified: the Right Border sequence present in |
| | | PV-ZMIR245 was replaced by the Left Border |
| | | sequence. It is likely that this modification is the |
| | | result of a crossover recombination event that |
| | | occurred prior to the DNA being inserted into the |
| | | genome. |
| B-145 | Monsanto Agrar | |
| | Deutschland GmbH | |
| B-146 | Monsanto Agrar | |
| | Deutschland GmbH | |
| B-147 | Pioneer Hi-Bred Seeds | 98140 maize has been genetically modified by |
| | Agro SRL | insertion of the glyphosate-N-acetyltransferase |
| | | (gat4621) gene and a modified maize acetolactate |
| | | synthase (zm-hra) gene, along with the necessary |
| | | regulatory elements for gene expression in the |
| | | maize plant. |
| | | The gat4621 gene encodes the GAT4621 protein, |
| | | which was derived from the soil bacterium |
| | | Bacillus licheniformis, and confers tolerance to |
| | | herbicides containing glyphosate. The zm-hra |
| | | gene encodes the ZM-HRA protein and confers |
| | | tolerance to a range of ALS-inhibiting herbicides |
| | | such as sulfonylureas. |
| B-148 | Syngenta Seeds SA | Regulatory sequences: |
| | | Promoter sequences derived from maize. The |
| | | function of these sequences is to control |
| | | expression of the insect resistance gene. |
| | | Insect resistance gene: |
| | | cry1Ab gene derived form Bacillus thuringiensis. |
| | | The function of the product of this gene is to |
| | | confer resistance to certain lepidopteran pests. |
| | | NOS terminator: |
| | | Terminator sequence of the nopaline synthase |
| | | gene, isolated from Agrobacterium tumefaciens. |
| | | The function of this sequence is to signal the |
| | | termination of the insect resistance gene |
| | | expression. |
| | | ZmUbilntron: |
| | | Promoter from a maize ubiquitin gene together |
| | | with the first intron of the gene. The function of |
| | | these sequences is to control and enhance |
| | | expression of the Phosphomannose Isomerase |
| | | (pmi) gene. |
| | | pmi: |
| | | Coding sequence of the Phosphomannose |
| | | Isomerase (pmi) gene isolated from Escherichia |
| | | coli. The function of this gene product is as a |
| | | selectable marker for the transformation, as it |
| | | allows positive selection of transformed cells |
| | | growing on mannose. |
| | | NOS terminator: |
| | | Termination sequence of the nopaline synthase |
| | | gene, isolated from Agrobacterium tumefaciens. |
| | | The function of this sequence is to signal the |
| | | termination of the marker gene (pmi) expression. |
| B-149 | Delta and Pine Land | Bollgard ®, RoundupReady ® |
| | company | |
| B-150 | | |
| B-151 | Monsanto Company | |
| B-152 | Monsanto Company | |
| B-153 | | |
| B-154 | Illinois Foundation Seeds | |
| B-155 | | |

TABLE 6

| No. | Commercial name | Plant | Company |
|---|---|---|---|
| 4-1 | Roundup Ready ® | Beta vulgaris (Sugar Beet) | Monsanto Company |
| 4-2 | InVigor ® | Brassica napus (Argentine Canola) | BayerCropScience |
| 4-3 | Liberty Link ® | Brassica napus (Argentine Canola) | BayerCropScience |
| 4-4 | Roundup Ready ® | Brassica napus (Canola) | Monsanto Company |
| 4-5 | Clearfield ® | Canola | BASF Corporation |
| 4-6 | Optimum ™ GAT ™ | Glycine max | Pioneer Hi-Bred International, Inc |
| | | L. (Soybean) | |
| 4-7 | Roundup Ready ® | Glycine max | Monsanto Company |
| | | L. (Soybean) | |
| 4-8 | Roundup | Glycine max | Monsanto Company |
| | RReady2Yield ™ | L. (Soybean) | |
| 4-9 | STS ® | Glycine max | DuPont |
| | | L. (Soybean) | |
| 4-10 | YIELD GARD ® | Glycine max | Monsanto Company |
| | | L. (Soybean) | |
| 4-11 | AFD ® | Gossypium hirsutum L. (Cotton) | BayerCropScience |
| 4-12 | Bollgard II ® | Gossypium hirsutum | Monsanto Company |
| | | L. (Cotton) | |
| 4-13 | Bollgard ® | Gossypium hirsutum | Monsanto Company |
| | | L. (Cotton) | |
| 4-14 | FiberMax ® | Gossypium hirsutum | BayerCropScience |
| | | L. (Cotton) | |
| 4-15 | Liberty Link ® | Gossypium hirsutum | BayerCropScience |
| | | L. (Cotton) | |
| 4-16 | Nucotn 33B | Gossypium hirsutum | Delta Pine and Land |
| | | L. (Cotton) | |
| 4-17 | Nucotn 35B | Gossypium hirsutum | Delta Pine and Land |
| | | L. (Cotton) | |
| 4-18 | Nucotn ® | Gossypium hirsutum | Delta Pine and Land |
| | | L. (Cotton) | |
| 4-19 | PhytoGen ™ | Gossypium hirsutum | PhytoGen |
| | | L. (Cotton) | Seed Company, Dow AgroSciences LLC |
| 4-20 | Roundup Ready | Gossypium hirsutum | Monsanto Company |
| | Flex ® | L. (Cotton) | |
| 4-21 | Roundup Ready ® | Gossypium hirsutum | Monsanto Company |
| | | L. (Cotton) | |
| 4-22 | Widestrike ™ | Gossypium hirsutum | Dow AgroSciences |
| | | L. (Cotton) | LLC |
| 4-23 | YIELD GARD ® | Gossypium hirsutum | Monsanto Company |
| | | L. (Cotton) | |
| 4-24 | Roundup Ready ® | Medicago | Monsanto Company |
| | | sativa (Alfalfa) | |
| 4-25 | Clearfield ® | Oryza sativa (Rice) | BASF Corporation |
| 4-26 | NewLeaf ® | Solanum tuberosum | Monsanto Company |
| | | L. (Potato) | |
| 4-27 | NewLeaf ® plus | Solanum tuberosum | Monsanto Company |
| | | L. (Potato) | |
| 4-28 | Protecta ® | Solanum tuberosum | ? |
| | | L. (Potato) | |
| 4-29 | Clearfield ® | Sunflower | BASF Corporation |
| 4-30 | Roundup Ready ® | Triticum | Monsanto Company |
| | | aestivum (Wheat) | |
| 4-31 | Clearfield ® | Wheat | BASF Corporation |
| 4-32 | Agrisure ® (Family) | Zea mays L. (Maize) | Syngenta Seeds, Inc. |
| 4-33 | BiteGard ® | Zea mays L. (Maize) | Novartis Seeds |
| 4-34 | Bt-Xtra ® | Zea mays L. (Maize) | DEKALB Genetics |
| | | | Corporation |
| 4-35 | Clearfield ® | Zea mays L. (Maize) | BASF Corporation |
| 4-36 | Herculex ® (Family) | Zea mays L. (Maize) | Dow AgroSciences |
| | | | LLC |
| 4-37 | IMI ® | Zea mays L. (Maize) | DuPont |
| 4-38 | KnockOut ® | Zea mays L. (Maize) | Syngenta Seeds, Inc. |
| 4-39 | Mavera ® | Zea mays L. (Maize) | Renessen LLC |
| 4-40 | NatureGard ® | Zea mays L. (Maize) | Mycogen |
| 4-41 | Roundup Ready ® | Zea mays L. (Maize) | Monsanto Company |
| 4-42 | Roundup Ready ® 2 | Zea mays L. (Maize) | Monsanto Company |
| 4-43 | SmartStax | Zea mays L. (Maize) | Monsanto Company |
| 4-44 | StarLink ® | Zea mays L. (Maize) | Aventis CropScience |
| | | | ->Bayer CropScience |
| 4-45 | STS ® | Zea mays L. (Maize) | DuPont |
| 4-46 | YIELD GARD ® | Zea mays L. (Maize) | Monsanto Company |
| 4-47 | YieldGard ® Plus | Zea mays L. (Maize) | Monsanto Company |
| 4-48 | YieldGard ® | Zea mays L. (Maize) | Monsanto Company |
| | Rootworm | | |
| 4-49 | YieldGard ® VT | Zea mays L. (Maize) | Monsanto Company |
| 4-50 | YieldMaker ™ | Zea mays L. (Maize) | DEKALB Genetics |
| | | | Corporation |

| No. | Transgenically modified properties | Additional information |
|---|---|---|
| 4-1 | tolerance to glyphosate | |
| 4-2 | Canola has been genetically modified | |
| | to: | |
| | Ø express a gene conferring tolerance | |
| | to the herbicide glufosinate | |
| | ammonium; | |
| | Ø introduce a novel hybrid breeding | |
| | system for canola, based on | |
| | genetically modified male | |
| | sterile (MS) and fertility restorer (RF) | |
| | lines; | |
| | Ø express an antibiotic resistance | |
| | gene. | |
| 4-3 | tolerance to phosphinotricin | |
| 4-4 | tolerance to glyphosate | |
| 4-5 | non-GMO, tolerance to imazamox | |
| 4-6 | tolerance to glyphosate and ALS | |
| | herbicides | |
| 4-7 | tolerance to glyphosate | |
| 4-8 | tolerance to glyphosate | |
| 4-9 | tolerance to sulphonylureas | |
| 4-10 | | |
| 4-11 | lines include eg AFD5062LL, | |
| | AFD5064F, AFD 5065B2F, AFD seed | |
| | is available in several varieties with | |
| | technology incorporated, such as | |
| | Bollgard ®, Bollgard II, Roundup | |
| | Ready, Roundup Ready Flex and | |
| | LibertyLink ® technologies. | |
| 4-12 | MON 15985 event: Cry2(A)b1; | |
| | Cry1A(c) | |
| 4-13 | Cry 1Ac | |
| 4-14 | | |
| 4-15 | tolerance to phosphinotricin | |
| 4-16 | Bt-toxin in Delta Pine lines: CrylAc | |
| 4-17 | Bt-toxin in Delta Pine lines: CrylAc | |
| 4-18 | Bt-toxin in Delta Pine lines | |
| 4-19 | covers varieties containing for | |
| | example Roundup Ready flex, | |
| | Widestrike, | |
| 4-20 | tolerance to glyphosate | |
| 4-21 | tolerance to glyphosate | |
| 4-22 | Cry1F and Cry1Ac | Monsanto/Dow |
| 4-23 | | http://www.garstseed.com/GarstClient/Technology/agrisure.aspx |
| 4-24 | tolerance to glyphosate | |
| 4-25 | non-GMO, tolerance to imazamox | |
| 4-26 | resistant to infection by Potato | |
| | Leafroll Virus (PLRV) and to feeding | |
| | by the Colorado potato beetle, | |
| | Leptinotarsa decemlineata (CPB) | |
| 4-27 | resistant to infection by Potato | http://www.dowagro.com/phytogen/index.htm |
| | Leafroll Virus (PLRV) and to feeding | |
| | by the Colorado potato beetle, | |
| | Leptinotarsa decemlineata (CPB) | |
| 4-28 | | |
| 4-29 | non-GMO, tolerance to imazamox | |
| 4-30 | tolerance to glyphosate, NK603 | |
| 4-31 | non-GMO, tolerance to imazamox | |
| 4-32 | includes Agrisure CB/LL (BT 11 | |
| | event plus tolerance towards | |
| | phosphinotricin by GA21 event); | |
| | Agrisure CB/LL/RW (Bt 11 event, | |
| | modified synthetic Cry3A gene, | |
| | tolerance towards phosphinotricin by | |
| | GA21 event); Agrisure GT (tolerance | |
| | to glyphosate); Agrisure | |
| | GT/CB/LL(tolerance to glyphosate | |
| | and towards phosphinotricinby GA21 | |
| | event, Bt 11 event); Agrisure 3000GT | |
| | (CB/LL/RW/GT: tolerance to | |
| | glyphosate and towards | |
| | phosphinotricinby GA21 event, Bt 11 | |
| | event, modified synthetic Cry3A gene); | |
| | Agrisure GT/RW (tolerance to | |
| | glyphosate, modified synthetic Cry3A | |
| | gene); Agrisure RW (modified | |
| | synthetic Cry3A gene); Future Traits | |
| 4-33 | cry1A(b) gene. | |
| 4-34 | cry1Ac gene. | |
| 4-35 | non-GMO, tolerance to imazamox | |
| 4-36 | | |
| 4-37 | tolerance to imidazolinones | |
| 4-38 | SYN-EV176-9: cry1A(b) gene. | |
| 4-39 | high Lysine | http://www.dowagro.com/widestrike/ |
| 4-40 | cry1A(b) gene. | |
| 4-41 | tolerance to glyphosate | http://www.starlinkcorn.com/starlinkcorn.htm |
| 4-42 | tolerance to glyphosate | |
| 4-43 | eight gene stack | |
| 4-44 | Cry9c gene. | |
| 4-45 | tolerance to sulphonylureas | |
| 4-46 | Mon810, Cry1Ab1; resistant to corn | http://www.dowagro.com/herculex/about/herculexfamily/ |
| | borer | |
| 4-47 | Mon810 × Mon863, double-stack, | |
| | resistant to corn borer and rootworm | |
| 4-48 | Mon863, Cry3Bb1, resistant to | |
| | rootworm | |
| 4-49 | stacked trait | |
| 4-50 | include Roundup Ready 2 technology, | |
| | YieldGard VT, YieldGard Corn Borer, | |
| | YieldGard Rootworm and YieldGard | |
| | Plus | |

### EXAMPLES

The invention is illustrated in more detail by the examples below, without being limited thereby.

### Example 1

Individually potted transgenic cotton plants with Lepidoptera resistance and herbicide resistance (line DP444 BG/RR) are treated against larvae of the cotton bollworm (Heliothis armigera) in two replications. Application is by spray application with the active compound in question at the stated application rate.

After the desired period of time, the kill in % is determined. 100% means that all caterpillars have been killed; 0% means that none of the caterpillars have been killed.

A considerable improvement in the control of pests compared to the control plants not treated according to the invention is noticeable.

### Example 2

Pots containing in each case 5 transgenic maize plants with Lepidoptera resistance and herbicide resistance (line SGI1890 Hx×AGI1847) are treated against the army worm (Spodoptera frugiperda) in 2 replications. Application is by spray application with the active compound in question at the stated application rate.

After the desired period of time, the kill in % is determined. 100% means that all caterpillars have been killed; 0% means that none of the caterpillars have been killed.

A considerable improvement in the control of pests compared to the control plants not treated according to the invention is noticeable.

### Example 3

Pots containing in each case 5 transgenic maize plants with herbicide resistance (line FR1064LL X FR2108) are treated against the army worm (Spodoptera frugiperda) in 2 replications. Application is by spray application with the active compound in question at the stated application rate.

After the desired period of time, the kill in % is determined. 100% means that all caterpillars have been killed; 0% means that none of the caterpillars have been killed.

A considerable improvement in the control of pests compared to the control plants not treated according to the invention is noticeable.

### Examples 4 to 6

The invention is furthermore also illustrated in more detail by the examples below, without being limited thereby. The spirotetramate mentioned in the tables is the compound I-4.

The activity according to the invention, i.e. the synergistic activity between the transgenic property of the plant and the active compound treatment can be demonstrated using the method of S.R. Colby, Weeds 15 (1967), 20-22. This is based on the following calculation base and assumption (“Colby formula”):

If
X is the kill rate, expressed in % of the untreated and not transgenically modified control, when employing the active compound A at an application rate of m g/ha or at a concentration of m ppm,
Y is the kill rate, expressed in % of the untreated and not transgenically modified control, when using a transgenic plant and
E is the kill rate, expressed in % of the untreated and not transgenically modified control, when employing active compound A and using a transgenically modified plant, the application rate being m and n g/ha or the concentration being m and n ppm,
   then

$$E = X + Y - \frac{X \cdot Y}{100} .$$

If the actual kill rate (i.e. the activity observed) is higher than the calculated one, the combination of active compound treatment and transgenically modified plant is superadditive in its kill, i.e. a synergistic effect between the active compound treatment and the use of a transgenic plant is present. In this case, the actually observed kill rate must thus be higher than the value calculated using the formula above for the kill rate (E).

In Examples 4 to 6 below, the observed kill rate is higher than the calculated kill rate. Thus, the synergistic activity according to the invention is present. According to the method according to the invention, four days after the treatment a kill of harmful organisms of at least 20%, preferably at least 30%, in particular at least 50%, compared to the control, can be observed. It is also possible to achieve kill results of at least 80 or 90% four days after treatment. Even one day after treatment, the kill of harmful organisms may be at least 20 or 30%.

### Example 4

Individually potted transgenic cotton plants having a Lepidoptera resistance and a herbicide resistance (line DP444 BG/RR) which are populated by a mixed population of the cotton aphid (Aphis gossypii) are treated with the active compound in question by spray application.

After a desired period of time, the kill in % is determined by counting the animals. 100% means that all aphids have been killed; 0% means that none of the aphids have been killed.

Compared to the control plants not treated according to the invention, a marked improvement in the control of the pests can be noticed.

| Active | | | |
|---|---|---|---|
| compound and line | Concentration | Kill | |
| of the transgenic plant | in ppm | in % after 4^{d} | |
| Spirotetramate | 100 | 35 | |
| DP 444 BG/RR | | 0 | |
| Cry1Ac&cp4 epsps | | | |
| | | observed* | calculated** |
| Spirotetramate + DP 444 | 100 | 55 | 35 |
| BG/RR | | | |

*observed according to the invention = activity found
**calculated = activity calculated using the “Colby formula”

### Example 5

In two replications, pots with in each case 5 transgenic maize plants having a Coleoptera, Lepidoptera and/or a herbicide resistance (lines LH332RR×LH324BT, HC33CRW×LH287BTCRW, HCL201CRW2RR×LH324 and FR1064LL×FR2108, respectively) are treated against the armyworm (Spodoptera frugiperda). Application is by spray application with the active compound in question at the desired application rate.

After a desired period of time, the kill in % is determined by counting the animals. 100% means that all caterpillars have been killed; 0% means that none of the caterpillars have been killed.

Compared to the control plants not treated according to the invention, a marked improvement in the control of the pests can be noticed.

| Active | | | |
|---|---|---|---|
| compound and line of | Concentration | Kill | |
| the transgenic plant | in ppm | in % after 1^{d} | |
| Spirotetramate | 100 | 0 | |
| VSN-BTCRW | | 0 | |
| Cry1Ab&Cry3Bb1 | | | |
| HCL201CRW2RR × LH | | 0 | |
| 324 | | | |
| Cry3Bb1&CP4epsps | | | |
| | | found* | calculated* |
| Spirotetramate + VSN- | 100 | 20 | 0 |
| BTCRW according to | | | |
| the invention | | | |
| | | observed* | calculated** |
| Spirotetramate + | 100 | 30 | 0 |
| HCL201CRW2RR × LH | | | |
| 324 | | | |

*observed according to the invention = activity found
**calculated = activity calculated using the “Colby formula”

| Active | | | |
|---|---|---|---|
| compound and line of | Concentration | Kill | |
| the transgenic plant | in ppm | in % after 4^{d} | |
| Spirotetramate | 100 | 60 | |
| VSN-RR Bt | | 0 | |
| Cry1Ab&Cp4 epsps | | | |
| FR1064LL × FR2108 | | 10 | |
| Glufosinate ammonium | | | |
| resistance | | | |
| | | observed* | calculated** |
| Spirotetramate + VSN- | 100 | 90 | 60 |
| RR Bt according to | | | |
| the invention | | | |
| | | observed* | calculated** |
| Spirotetramate + FR | 100 | 80 | 64 |
| 1064LL × FR2108 | | | |

*observed according to the invention = activity found
**calculated = activity calculated using the “Colby formula”

### Example 6

In two replications, pots with in each case 5 transgenic maize plants having a Coleoptera, Lepidoptera and/or a herbicide resistance (lines HC33CRW×LH287BTCRW and TR 47×TR 7322 BT, respectively) are treated against larvae of the armyworm (Spodoptera exigua). Application is by spray application with the active compound in question at the desired application rate.

After a desired period of time, the kill in % is determined by counting the animals. 100% means that all caterpillars have been killed; 0% means that none of the caterpillars have been killed.

Compared to the control plants not treated according to the invention, a marked improvement in the control of the pests can be noticed.

| Active | | | |
|---|---|---|---|
| compound and line of | Concentration | Kill | |
| the transgenic plant | in ppm | in % after 4^{d} | |
| Spirotetramate | 100 | 10 | |
| VSN-BTCRW | | 60 | |
| Cry1Ab&Cry3Bb1 | | | |
| VSN-BT | | 10 | |
| Bt MON 810 | | | |
| | | observed* | calculated** |
| Spirotetramate + VSN- | 100 | 90 | 64 |
| BTCRW according to the | | | |
| invention | | | |
| | | observed* | calculated** |
| Spirotetramate + VSN- | 100 | 30 | 19 |
| BT | | | |

*observed according to the invention = activity found
**calculated = activity calculated using the “Colby formula”

1. A method for improving the utilization of the production potential of a transgenic plant, comprising treating the plant with an effective amount of at least one 3-arylpyrrolidine-2,4-dione derivative.

2. A method according to claim 1, wherein said 3-arylpyrrolidine-2,4-dione derivative is a compound of formula I.

3. A method according to claim 1, wherein said 3-arylpyrrolidine-2,4-dione derivative is a compound of formulae I-1 to I-13.

4. A method according to claim 3, wherein said 3-arylpyrrolidine-2,4-dione derivative is a substantially pure cis isomer of the compounds I-3 and/or I-4.

5. A method according to claim 1, wherein the plant has at least one genetically modified structure or a tolerance according to Table 1.

6. A method according to claim 1, wherein the plant has at least one modified principle of action according to Table 3.

7. A method according to claim 1, wherein the plant is a transgenic plant according to one of Tables 4 to 6.

8. A method according to claim 1, wherein the plant contains at least one genetic modification according to Table 2.

9. A method according to claim 1, wherein the transgenic plant contains at least one gene or a gene fragment coding for a Bt toxin.

10. A method according to claim 1, wherein the transgenic plant is a vegetable plant, maize plant, soyabean plant, cotton plant, tobacco plant, rice plant, sugar beet plant or potato plant.

11. A method according to claim 1, wherein the 3-arylpyrrolidine-2,4-dione derivative is used for controlling aphids (Aphidina), whiteflies (Tiraleurodes), thrips (Thysanoptera), spider mites (Arachnida), scale insects or mealy-bugs (Coccoidae and Pseudococcoidae).

12. A method according to claim 1, wherein application rates of the 3-arylpyrrolidine-2,4-dione derivative are between 0.1 g/ha and 5.0 kg/ha.

13. A method according to claim 1, wherein the 3-arylpyrrolidine-2,4-dione derivative is present as a mixture with at least one mixing partner.

14. A method according to claim 1, wherein by treating the plant with the 3-arylpyrrolidine-2,4-dione derivative, a kill of harmful organisms of at least 20% in comparison to the control is achieved.

15. Plant parts, in particular seed or propagation material, of transgenic plants, obtainable by a method according to claim 1.

16. Plant parts, in particular seed or propagation material, of transgenic plants, treated by a method according to claim 1.
